(19) United States
(12) Patent Application Publication
(10) Pub. No.: US 2010/0098659 A1
(43) Pub. Date: Apr. 22, 2010

(54) COMPOSITIONS AND METHODS FOR TREATING MATRIX METALLOPROTEINASE 9 (MMP9)-MEDIATED CONDITIONS

(75) Inventors: Richard L. Watson, McPherson, KS (US); Anthony B. Wood, Dallas, TX (US); Gregory J. Archambeau, Puyallap, WA (US)

Correspondence Address: DAVIS WRIGHT TREMAINE, LLP/Seattle, 1201 Third Avenue, Suite 2200, SEATTLE, WA, 98101-3045, US

(73) Assignee: Revalesio Corporation, Tacoma, WA (US)

(21) Appl. No.: 12/604,316

(22) Filed: Oct. 22, 2009

Related U.S. Application Data
(63) Continuation-in-part of application No. 12/258,210, filed on Oct. 24, 2008.
(60) Provisional application No. 61/107,453, filed on Oct. 22, 2008.
(60) Provisional application No. 61/107,480, filed on Oct. 22, 2008.

(51) Int. Cl. A61K 33/14; A61P 25/28; A61P 11/06; A61P 19/02; A61P 35/00; A61P 25/16; A61K 39/395; A61K 38/21

(52) U.S. Cl. 424/85.4; 424/680; 424/130.1

(57) ABSTRACT

Provided are methods for treating an MMP9-mediated condition or disease, comprising administration of an electrokinetically altered aqueous fluid comprising an ionic aqueous solution of charge-stabilized oxygen-containing nanostructures substantially having an average diameter of less than about 100 nanometers and stably configured in the ionic aqueous fluid in an amount sufficient for treating an MMP9-mediated condition or disease. The charge-stabilized oxygen-containing nanostructures are preferably stably configured in the fluid in an amount sufficient to provide for modulation of cellular membrane potential and/or conductivity. Certain aspects comprising modulation or down-regulation of MMP-9 expression and/or activity have utility for treating MMP9-mediated diseases or conditions as disclosed herein (e.g., obstructive airways disease, chronic obstructive pulmonary disease, asthma, rheumatoid arthritis, osteoarthritis, atherosclerosis, cancer, multiple sclerosis, Alzheimer's disease, stroke/cerebral ischemia, head trauma, spinal cord injury, amyotrophic lateral sclerosis, Huntington's disease, Parkinson's disease, migraine, cerebral amyloid angiopathy, AIDS, age-related cognitive decline; mild cognitive impairment and prion diseases).

### CROSS-REFERENCE TO RELATED APPLICATIONS

This application claims the benefit of priority to U.S. Provisional Patent Application Ser. Nos. 61/107,480, and 61/107,453, both filed 22 Oct. 2008, and to U.S. Utility patent application Ser. No. 12/258,210, filed 24 Oct. 2008, both incorporated here in by reference in their entirety.

### FIELD OF THE INVENTION

The present invention relates generally to metalloproteinases and metalloproteinase-mediated conditions, and more particularly to matrix metalloproteinases (MMPs), MMP-mediated conditions, and to MMP inhibitors and treatment of MMP-mediated conditions. Particularly preferred aspects relate to modulation (e.g., inhibition) of MMPs (e.g., MMP9), by administering a therapeutic composition comprising at least one electrokinetically generated fluid (including gas-enriched (e.g., oxygen enriched) electrokinetically generated fluids) as disclosed herein.

### BACKGROUND

Metalloproteinases are a superfamily of proteinases (enzymes) classified into families and subfamilies as described, for example, in N. M. Hooper FEBS Letters 354:1-6, 1994. Examples of metalloproteinases include the matrix metalloproteinases (MMPs) such as the collagenases (MMP1, MMP8, MMP13), the gelatinases (MMP2, MMP9), the stromelysins (MMP3, MMP10, MMP II), matrilysin (MMP7), metalloelastase (MMP12), enamelysin (MMP19), the MT-MMPs (MMP14, MMP15, MMP16, MMP17); the reprolysin or adamalysin or MDC family which includes the secretases and sheddases such as TNF converting enzymes (ADAM10 and TACE); the astacin family which include enzymes such as procollagen processing proteinase (PCP); and other metalloproteinases such as aggrecanase, the endothelin converting enzyme family and the angiotensin converting enzyme family.

Collectively, the metalloproteinases are known to cleave a broad range of matrix substrates such as collagen, proteoglycan and fibronectin. Metalloproteinases are implicated in the processing, or secretion, of biological important cell mediators, such as tumour necrosis factor (TNF); and the post translational proteolysis processing, or shedding, of biologically important membrane proteins, such as the low affinity IgE receptor CD23 (see, e.g., N. M. Hooper et al., Biochem. J. 321:265-279, 1997).

Not surprisingly, therefore, metalloproteinases are believed to be important in many physiological disease processes that involve tissue remodeling (e.g., embryonic development, bone formation, uterine remodelling during menstruation, disrupting the blood-brain barrier etc.). Moreover, inhibition of the activity of one or more metalloproteinases may well be of benefit in these diseases or conditions, for example: various inflammatory and allergic diseases such as, inflammation of the joint (especially rheumatoid arthritis, osteoarthritis and gout), inflammation of the gastro-intestinal tract (especially inflammatory bowel disease, ulcerative colitis and gastritis), inflammation of the skin (especially psoriasis, eczema, dermatitis); in tumour metastasis or invasion; in disease associated with uncontrolled degradation of the extracellular matrix such as osteoarthritis; in bone resorptive disease (such as osteoporosis and Paget's disease); in diseases associated with aberrant angiogenesis; the enhanced collagen remodelling associated with diabetes, periodontal disease (such as gingivitis), corneal ulceration, ulceration of the skin, post-operative conditions (such as colonic anastomosis) and dermal wound healing; demyelinating diseases of the central and peripheral nervous systems (such as multiple sclerosis); Alzieimer's disease; extracellular matrix remodelling observed in cardiovascular diseases such as restenosis and atherosclerosis; asthma; rhinitis; and chronic obstructive pulmonary diseases (COPD).

MMP12, also known as macrophage elastase or metalloelastase, was initially cloned in the mouse (Shapiro et al., Journal of Biological Chemistry 267: 4664, 1992) and has also been cloned in man by the same group in 1995. MMP12 is preferentially expressed in activated macrophages, and has been shown to be secreted from alveolar macrophages from smokers (Shapiro et al, 1993, Journal of Biological Chemistry, 268: 23824) as well as in foam cells in atherosclerotic lesions (Matsumoto et al, Am. J. Pathol. 153: 109, 1998). A mouse model of COPD is based on challenge of mice with cigarette smoke for six months, two cigarettes a day six days a week. Wild-type mice developed pulmonary emphysema after this treatment. When MMP12 knock-out mice were tested in this model they developed no significant emphysema, strongly indicating that MMP12 is a key enzyme in the COPD pathogenesis. The role of MMPs such as MMP12 in COPD (emphysema and bronchitis) is discussed in Anderson and Shinagawa, 1999, Current Opinion in Anti-inflammatory and Immunomodulatory Investigational Drugs 1(1): 29-38. It was recently discovered that smoking increases macrophage infiltration and macrophage-derived MMP-12 expression in human carotid artery plaques (Matetzky S, Fishbein M C et al., Circulation 102:(18), 36-39 Suppl. S, Oct. 31, 2000).

MMP9-(Gelatinase B; 92 kDa-TypeIV Collagenase; 92 kDa Gelatinase) is a secreted protein which was first purified, then cloned and sequenced, in 1989 (S. M. Wilhelm et al., J. Biol. Chem. 264 (29): 17213-17221, 1989; published erratum in J. Biol. Chem. 265 (36): 22570, 1990) (for review of detailed information and references on this protease see T. H. Vu & Z. Werb (1998) (In: Matrix Metalloproteinases, 1998, edited by W. C. Parks & R. P. Mecham, pp. 115-148, Academic Press. ISBN 0-12-545090-7).

The expression of MMP9 is restricted normally to a few cell types, including trophoblasts, osteoclasts, neutrophils and macrophages (Vu & Werb, supra). However, the expression can be induced in these same cells and in other cell types by several mediators, including exposure of the cells to growth factors or cytokines. These are the same mediators often implicated in initiating an inflammatory response. As with other secreted MMPs, MMP9 is released as an inactive Pro-enzyme, which is subsequently cleaved to form the enzymatically active enzyme. The proteases required for this activation in vivo are not known. The balance of active MMP9 versus inactive enzyme is further regulated in vivo by interaction with TIMP-1 (Tissue Inhibitor of Metalloproteinases-1), a naturally-occurring protein. TIMP-1 binds to the C-terminal region of MMP9, leading to inhibition of the catalytic domain of MMP9. The balance of induced expression of ProMMP9, cleavage of Pro- to active MMP9 and the presence of TIMP-1 combine to determine the amount of catalytically active MMP9 which is present at a local site. Proteolytically active MMP9 attacks substrates which include gelatin, elastin, and native Type IV and Type V collagens; it has no activity against native Type I collagen, proteoglycans or laminins.

There has been a growing body of data implicating roles for MMP9 in various physiological and pathological processes. Physiological roles include the invasion of embryonic trophoblasts through the uterine epithelium in the early stages of embryonic implantation; some role in the growth and development of bones; and migration of inflammatory cells from the vasculature into tissues.

MMP9 release, measured using enzyme immunoassay, was significantly enhanced in fluids and in AM supernantants from untreated asthmatics compared with those from other populations (Am. J. Resp. Cell & Mol. Biol., 5:583-591, 1997). Also, increased MMP9 expression has been observed in certain other pathological conditions, thereby implicating MMP9 in disease processes such as COPD, arthritis, tumour metastasis, Alzheimer's disease, multiple sclerosis, and plaque rupture in atherosclerosis leading to acute coronary conditions such as myocardial infarction (see also WO07087637A3, incorporated herein by reference).

Recently, it has been demonstrated that the levels of MMP-9 are significantly increased in patients with stable asthma and even higher in patients with acute asthmatic patients compared with healthy control subjects. MMP-9 plays a crucial role in the infiltration of airway inflammatory cells and the induction of airway hyperresponsiveness indicating that MMP-9 may have an important role in inducing and maintaining asthma (Vignola et al., Sputum metalloproteinase-9/tissue inhibitor of metalloproteinase-1 ratio correlates with airflow obstruction in asthma and chronic bronchitis, Am J Respir Crit Care Med 158:1945-1950, 1998; Hoshino et al., Inhaled corticosteroids decrease subepithelial collagen deposition by modulation of the balance between matrix metalloproteinase-9 and tissue inhibitor of metalloproteinase-1 expression in asthma, J Allergy Clin Immunol 104:356-363, 1999; Simpson et al., Differential proteolytic enzyme activity in eosinophilic and neutrophilic asthma, Am J Respir Crit Care Med 172:559-565, 2005; Lee et al., A murine model of toluene diisocyanate-induced asthma can be treated with matrix metalloproteinase inhibitor, J Allergy Clin Immunol 108:1021-1026, 2001; and Lee et al., Matrix metalloproteinase inhibitor regulates inflammatory cell migration by reducing ICAM-1 and VCAM-1 expression in a murine model of toluene diisocyanate-induced asthma, J Allergy Clin Immunol 2003; 111:1278-1284).

### SUMMARY OF EXEMPLARY EMBODIMENTS

Particular aspects provide a method for treating an MMP9-mediated condition or disease, comprising administration to a mammal in need thereof, a therapeutically effective amount of an electrokinetically altered aqueous fluid comprising an ionic aqueous solution of charge-stabilized oxygen-containing nanostructures substantially having an average diameter of less than about 100 nanometers and stably configured in the ionic aqueous fluid in an amount sufficient for treating an MMP9-mediated condition or disease. In certain aspects, the charge-stabilized oxygen-containing nanostructures are stably configured in the ionic aqueous fluid in an amount sufficient to provide, upon contact of a living cell by the fluid, modulation of at least one of cellular membrane potential and cellular membrane conductivity.

In certain method aspects, the charge-stabilized oxygen-containing nanostructures are the major charge-stabilized gas-containing nanostructure species in the fluid. In particular embodiments, the percentage of dissolved oxygen molecules present in the fluid as the charge-stabilized oxygen-containing nanostructures is a percentage selected from the group consisting of greater than: 0.01%, 0.1%, 1%, 5%; 10%; 15%; 20%; 25%; 30%; 35%; 40%; 45%; 50%; 55%; 60%; 65%; 70%; 75%; 80%; 85%; 90%; and 95%. In particular aspects, the total dissolved oxygen is substantially present in the charge-stabilized oxygen-containing nanostructures. In certain embodiments, the charge-stabilized oxygen-containing nanostructures substantially have an average diameter of less than a size selected from the group consisting of: 90 nm; 80 nm; 70 nm; 60 nm; 50 nm; 40 nm; 30 nm; 20 nm; 10 nm; and less than 5 nm.

In particular method aspects, the ionic aqueous solution comprises a saline solution. In certain aspects, the electrokinetically-altered aqueous fluid is superoxygenated.

In particular method aspects, the electrokinetically-altered aqueous fluid comprises a form of solvated electrons.

In certain aspects, alteration of the electrokinetically-altered aqueous fluid comprises exposure of the fluid to hydrodynamically-induced, localized electrokinetic effects. In certain embodiments, exposure to the localized electrokinetic effects comprises exposure to at least one of voltage pulses and current pulses. In particular aspects, exposure of the fluid to hydrodynamically-induced, localized electrokinetic effects, comprises exposure of the fluid to electrokinetic effect-inducing structural features of a device used to generate the fluid.

In particular aspects, the MMP9-mediated condition or disease comprises an obstructive airways disease, including but not limited to asthma and chronic obstructive pulmonary disease. In particular aspects, the MMP9-mediated condition or disease comprises at least one of rheumatoid arthritis, osteoarthritis, atherosclerosis, cancer, and multiple sclerosis. In certain embodiments, the MMP9-mediated condition or disease comprises at least one disease or disorder of the peripheral or central nervous system characterized by persistent or sustained expression and/or activity of MMP9, selected from the group consisting of Alzheimer's disease, stroke/cerebral ischemia, head trauma, spinal cord injury, multiple sclerosis, amyotrophic lateral sclerosis, Huntington's disease, Parkinson's disease, migraine, cerebral amyloid angiopathy, AIDS, age-related cognitive decline; mild cognitive impairment and prion diseases in a mammal.

In certain aspects, the method further comprises combination therapy, wherein at least one additional therapeutic agent is administered to the patient. In particular embodiments, the at least one additional therapeutic agent comprises administration of an additional inhibitor of at least one MMP, In certain aspects, the at least one MMP is selected from the group consisting of MMP-1, MMP-2, MMP-7, MMP-8, MMP-9, MMP-10, MMP-11, MMP-12, MMP-13, MMP-14, MMP-15, MMP-16, MMP-17, MMP-18, MMP-19 and MMP-20 MMP-1, MMP-2, MMP-3, MMP-7, MMP-8, MMP-9, MMP-10, MMP-11, MMP-12, MMP-13, MMP-14, MMP-15, MMP-16, MMP-17, MMP-18, MMP-19 and MMP-20. In particular aspects, the at least one additional therapeutic agent is a TSLP and/or TSLPR antagonist, and in particular embodiments, the TSLP and/or TSLPR antagonist is selected from the group consisting of neutralizing antibodies specific for TSLP and the TSLP receptor, soluble TSLP receptor molecules, and TSLP receptor fusion proteins, including TSLPR-immunoglobulin Fc molecules or polypeptides that encode components of more than one receptor chain.

In certain aspects, the at least one additional therapeutic agent is selected from the group consisting of: standard non-steroidal anti-inflammatory drugs (NSAID'S), piroxicam, diclofenac; a propionic acid, naproxen, flubiprofen, fenoprofen, ketoprofen and ibuprofen; a fenamate, mefenamic acid, indomethacin, sulindac, apazone; a pyrazolone, phenylbutazone; a salicylate, aspirin; an analgesic or intra-articular therapy, a corticosteroid; a hyaluronic acid, hyalgan, synvisc; an immune suppressant, cyclosporine, interferon; a TNF-.alpha inhibitor, Enbrel™; low dose methotrexate, lefunimide, hydroxychloroquine, d-penicilamine, auranofin, parenteral gold and oral gold.

In particular embodiments, the at least one additional therapeutic agent is selected from the CNS agent group consisting of: an antidepressant, sertraline, fluoxetine, paroxetine; an anti-Parkinsonian drug; deprenyl, L-dopa, requip, miratex; a MAOB inhibitor, selegine, rasagiline; a COMP inhibitor, tolcapone, Tasmar; an A-2 inhibitor, a dopamine reuptake inhibitor, an NMDA antagonist, a nicotine agonist, a dopamine agonist, an inhibitor of neuronal nitric oxide synthase, an anti-Alzheimer's drug; an acetylcholinesterase inhibitor, metrifonate, donepezil, Aricept, Exelon, ENA 713 or rivastigmine; tetrahydroaminoacridine, Tacrine, Cognex, or THA; a COX-1 or COX-2 inhibitor, celecoxib, Celebrex, rofecoxib, Vioxx; propentofylline, an anti-stroke medication, an NR2B selective antagonist, a glycine site antagonist, and a neutrophil inhibitory factor (NIF).

In certain aspects, the at least one additional therapeutic agent is selected from the group consisting of: an estrogen; a selective estrogen modulator, estrogen, raloxifene, tamoxifene, droloxifene, lasofoxifene; an agent that results in reduction of A.beta.1-40/1-42, an amyloid aggregation inhibitor, a secretase inhibitor; an osteoporosis agent, droloxifene, fosomax; immunosuppressant agents, FK-506, rapamycin; an anticancer agent, endostatin, angiostatin; a cytotoxic drug, adriamycin, daunomycin, cis-platinum, etoposide, taxol, taxotere; an alkaloid, vincristine; an antimetabolite, methotrexate; a cardiovascular agent, calcium channel blockers; a lipid lowering agent, a statin; a fibrate, a beta-blocker, an ACE inhibitor, an angiotensin-2 receptor antagonist, and a platelet aggregation inhibitor.

In particular method aspects, modulation of at least one of cellular membrane potential and cellular membrane conductivity comprises altering at least one of cellular membrane structure or function comprising altering at least one of a conformation, ligand binding activity, and a catalytic activity of a membrane associated protein or constituent. In certain aspects, the membrane associated protein comprises at least one selected from the group consisting of receptors, transmembrane receptors, ion channel proteins, intracellular attachment proteins, cellular adhesion proteins, integrins, etc. In certain embodiments, the transmembrane receptor comprises a G-Protein Coupled Receptor (GPCR). In particular aspects, the G-Protein Coupled Receptor (GPCR) interacts with a G protein α subunit, for example, wherein the G protein α subunit comprises at least one selected from the group consisting of Gαₛ, Gαᵢ, Gα_{q}, and Gα₁₂, and in certain embodiments the at least one G protein α subunit is Gα_{q}.

In particular method aspects, modulation of at least one of cellular membrane potential and cellular membrane conductivity comprises modulating whole-cell conductance, for example, wherein modulating whole-cell conductance comprises modulating at least one of a linear and a non-linear voltage-dependent contribution of the whole-cell conductance.

In certain method aspects, modulation of at least one of cellular membrane potential and cellular membrane conductivity comprises modulation of a calcium dependant cellular messaging pathway or system. In certain method aspects, modulation of at least one of cellular membrane potential and cellular membrane conductivity comprises modulation of phospholipase C activity. In certain method aspects, modulation of at least one of cellular membrane potential and cellular membrane conductivity comprises modulation of adenylate cyclase (AC) activity. In certain method aspects, modulation of at least one of cellular membrane potential and cellular membrane conductivity comprises modulation of intracellular signal transduction associated with at least one condition or symptom selected from the group consisting of obstructive airways disease, chronic obstructive pulmonary disease, asthma, rheumatoid arthritis, osteoarthritis, atherosclerosis, cancer, multiple sclerosis, Alzheimer's disease, stroke/cerebral ischemia, head trauma, spinal cord injury, amyotrophic lateral sclerosis, Huntington's disease, Parkinson's disease, migraine, cerebral amyloid angiopathy, AIDS, age-related cognitive decline; mild cognitive impairment and prion diseases.

Particular method aspects comprise administration of the electrokinetic fluid to a cell network or layer, and further comprise modulation of an intercellular junction therein. In certain embodiments, the intracellular junction comprises at least one selected from the group consisting of tight junctions, gap junctions, zona adherins and desmasomes. In particular aspects, the cell network or layers comprise at least one selected from the group consisting of pulmonary epithelium, bronchial epithelium, and intestinal epithelium.

In certain method aspects, the electrokinetically altered aqueous fluid is oxygenated, wherein the oxygen in the fluid is present in an amount of at least 8 ppm, at least 15, ppm, at least 25 ppm, at least 30 ppm, at least 40 ppm, at least 50 ppm, or at least 60 ppm oxygen at atmospheric pressure.

In certain method aspects, the electrokinetically altered aqueous fluid comprises at least one of solvated electrons, and electrokinetically modified or charged oxygen species, for example, wherein the form of solvated electrons or electrokinetically modified or charged oxygen species are present in an amount of at least 0.01 ppm, at least 0.1 ppm, at least 0.5 ppm, at least 1 ppm, at least 3 ppm, at least 5 ppm, at least 7 ppm, at least 10 ppm, at least 15 ppm, or at least 20 ppm. In certain aspects, the electrokinetically altered aqueous fluid comprises a form of solvated electrons stabilized by molecular oxygen.

In certain aspects, the ability of the electrokinetically-altered fluid to modulate at least one of cellular membrane potential and cellular membrane conductivity persists for at least two, at least three, at least four, at least five, at least 6, at least 12 months, or longer periods, in a closed gas-tight container.

In certain aspects, the amount of oxygen present in charge-stabilized oxygen-containing nanostructures of the electrokinetically-altered fluid is at least 8 ppm, at least 15, ppm, at least 20 ppm, at least 25 ppm, at least 30 ppm, at least 40 ppm, at least 50 ppm, or at least 60 ppm oxygen at atmospheric pressure.

In particular aspects, treating comprises administration by at least one of topical, inhalation, intranasal, and intravenous.

### BRIEF DESCRIPTION OF THE DRAWINGS

FIG. 1 shows that the inventive electrokinetically generated fluid (e.g., Revera 60 and Solas) reduced DEP-induced TSLP receptor expression in bronchial epithelial cells (BEC) by approximately 90% and 50%, respectively, whereas normal saline (NS) had only a marginal effect.

FIG. 2 shows the inventive electrokinetically generated fluid (e.g., Revera 60 and Solas) inhibited the DEP-induced cell surface bound MMP9 levels in bronchial epithelial cells by approximately 80%, and 70%, respectively, whereas normal saline (NS) had only a marginal effect.

FIGS. 3 A-C demonstrate the results of a series of patch clamping experiments that assessed the effects of the electrokinetically generated fluid (e.g., RNS-60 and Solas) on epithelial cell membrane polarity and ion channel activity at two time-points (15 min (left panels) and 2 hours (right panels)) and at different voltage protocols.

FIGS. 4 A-C show, in relation to the experiments relating to FIGS. 3 A-C, the graphs resulting from the subtraction of the Solas current data from the RNS-60 current data at three voltage protocols (A. stepping from zero mV; B. stepping from −60 mV; C stepping from −120 mV) and the two time-points (15 minutes (open circles) and 2 hours (closed circles)).

FIGS. 5 A-D demonstrate the results of a series of patch clamping experiments that assessed the effects of the electrokinetically generated fluid (e.g., Solas (panels A and B) and RNS-60 (panels C. and D.)) on epithelial cell membrane polarity and ion channel activity using different external salt solutions and at different voltage protocols (panels A and C show stepping from zero mV; panels B and D show stepping from −120 mV).

FIGS. 6 A-D show, in relation to the experiments relating to FIGS. 5 A-D, the graphs resulting from the subtraction of the CsCl current data (shown in FIG. 5) from the 20 mM CaCl₂ (diamonds) and 40 mM CaCl₂ (filled squares) current data at two voltage protocols (panels A and C stepping from zero mV; B and D stepping from −120 mV) for Solas (panels A and B) and Revera 60 (panels C and D).

FIGS. 7A and B demonstrate the results of a patch clamp experiment that assessed the effects of diluting the electrokinetically generated fluid (e.g., RNS-60) on epithelial cell membrane polarity and ion channel activity.

### DETAILED DESCRIPTION

Provided are methods for treating an MMP9-mediated condition or disease, comprising administration of an electrokinetically altered aqueous fluid comprising an ionic aqueous solution of charge-stabilized oxygen-containing nanostructures substantially having an average diameter of less than about 100 nanometers and stably configured in the ionic aqueous fluid in an amount sufficient for treating an MMP9-mediated condition or disease. The charge-stabilized oxygen-containing nanostructures are preferably stably configured in the fluid in an amount sufficient to provide for modulation of cellular membrane potential and/or conductivity. Certain aspects comprising modulation or clown-regulation of MMP-9 expression and/or activity have utility for treating MMP9-mediated diseases or conditions as disclosed herein (e.g., obstructive airways disease, chronic obstructive pulmonary disease, asthma, rheumatoid arthritis, osteoarthritis, atherosclerosis, cancer, multiple sclerosis, Alzheimer's disease, stroke/cerebral ischemia, head trauma, spinal cord injury, amyotrophic lateral sclerosis, Huntington's disease, Parkinson's disease, migraine, cerebral amyloid angiopathy, AIDS, age-related cognitive decline; mild cognitive impairment and prion diseases).

Methods for making the electrokinetically generated fluid used herein have been previously described (see, e.g., US-2008-0219088, PCT/US2007/082578), which are incorporated herein by reference in their entirety).

Particular aspects provide methods for treating an MMP (e.g., MMP9)-mediated disease or condition, comprising administering, to a subject in need thereof, a therapeutically effective amount of a composition comprising at least one electrokinetically generated fluid (including gas-enriched (e.g., oxygen enriched) electrokinetically generated fluids) as disclosed herein.

Additional aspects provide methods for treating an obstructive airways disease, comprising administering a therapeutic composition comprising at least one electrokinetically generated fluid (including gas-enriched (e.g., oxygen enriched) electrokinetically generated fluids) as disclosed herein. In particular embodiments, the obstructive airways disease comprises at least one of asthma and chronic obstructive pulmonary disease. Particular aspects comprise treating at least one of rheumatoid arthritis, osteoarthritis, atherosclerosis, cancer, and multiple sclerosis.

Additional aspects provide novel methods and compositions having substantial utility for treatment of COPD, arthritis, tumor metastasis, Alzheimer's disease, multiple sclerosis, and plaque rupture in atherosclerosis leading to acute coronary conditions such as myocardial infarction. The novel methods have utility for treating a disease or disorder of the peripheral or central nervous system characterized by persistent or sustained expression and/or activity of MMP9, including but not limited to Alzheimer's disease, stroke/cerebral ischemia, head trauma, spinal cord injury, multiple sclerosis, amyotrophic lateral sclerosis, Huntington's disease, Parkinson's disease, migraine, cerebral amyloid angiopathy, AIDS, age-related cognitive decline; mild cognitive impairment and prion diseases in a mammal, which comprises administering to said mammal a therapeutically effective amount of a MMP9 inhibitor as described herein.

Particular aspects of the present invention provide novel methods and compositions having substantial utility for treatment of cognitive impairment (e.g., dementia, cognitive decline in aged individuals, Alzheimer's disease, etc.). Preferably, inhibitors of MMP-9 as described herein are used.

Additional aspects provide methods treating an MMP9-mediated disease or condition, comprising administering, to a subject in need thereof, a therapeutically effective amount of a composition comprising at least one electrokinetically generated fluid (including gas-enriched (e.g., oxygen enriched) electrokinetically generated fluids) as disclosed herein, in combination with administration of an additional inhibitor of at least one other MMP (e.g., an inhibitor of MMP-1, MMP-2, MMP-7, MMP-8, MMP-9, MMP-10, MMP-11, MMP-12, MMP-13, MMP-14, MMP-15, MMP-16, MMP-17, MMP-18, MMP-19 and MMP-20 MMP-1, MMP-2, MMP-3, MMP-7, MMP-8, MMP-9, MMP-10, MMP-11, MMP-12, MMP-13, MMP-14, MMP-15, MMP-16, MMP-17, MMP-18, MMP-19 and MMP-20, etc.). In particular embodiments, the at least one additional matrix metalloproteinase (MMP) inhibitor is suitable to inhibit at least one MMP having the characteristic elevated expression or activity. In additional embodiments, the at least one matrix metalloproteinase (MMP) inhibitor is suitable to inhibit at least two MMPs having the characteristic elevated expression or activity. In certain embodiments, the at least one matrix metalloproteinase (MMP) inhibitor is MMP-specific or substantially specific to a particular MMP or inhibits a limited number of MMPs (e.g., from one to two MMPs, from one to three MMPs, or from about one to about four MMPs). In certain embodiments the at least one matrix metalloproteinase (MMP) inhibitor is a broad spectrum MMP inhibitor inhibiting at least three, or at least 4 MMPs (e.g., having the characteristic elevated expression or activity).

### Preferred Exemplary Embodiments

Particular aspects provide a method for treating an MMP9-mediated condition or disease, comprising administration to a mammal in need thereof, a therapeutically effective amount of an electrokinetically altered aqueous fluid comprising an ionic aqueous solution of charge-stabilized oxygen-containing nanostructures substantially having an average diameter of less than about 100 nanometers and stably configured in the ionic aqueous fluid in an amount sufficient for treating an MMP9-mediated condition or disease. In certain aspects, the charge-stabilized oxygen-containing nanostructures are stably configured in the ionic aqueous fluid in an amount sufficient to provide, upon contact of a living cell by the fluid, modulation of at least one of cellular membrane potential and cellular membrane conductivity.

In certain method aspects, the charge-stabilized oxygen-containing nanostructures are the major charge-stabilized gas-containing nanostructure species in the fluid. In particular embodiments, the percentage of dissolved oxygen molecules present in the fluid as the charge-stabilized oxygen-containing nanostructures is a percentage selected from the group consisting of greater than: 0.01%, 0.1%, 1%, 5%; 10%; 15%; 20%; 25%; 30%; 35%; 40%; 45%; 50%; 55%; 60%; 65%; 70%; 75%; 80%; 85%; 90%; and 95%. In particular aspects, the total dissolved oxygen is substantially present in the charge-stabilized oxygen-containing nanostructures. In certain embodiments, the charge-stabilized oxygen-containing nanostructures substantially have an average diameter of less than a size selected from the group consisting of: 90 nm; 80 nm; 70 nm; 60 nm; 50 nm; 40 nm; 30 nm; 20 nm; 10 nm; and less than 5 nm.

In particular method aspects, the ionic aqueous solution comprises a saline solution. In certain aspects, the electrokinetically-altered aqueous fluid is superoxygenated.

In particular method aspects, the electrokinetically-altered aqueous fluid comprises a form of solvated electrons.

In certain aspects, alteration of the electrokinetically-altered aqueous fluid comprises exposure of the fluid to hydrodynamically-induced, localized electrokinetic effects. In certain embodiments, exposure to the localized electrokinetic effects comprises exposure to at least one of voltage pulses and current pulses. In particular aspects, exposure of the fluid to hydrodynamically-induced, localized electrokinetic effects, comprises exposure of the fluid to electrokinetic effect-inducing structural features of a device used to generate the fluid.

In particular aspects, the MMP9-mediated condition or disease comprises an obstructive airways disease, including but not limited to asthma and chronic obstructive pulmonary disease. In particular aspects, the MMP9-mediated condition or disease comprises at least one of rheumatoid arthritis, osteoarthritis, atherosclerosis, cancer, and multiple sclerosis. In certain embodiments, the MMP9-mediated condition or disease comprises at least one disease or disorder of the peripheral or central nervous system characterized by persistent or sustained expression and/or activity of MMP9, selected from the group consisting of Alzheimer's disease, stroke/cerebral ischemia, head trauma, spinal cord injury, multiple sclerosis, amyotrophic lateral sclerosis, Huntington's disease, Parkinson's disease, migraine, cerebral amyloid angiopathy, AIDS, age-related cognitive decline; mild cognitive impairment and prion diseases in a mammal.

In certain aspects, the method further comprises combination therapy, wherein at least one additional therapeutic agent is administered to the patient. In particular embodiments, the at least one additional therapeutic agent comprises administration of an additional inhibitor of at least one MMP. In certain aspects, the at least one MMP is selected from the group consisting of MMP-1, MMP-2, MMP-7, MMP-8, MMP-9, MMP-10, MMP-11, MMP-12, MMP-13, MMP-14, MMP-15, MMP-16, MMP-17, MMP-18, MMP-19 and MMP-20 MMP-1, MMP-2, MMP-3, MMP-7, MMP-8, MMP-9, MMP-10, MMP-11, MMP-12, MMP-13, MMP-14, MMP-15, MMP-16, MMP-17, MMP-18, MMP-19 and MMP-20. In particular aspects, the at least one additional therapeutic agent is a TSLP and/or TSLPR antagonist, and in particular embodiments, the TSLP and/or TSLPR antagonist is selected from the group consisting of neutralizing antibodies specific for TSLP and the TSLP receptor, soluble TSLP receptor molecules, and TSLP receptor fusion proteins, including TSLPR-immunoglobulin Fc molecules or polypeptides that encode components of more than one receptor chain.

In certain aspects, the at least one additional therapeutic agent is selected from the group consisting of: standard non-steroidal anti-inflammatory drugs (NSAID'S), piroxicam, diclofenac; a propionic acid, naproxen, flubiprofen, fenoprofen, ketoprofen and ibuprofen; a fenamate, mefenamic acid, indomethacin, sulindac, apazone; a pyrazolone, phenylbutazone; a salicylate, aspirin; an analgesic or intra-articular therapy, a corticosteroid; a hyaluronic acid, hyalgan, synvisc; an immune suppressant, cyclosporine, interferon; a TNF-.alpha inhibitor, Enbrel™; low dose methotrexate, lefunimide, hydroxychloroquine, d-penicilamine, auranofin, parenteral gold and oral gold.

In particular embodiments, the at least one additional therapeutic agent is selected from the CNS agent group consisting of: an antidepressant, sertraline, fluoxetine, paroxetine; an anti-Parkinsonian drug; deprenyl, L-dopa, requip, miratex; a MAOB inhibitor, selegine, rasagiline; a COMP inhibitor, tolcapone, Tasmar; an A-2 inhibitor, a dopamine reuptake inhibitor, an NMDA antagonist, a nicotine agonist, a dopamine agonist, an inhibitor of neuronal nitric oxide synthase, an anti-Alzheimer's drug; an acetylcholinesterase inhibitor, metrifonate, donepezil, Aricept, Exelon, ENA 713 or rivastigmine; tetrahydroaminoacridine, Tacrine, Cognex, or THA; a COX-1 or COX-2 inhibitor, celecoxib, Celebrex, rofecoxib, Vioxx; propentofylline, an anti-stroke medication, an NR2B selective antagonist, a glycine site antagonist, and a neutrophil inhibitory factor (NIF).

In certain aspects, the at least one additional therapeutic agent is selected from the group consisting of: an estrogen; a selective estrogen modulator, estrogen, raloxifene, tamoxifene, droloxifene, lasofoxifene; an agent that results in reduction of A.beta.1-40/1-42, an amyloid aggregation inhibitor, a secretase inhibitor; an osteoporosis agent, droloxifene, fosomax; immunosuppressant agents, FK-506, rapamycin; an anticancer agent, endostatin, angiostatin; a cytotoxic drug, adriamycin, daunomycin, cis-platinum, etoposide, taxol, taxotere; an alkaloid, vincristine; an antimetabolite, methotrexate; a cardiovascular agent, calcium channel blockers; a lipid lowering agent, a statin; a fibrate, a beta-blocker, an ACE inhibitor, an angiotensin-2 receptor antagonist, and a platelet aggregation inhibitor.

In particular method aspects, modulation of at least one of cellular membrane potential and cellular membrane conductivity comprises altering at least one of cellular membrane structure or function comprising altering at least one of a conformation, ligand binding activity, and a catalytic activity of a membrane associated protein or constituent. In certain aspects, the membrane associated protein comprises at least one selected from the group consisting of receptors, transmembrane receptors, ion channel proteins, intracellular attachment proteins, cellular adhesion proteins, integrins, etc. In certain embodiments, the transmembrane receptor comprises a G-Protein Coupled Receptor (GPCR). In particular aspects, the G-Protein Coupled Receptor (GPCR) interacts with a G protein α subunit, for example, wherein the G protein α subunit comprises at least one selected from the group consisting of Gαₛ, Gαᵢ, Gα_{q}, and Gα₁₂, and in certain embodiments the at least one G protein α subunit is Gα_{q}.

In particular method aspects, modulation of at least one of cellular membrane potential and cellular membrane conductivity comprises modulating whole-cell conductance, for example, wherein modulating whole-cell conductance comprises modulating at least one of a linear and a non-linear voltage-dependent contribution of the whole-cell conductance.

In certain method aspects, modulation of at least one of cellular membrane potential and cellular membrane conductivity comprises modulation of a calcium dependant cellular messaging pathway or system. In certain method aspects, modulation of at least one of cellular membrane potential and cellular membrane conductivity comprises modulation of phospholipase C activity. In certain method aspects, modulation of at least one of cellular membrane potential and cellular membrane conductivity comprises modulation of adenylate cyclase (AC) activity. In certain method aspects, modulation of at least one of cellular membrane potential and cellular membrane conductivity comprises modulation of intracellular signal transduction associated with at least one condition or symptom selected from the group consisting of obstructive airways disease, chronic obstructive pulmonary disease, asthma, rheumatoid arthritis, osteoarthritis, atherosclerosis, cancer, multiple sclerosis, Alzheimer's disease, stroke/cerebral ischemia, head trauma, spinal cord injury, amyotrophic lateral sclerosis, Huntington's disease, Parkinson's disease, migraine, cerebral amyloid angiopathy, AIDS, age-related cognitive decline; mild cognitive impairment and prion diseases.

Particular method aspects comprise administration of the electrokinetic fluid to a cell network or layer, and further comprise modulation of an intercellular junction therein. In certain embodiments, the intracellular junction comprises at least one selected from the group consisting of tight junctions, gap junctions, zona adherins and desmasomes. In particular aspects, the cell network or layers comprise at least one selected from the group consisting of pulmonary epithelium, bronchial epithelium, and intestinal epithelium.

In certain method aspects, the electrokinetically altered aqueous fluid is oxygenated, wherein the oxygen in the fluid is present in an amount of at least 8 ppm, at least 15, ppm, at least 25 ppm, at least 30 ppm, at least 40 ppm, at least 50 ppm, or at least 60 ppm oxygen at atmospheric pressure.

In certain method aspects, the electrokinetically altered aqueous fluid comprises at least one of solvated electrons, and electrokinetically modified or charged oxygen species, for example, wherein the form of solvated electrons or electrokinetically modified or charged oxygen species are present in an amount of at least 0.01 ppm, at least 0.1 ppm, at least 0.5 ppm, at least 1 ppm, at least 3 ppm, at least 5 ppm, at least 7 ppm, at least 10 ppm, at least 15 ppm, or at least 20 ppm. In certain aspects, the electrokinetically altered aqueous fluid comprises a form of solvated electrons stabilized by molecular oxygen.

In certain aspects, the ability of the electrokinetically-altered fluid to modulate at least one of cellular membrane potential and cellular membrane conductivity persists for at least two, at least three, at least four, at least five, at least 6, at least 12 months, or longer periods, in a closed gas-tight container.

In certain aspects, the amount of oxygen present in charge-stabilized oxygen-containing nanostructures of the electrokinetically-altered fluid is at least 8 ppm, at least 15, ppm, at least 20 ppm, at least 25 ppm, at least 30 ppm, at least 40 ppm, at least 50 ppm, or at least 60 ppm oxygen at atmospheric pressure.

In particular aspects, treating comprises administration by at least one of topical, inhalation, intranasal, and intravenous.

### Electrokinetically-Generated Fluids:

“Electrokinetically generated fluid,” as used herein, refers to Applicants' inventive electrokinetically-generated fluids generated, for purposes of the working Examples herein, by the exemplary Mixing Device described in detail herein (see also US200802190088 and WO2008/052143, both incorporated herein by reference in their entirety). The electrokinetic fluids, as demonstrated by the data disclosed and presented herein, represent novel and fundamentally distinct fluids relative to prior art non-electrokinetic fluids, including relative to prior art oxygenated non-electrokinetic fluids (e.g., pressure pot oxygenated fluids and the like). As disclosed in various aspects herein, the electrokinetically-generated fluids have unique and novel physical and biological properties including, but not limited to the following:

In particular aspects, the electrokinetically altered aqueous fluid comprise an ionic aqueous solution of charge-stabilized oxygen-containing nanostructures substantially having an average diameter of less than about 100 nanometers and stably configured in the ionic aqueous fluid in an amount sufficient to provide, upon contact of a living cell by the fluid, modulation of at least one of cellular membrane potential and cellular membrane conductivity.

In particular aspects, electrokinetically-generated fluids refers to fluids generated in the presence of hydrodynamically-induced, localized (e.g., non-uniform with respect to the overall fluid volume) electrokinetic effects (e.g., voltage/current pulses), such as device feature-localized effects as described herein. In particular aspects said hydrodynamically-induced, localized electrokinetic effects are in combination with surface-related double layer and/or streaming current effects as disclosed and discussed herein.

In particular aspects, the electrokinetically altered aqueous fluids are suitable to modulate ¹³C-NMR line-widths of reporter solutes (e.g., trehelose) dissolved therein. NMR line-width effects are in indirect method of measuring, for example, solute ‘tumbling’ in a test fluid as described herein in particular working Examples.

In particular aspects, the electrokinetically altered aqueous fluids are characterized by at least one of: distinctive square wave voltametry peak differences at any one of −0.14V, −0.47V, −1.02V and −1.36V; polarographic peaks at −0.9 volts; and an absence of polarographic peaks at −0.19 and −0.3 volts, which are unique to the electrokinetically generated fluids as disclosed herein in particular working Examples.

In particular aspects, the electrokinetically altered aqueous fluids are suitable to alter cellular membrane conductivity (e.g., a voltage-dependent contribution of the whole-cell conductance as measure in patch clamp studies disclosed herein).

In particular aspects, the electrokinetically altered aqueous fluids are oxygenated, wherein the oxygen in the fluid is present in an amount of at least 15, ppm, at least 25 ppm, at least 30 ppm, at least 40 ppm, at least 50 ppm, or at least 60 ppm dissolved oxygen at atmospheric pressure. In particular aspects, the electrokinetically altered aqueous fluids have less than 15 ppm, less that 10 ppm of dissolved oxygen at atmospheric pressure, or approximately ambient oxygen levels.

In particular aspects, the electrokinetically altered aqueous fluids are oxygenated, wherein the oxygen in the fluid is present in an amount between approximately 8 ppm and approximately 15 ppm, and in this case is sometimes referred to herein as “Solas.”

In particular aspects, the electrokinetically altered aqueous fluid comprises at least one of solvated electrons (e.g., stabilized by molecular oxygen), and electrokinetically modified and/or charged oxygen species, and wherein in certain embodiments the solvated electrons and/or electrokinetically modified or charged oxygen species are present in an amount of at least 0.01 ppm, at least 0.1 ppm, at least 0.5 ppm, at least 1 ppm, at least 3 ppm, at least 5 ppm, at least 7 ppm, at least 10 ppm, at least 15 ppm, or at least 20 ppm.

In particular aspects, the electrokinetically altered aqueous fluids are suitable to alter cellular membrane structure or function (e.g., altering of a conformation, ligand binding activity, or a catalytic activity of a membrane associated protein) sufficient to provide for modulation of intracellular signal transduction, wherein in particular aspects, the membrane associated protein comprises at least one selected from the group consisting of receptors, transmembrane receptors (e.g., G-Protein Coupled Receptor (GPCR), TSLP receptor, beta 2 adrenergic receptor, bradykinin receptor, etc.), ion channel proteins, intracellular attachment proteins, cellular adhesion proteins, and integrins. In certain aspects, the effected G-Protein Coupled Receptor (GPCR) interacts with a G protein α subunit (e.g., Gαₛ, Gαᵢ, Gα_{q}, and Gα₁₂).

In particular aspects, the electrokinetically altered aqueous fluids are suitable to modulate intracellular signal transduction, comprising modulation of a calcium dependant cellular messaging pathway or system (e.g., modulation of phospholipase C activity, or modulation of adenylate cyclase (AC) activity).

In particular aspects, the electrokinetically altered aqueous fluids are characterized by various biological activities (e.g., regulation of cytokines, receptors, enzymes and other proteins and intracellular signaling pathways) described herein.

In particular aspects, the electrokinetically altered aqueous fluids display synergy with Albuterol, and with Budesonide as shown herein

In particular aspects, the electrokinetically altered aqueous fluids reduce DEP-induced TSLP receptor expression in bronchial epithelial cells (BEC) as shown in working Examples herein.

In particular aspects, the electrokinetically altered aqueous fluids inhibit the DEP-induced cell surface-bound MMP9 levels in bronchial epithelial cells (BEC) as shown in working Examples herein.

In particular aspects, the biological effects of the electrokinetically altered aqueous fluids are inhibited by diphtheria toxin, indicating that beta blockade, GPCR blockade and Ca channel blockade affects the activity of the electrokinetically altered aqueous fluids (e.g., on regulatory T cell function) as shown herein.

In particular aspects, the physical and biological effects (e.g., the ability to alter cellular membrane structure or function sufficient to provide for modulation of intracellular signal transduction) of the electrokinetically altered aqueous fluids persists for at least two, at least three, at least four, at least five, at least 6 months, or longer periods, in a closed container (e.g., closed gas-tight container).

Therefore, further aspects provide said electrokinetically-generated solutions and methods of producing an electrokinetically altered oxygenated aqueous fluid or solution, comprising: providing a flow of a fluid material between two spaced surfaces in relative motion and defining a mixing volume therebetween, wherein the dwell time of a single pass of the flowing fluid material within and through the mixing volume is greater than 0.06 seconds or greater than 0.1 seconds; and introducing oxygen (O₂) into the flowing fluid material within the mixing volume under conditions suitable to dissolve at least 20 ppm, at least 25 ppm, at least 30, at least 40, at least 50, or at least 60 ppm oxygen into the material, and electrokinetically alter the fluid or solution. In certain aspects, the oxygen is infused into the material in less than 100 milliseconds, less than 200 milliseconds, less than 300 milliseconds, or less than 400 milliseconds. In particular embodiments, the ratio of surface area to the volume is at least 12, at least 20, at least 30, at least 40, or at least 50.

Yet further aspects, provide a method of producing an electrokinetically altered oxygenated aqueous fluid or solution, comprising: providing a flow of a fluid material between two spaced surfaces defining a mixing volume therebetween; and introducing oxygen into the flowing material within the mixing volume under conditions suitable to infuse at least 20 ppm, at least 25 ppm, at least 30, at least 40, at least 50, or at least 60 ppm oxygen into the material in less than 100 milliseconds, less than 200 milliseconds, less than 300 milliseconds, or less than 400 milliseconds. In certain aspects, the dwell time of the flowing material within the mixing volume is greater than 0.06 seconds or greater than 0.1 seconds. In particular embodiments, the ratio of surface area to the volume is at least 12, at least 20, at least 30, at least 40, or at least 50.

In particular aspects the administered inventive electrokinetically-altered fluids comprise charge-stabilized oxygen-containing nanostructures in an amount sufficient to provide modulation of at least one of cellular membrane potential and cellular membrane conductivity. In certain embodiments, the electrokinetically-altered fluids are superoxygenated (e.g., RNS-20, RNS-40 and RNS-60, comprising 20 ppm, 40 ppm and 60 ppm dissolved oxygen, respectively, in standard saline). In particular embodiments, the electrokinetically-altered fluids are not-superoxygenated (e.g., RNS-10 or Solas, comprising 10 ppm (e.g., approx. ambient levels of dissolved oxygen in standard saline). In certain aspects, the salinity, sterility, pH, etc., of the inventive electrokinetically-altered fluids is established at the time of electrokinetic production of the fluid, and the sterile fluids are administered by an appropriate route. Alternatively, at least one of the salinity, sterility, pH, etc., of the fluids is appropriately adjusted (e.g., using sterile saline or appropriate diluents) to be physiologically compatible with the route of administration prior to administration of the fluid. Preferably, and diluents and/or saline solutions and/or buffer compositions used to adjust at least one of the salinity, sterility, pH, etc., of the fluids are also electrokinetic fluids, or are otherwise compatible.

In particular aspects, the inventive electrokinetically-altered fluids comprise saline (e.g., one or more dissolved salt(s); e.g., alkali metal based salts (Li, Na, K, Rb, Cs, etc.), alkaline earth based salts (e.g., Mg, Ca), etc., transition metal-based salts (e.g., Cr, Fe, Co, Ni, Cu, Zn, etc.), along with any suitable anion/counterion components). Particular aspects comprise mixed salt based electrokinetic fluids (e.g., Na, K, Ca, Mg, etc., in various combinations and concentrations). In particular aspects, the inventive electrokinetically-altered fluids comprise standard saline (e.g., approx. 0.9% NaCl, or about 0.15 M NaCl). In particular aspects, the inventive electrokinetically-altered fluids comprise saline at a concentration of at least 0.0002 M, at least 0.0003 M, at least 0.001 M, at least 0.005 M, at least 0.01 M, at least 0.015 M, at least 0.1 M, at least 0.15 M, or at least 0.2 M. In particular aspects, the conductivity of the inventive electrokinetically-altered fluids is at least 10 μS/cm, at least 40 μS/cm, at least 80 μS/cm, at least 100 μS/cm, at least 150 μS/cm, at least 200 μS/cm, at least 300 μS/cm, or at least 500 μS/cm, at least 1 mS/cm, at least 5, mS/cm, 10 mS/cm, at least 40 mS/cm, at least 80 mS/cm, at least 100 mS/cm, at least 150 mS/cm, at least 200 mS/cm, at least 300 mS/cm, or at least 500 mS/cm. In particular aspects, any salt may be used in preparing the inventive electrokinetically-altered fluids, provided that they allow for formation of biologically active salt-stabilized nanostructures (e.g., salt-stabilized oxygen-containing nanostructures) as disclosed herein.

According to particular aspects, the biological effects of the inventive fluid compositions comprising charge-stabilized gas-containing nanostructures can be modulated (e.g., increased, decreased, tuned, etc.) by altering the ionic components of the fluids as, for example, described above, and/or by altering the gas component of the fluid. In preferred aspects, oxygen is used in preparing the inventive electrokinetic fluids. In additional aspects mixtures of oxygen along with at least one other gas selected from Nitrogen, Oxygen, Argon, Carbon dioxide, Neon, Helium, krypton, hydrogen and Xenon.

### Exemplary Relevant Molecular Interactions:

Conventionally, quantum properties are thought to belong to elementary particles of less than 10⁻¹⁰ meters, while the macroscopic world of our everyday life is referred to as classical, in that it behaves according to Newton's laws of motion.

Recently, molecules have been described as forming clusters that increase in size with dilution. These clusters measure several micrometers in diameter, and have been reported to increase in size non-linearly with dilution. Quantum coherent domains measuring 100 nanometers in diameter have been postulated to arise in pure water, and collective vibrations of water molecules in the coherent domain may eventually become phase locked to electromagnetic field fluctuations, providing for stable oscillations in water, providing a form of ‘memory’ in the form of excitation of long lasting coherent oscillations specific to dissolved substances in the water that change the collective structure of the water, which may in turn determine the specific coherent oscillations that develop. Where these oscillations become stabilized by magnetic field phase coupling, the water, upon dilution may still carry ‘seed’ coherent oscillations. As a cluster of molecules increases in size, its electromagnetic signature is correspondingly amplified, reinforcing the coherent oscillations carried by the water.

Despite variations in the cluster size of dissolved molecules and detailed microscopic structure of the water, a specificity of coherent oscillations may nonetheless exist. One model for considering changes in properties of water is based on considerations involved in crystallization.

A simplified protonated water cluster forming a nanoscale cage is shown in Applicants' previous patent application: WO 2009/055729. A protonated water cluster typically takes the form of H⁺(H₂0)ₙ. Some protonated water clusters occur naturally, such as in the ionosphere. Without being bound by any particular theory, and according to particular aspects, other types of water clusters or structures (clusters, nanocages, etc) are possible, including structures comprising oxygen and stabilized electrons imparted to the inventive output materials. Oxygen atoms may be caught in the resulting structures. The chemistry of the semi-bound nanocage allows the oxygen and/or stabilized electrons to remain dissolved for extended periods of time. Other atoms or molecules, such as medicinal compounds, can be caged for sustained delivery purposes. The specific chemistry of the solution material and dissolved compounds depend on the interactions of those materials.

Fluids processed by the mixing device have been shown previously via experiments to exhibit different structural characteristics that are consistent with an analysis of the fluid in the context of a cluster structure. See, for example, WO 2009/055729.

### Charge-Stabilized Nanostructures (e.g., Charge Stabilized Oxygen-Containing Nanostructures):

As described previously in Applicants' WO 2009/055729, “Double Layer Effect,” “Dwell Time,” “Rate of Infusion,” and “Bubble size Measurements,” the electrokinetic mixing device creates, in a matter of milliseconds, a unique non-linear fluid dynamic interaction of the first material and the second material with complex, dynamic turbulence providing complex mixing in contact with an effectively enormous surface area (including those of the device and of the exceptionally small gas bubbles of less that 100 nm) that provides for the novel electrokinetic effects described herein. Additionally, feature-localized electrokinetic effects (voltage/current) were demonstrated using a specially designed mixing device comprising insulated rotor and stator features.

As well-recognized in the art, charge redistributions and/or solvated electrons are known to be highly unstable in aqueous solution. According to particular aspects, Applicants' electrokinetic effects (e.g., charge redistributions, including, in particular aspects, solvated electrons) are surprisingly stabilized within the output material (e.g., saline solutions, ionic solutions). In fact, as described herein, the stability of the properties and biological activity of the inventive electrokinetic fluids (e.g., RNS-60 or Solas) can be maintained for months in a gas-tight container, indicating involvement of dissolved gas (e.g., oxygen) in helping to generate and/or maintain, and/or mediate the properties and activities of the inventive solutions. Significantly, the charge redistributions and/or solvated electrons are stably configured in the inventive electrokinetic ionic aqueous fluids in an amount sufficient to provide, upon contact with a living cell (e.g., mammalian cell) by the fluid, modulation of at least one of cellular membrane potential and cellular membrane conductivity (see, e.g., cellular patch clamp working Example 23 from WO 2009/055729 and as disclosed herein).

As described herein under “Molecular Interactions,” to account for the stability and biological compatibility of the inventive electrokinetic fluids (e.g., electrokinetic saline solutions), Applicants have proposed that interactions between the water molecules and the molecules of the substances (e.g., oxygen) dissolved in the water change the collective structure of the water and provide for nanoscale cage clusters, including nanostructures comprising oxygen and/or stabilized electrons imparted to the inventive output materials. Without being bound by mechanism, the configuration of the nanostructures in particular aspects is such that they: comprise (at least for formation and/or stability and/or biological activity) dissolved gas (e.g., oxygen); enable the electrokinetic fluids (e.g., RNS-60 or Solas saline fluids) to modulate (e.g., impart or receive) charges and/or charge effects upon contact with a cell membrane or related constituent thereof; and in particular aspects provide for stabilization (e.g., carrying, harboring, trapping) solvated electrons in a biologically-relevant form.

According to particular aspects, and as supported by the present disclosure, in ionic or saline (e.g., standard saline, NaCl) solutions, the inventive nanostructures comprise charge stabilized nanostructures (e.g., average diameter less that 100 nm) that may comprise at least one dissolved gas molecule (e.g., oxygen) within a charge-stabilized hydration shell. According to additional aspects, the charge-stabilized hydration shell may comprise a cage or void harboring the at least one dissolved gas molecule (e.g., oxygen). According to further aspects, by virtue of the provision of suitable charge-stabilized hydration shells, the charge-stabilized nanostructure and/or charge-stabilized oxygen containing nano-structures may additionally comprise a solvated electron (e.g., stabilized solvated electron).

Without being bound by mechanism or particular theory, after the present priority date, charge-stabilized microbubbles stabilized by ions in aqueous liquid in equilibrium with ambient (atmospheric) gas have been proposed (Bunkin et al., Journal of Experimental and Theoretical Physics, 104:486-498, 2007; incorporated herein by reference in its entirety). According to particular aspects of the present invention, Applicants' novel electrokinetic fluids comprise a novel, biologically active form of charge-stabilized oxygen-containing nanostructures, and may further comprise novel arrays, clusters or associations of such structures.

According to the charge-stabilized microbubble model, the short-range molecular order of the water structure is destroyed by the presence of a gas molecule (e.g., a dissolved gas molecule initially complexed with a nonadsorptive ion provides a short-range order defect), providing for condensation of ionic droplets, wherein the defect is surrounded by first and second coordination spheres of water molecules, which are alternately filled by adsorptive ions (e.g., acquisition of a ‘screening shell of Na⁺ ions to form an electrical double layer) and nonadsorptive ions (e.g., Cl⁻ ions occupying the second coordination sphere) occupying six and 12 vacancies, respectively, in the coordination spheres. In under-saturated ionic solutions (e.g., undersaturated saline solutions), this hydrated ‘nucleus’ remains stable until the first and second spheres are filled by six adsorptive and five nonadsorptive ions, respectively, and then undergoes Coulomb explosion creating an internal void containing the gas molecule, wherein the adsorptive ions (e.g., Na⁺ ions) are adsorbed to the surface of the resulting void, while the nonadsorptive ions (or some portion thereof) diffuse into the solution (Bunkin et al., supra). In this model, the void in the nanostructure is prevented from collapsing by Coulombic repulsion between the ions (e.g., Na⁺ ions) adsorbed to its surface. The stability of the void-containing nanostructures is postulated to be due to the selective adsorption of dissolved ions with like charges onto the void/bubble surface and diffusive equilibrium between the dissolved gas and the gas inside the bubble, where the negative (outward electrostatic pressure exerted by the resulting electrical double layer provides stable compensation for surface tension, and the gas pressure inside the bubble is balanced by the ambient pressure. According to the model, formation of such microbubbles requires an ionic component, and in certain aspects collision-mediated associations between particles may provide for formation of larger order clusters (arrays) (Id).

The charge-stabilized microbubble model suggests that the particles can be gas microbubbles, but contemplates only spontaneous formation of such structures in ionic solution in equilibrium with ambient air, is uncharacterized and silent as to whether oxygen is capable of forming such structures, and is likewise silent as to whether solvated electrons might be associated and/or stabilized by such structures.

According to particular aspects, the inventive electrokinetic fluids comprising charge-stabilized nanostructures and/or charge-stabilized oxygen-containing nanostructures are novel and fundamentally distinct from the postulated non-electrokinetic, atmospheric charge-stabilized microbubble structures according to the microbubble model. Significantly, this conclusion is unavoidable, deriving, at least in part, from the fact that control saline solutions do not have the biological properties disclosed herein, whereas Applicants' charge-stabilized nanostructures provide a novel, biologically active form of charge-stabilized oxygen-containing nanostructures.

According to particular aspects of the present invention, Applicants' novel electrokinetic device and methods provide for novel electrokinetically-altered fluids comprising significant quantities of charge-stabilized nanostructures in excess of any amount that may or may not spontaneously occur in ionic fluids in equilibrium with air, or in any non-electrokinetically generated fluids. In particular aspects, the charge-stabilized nanostructures comprise charge-stabilized oxygen-containing nanostructures. In additional aspects, the charge-stabilized nanostructures are all, or substantially all charge-stabilized oxygen-containing nanostructures, or the charge-stabilized oxygen-containing nanostructures the major charge-stabilized gas-containing nanostructure species in the electrokinetic fluid.

According to yet further aspects, the charge-stabilized nanostructures and/or the charge-stabilized oxygen-containing nanostructures may comprise or harbor a solvated electron, and thereby provide a novel stabilized solvated electron carrier. In particular aspects, the charge-stabilized nanostructures and/or the charge-stabilized oxygen-containing nanostructures provide a novel type of electride (or inverted electride), which in contrast to conventional solute electrides having a single organically coordinated cation, rather have a plurality of cations stably arrayed about a void or a void containing an oxygen atom, wherein the arrayed sodium ions are coordinated by water hydration shells, rather than by organic molecules. According to particular aspects, a solvated electron may be accommodated by the hydration shell of water molecules, or preferably accommodated within the nanostructure void distributed over all the cations. In certain aspects, the inventive nanostructures provide a novel ‘super electride’ structure in solution by not only providing for distribution/stabilization of the solvated electron over multiple arrayed sodium cations, but also providing for association or partial association of the solvated electron with the caged oxygen molecule(s) in the void—the solvated electron distributing over an array of sodium atoms and at least one oxygen atom. According to particular aspects, therefore, ‘solvated electrons’ as presently disclosed in association with the inventive electrokinetic fluids, may not be solvated in the traditional model comprising direct hydration by water molecules. Alternatively, in limited analogy with dried electride salts, solvated electrons in the inventive electrokinetic fluids may be distributed over multiple charge-stabilized nanostructures to provide a ‘lattice glue’ to stabilize higher order arrays in aqueous solution.

In particular aspects, the inventive charge-stabilized nanostructures and/or the charge-stabilized oxygen-containing nanostructures are capable of interacting with cellular membranes or constituents thereof, or proteins, etc., to mediate biological activities. In particular aspects, the inventive charge-stabilized nanostructures and/or the charge-stabilized oxygen-containing nanostructures harboring a solvated electron are capable of interacting with cellular membranes or constituents thereof, or proteins, etc., to mediate biological activities.

In particular aspects, the inventive charge-stabilized nanostructures and/or the charge-stabilized oxygen-containing nanostructures interact with cellular membranes or constituents thereof, or proteins, etc., as a charge and/or charge effect donor (delivery) and/or as a charge and/or charge effect recipient to mediate biological activities. In particular aspects, the inventive charge-stabilized nanostructures and/or the charge-stabilized oxygen-containing nanostructures harboring a solvated electron interact with cellular membranes as a charge and/or charge effect donor and/or as a charge and/or charge effect recipient to mediate biological activities.

In particular aspects, the inventive charge-stabilized nanostructures and/or the charge-stabilized oxygen-containing nanostructures are consistent with, and account for the observed stability and biological properties of the inventive electrokinetic fluids, and further provide a novel electride (or inverted electride) that provides for stabilized solvated electrons in aqueous ionic solutions (e.g., saline solutions, NaCl, etc.).

In particular aspects, the charge-stabilized oxygen-containing nanostructures substantially comprise, take the form of, or can give rise to, charge-stabilized oxygen-containing nanobubbles. In particular aspects, charge-stabilized oxygen-containing clusters provide for formation of relatively larger arrays of charge-stabilized oxygen-containing nanostructures, and/or charge-stabilized oxygen-containing nanobubbles or arrays thereof. In particular aspects, the charge-stabilized oxygen-containing nanostructures can provide for formation of hydrophobic nanobubbles upon contact with a hydrophobic surface.

In particular aspects, the charge-stabilized oxygen-containing nanostructures substantially comprise at least one oxygen molecule. In certain aspects, the charge-stabilized oxygen-containing nanostructures substantially comprise at least 1, at least 2, at least 3, at least 4, at least 5, at least 10 at least 15, at least 20, at least 50, at least 100, or greater oxygen molecules. In particular aspects, charge-stabilized oxygen-containing nanostructures comprise or give rise to nanobubbles (e.g., hydrophobid nanobubbles) of about 20 nm×1.5 nm, comprise about 12 oxygen molecules (e.g., based on the size of an oxygen molecule (approx 0.3 nm by 0.4 nm), assumption of an ideal gas and application of n=PV/RT, where P=1 atm, R=0.082 057 l.atm/mol.K; T=295K; V=pr²h=4.7×10⁻²² L, where r=10×10⁻⁹ m, h=1.5×10⁻⁹ m, and n=1.95×10⁻²² moles).

In certain aspects, the percentage of oxygen molecules present in the fluid that are in such nanostructures, or arrays thereof, having a charge-stabilized configuration in the ionic aqueous fluid is a percentage amount selected from the group consisting of greater than: 0.1%, 1%; 2%; 5%; 10%; 15%; 20%; 25%; 30%; 35%; 40%; 45%; 50%; 55%; 60%; 65%; 70%; 75%; 80%; 85%; 90%; and greater than 95%. Preferably, this percentage is greater than about 5%, greater than about 10%, greater than about 15%, or greater than about 20%. In additional aspects, the substantial size of the charge-stabilized oxygen-containing nanostructures, or arrays thereof, having a charge-stabilized configuration in the ionic aqueous fluid is a size selected from the group consisting of less than: 100 nm; 90 nm; 80 nm; 70 nm; 60 nm; 50 nm; 40 nm; 30 nm; 20 nm; 10 nm; 5 nm; 4 nm; 3 nm; 2 nm; and 1 nm. Preferably, this size is less than about 50 nm, less than about 40 nm, less than about 30 nm, less than about 20 nm, or less than about 10 nm.

In certain aspects, the inventive electrokinetic fluids comprise solvated electrons. In further aspects, the inventive electrokinetic fluids comprises charge-stabilized nanostructures and/or charge-stabilized oxygen-containing nanostructures, and/or arrays thereof, which comprise at least one of: solvated electron(s); and unique charge distributions (polar, symmetric, asymmetric charge distribution). In certain aspects, the charge-stabilized nanostructures and/or charge-stabilized oxygen-containing nanostructures, and/or arrays thereof, have paramagnetic properties.

By contrast, relative to the inventive electrokinetic fluids, control pressure pot oxygenated fluids (non-electrokinetic fluids) and the like do not comprise such electrokinetically generated charge-stabilized biologically-active nanostructures and/or biologically-active charge-stabilized oxygen-containing nanostructures and/or arrays thereof, capable of modulation of at least one of cellular membrane potential and cellular membrane conductivity.

### Systems for Making Gas-Enriched Fluids

The presently disclosed system and methods allow gas (e.g. oxygen) to be enriched stably at a high concentration with minimal passive loss. This system and methods can be effectively used to enrich a wide variety of gases at heightened percentages into a wide variety of fluids. By way of example only, deionized water at room temperature that typically has levels of about 2-3 ppm (parts per million) of dissolved oxygen can achieve levels of dissolved oxygen ranging from at least about 5 ppm, at least about 10 ppm, at least about 15 ppm, at least about 20 ppm, at least about 25 ppm, at least about 30 ppm, at least about 35 ppm, at least about 40 ppm, at least about 45 ppm, at least about 50 ppm, at least about 55 ppm, at least about 60 ppm, at least about 65 ppm, at least about 70 ppm, at least about 75 ppm, at least about 80 ppm, at least about 85 ppm, at least about 90 ppm, at least about 95 ppm, at least about 100 ppm, or any value greater or therebetween using the disclosed systems and/or methods. In accordance with a particular exemplary embodiment, oxygen-enriched water may be generated with levels of about 30-60 ppm of dissolved oxygen.

Table 1 illustrates various partial pressure measurements taken in a healing wound treated with an oxygen-enriched saline solution (Table 1) and in samples of the gas-enriched oxygen-enriched saline solution of the present invention.

TABLE 1
TISSUE OXYGEN MEASUREMENTS
Probe Z082BO
In air: 171 mmHg 23° C.

| Column | Partial Pressure (mmHg) |
|---|---|
| B1 | 32-36 |
| B2 | 169-200 |
| B3 | 20-180* |
| B4 | 40-60 |

*wound depth minimal, majority >150, occasional 20 s

### MMP-9 Mediated Conditions

MMP-9 is believed to be involved in many types of diseases, disorders, and/or conditions including, but not limited to, several types of cancers (e.g. breast cancer, gastric cancer, endometrial carcinoma, glioblastomas, and primary central nervous system lymphoma (PCNSL)), cardiovascular diseases (e.g. atherosclerosis and restenosis), neuropsychiatric disorders (e.g. schizophrenia and bipolar illness), pulmonary diseases (e.g. asthma and chronic bronchitis), neuroinflammatory degenerative diseases (e.g. Alzheimer's disease, Parkinson's disease, Huntington's disease, amyotrophic lateral sclerosis (ALS, also known as Lou Gehrig's disease), and diabetic retinopathy), autoimmune diseases (e.g. multiple sclerosis, lupus erythematosus, and rheumatoid arthritis), and nervous system-related disorders and conditions (e.g. stroke/cerebral ischemia, head trauma, spinal cord injury, migraine, cerebral amyloid angiopathy, HIV-associated dementia (AIDS), age-related cognitive decline; mild cognitive impairment, and prion diseases in a mammal).

### Indications

Wound healing. MMP-9 has been shown to have a role in scar-free wound healing in nude mice (Manuel and Gawronska-Kozak, Matrix Biology, 25:505-514, 2006). In particular, Manuel and Gawronska-Kozak showed that post-injured skin tissue had high levels of MMP-9 mRNA and protein when compared to wildtype mice during the remodeling phase of wound healing. Another study found that MMP-9 was highly expressed in human oral mucosa post wounding. Since wound healing requires keratinocyte migration and granulation tissue remodeling, the study thus indicated that in wound healing, MMP-9 is involved in keratinocyte migration and granulation tissue remodeling (Salo et al., Lab Invest. 70:176-82, 1994). These results indicate that MMP-9 plays an important role in wound healing. However, a more recent study demonstrated that prolonged MMP-9 production resulted in poor wound healing in ulcers from diabetic patients, due at least in part to prolonged inflammation (Liu et al., Diabetes Care, 32:117-119, 2009). This result indicates that MMP-9, while necessary during the remodeling phase of wound healing, inhibits full healing of ulcers. Applicants show herein, using a BEC model, that the inventive electrokinetically-altered fluids significantly downregulated production of MMP-9. According to certain embodiments, the inventive electrokinetically-altered fluids have substantial utility for treating wounds and similar conditions.

Cancers. A recent review describes the role of MMP-9 in different cancers (Rybakowski, Cardiovascular Psychiatry and Neurology, Vol. 2009, Article ID 904836, 7 pages). More specifically, the review discusses how a genetic mutation that increases expression of MMP-9 mRNA is frequently associated with an increased risk of some kinds of cancer and with more severe progression of the tumor and/or greater dynamics of metastases. In addition, several studies have shown that this particular genetic mutation was associate with malignancy, growth of tumors and the severity of lymph node metastases in colorectal cancer, breast cancer, gastric cancer, and urinary bladder cancer. In addition, a recent study examining the level of MMP-9 in tumors from gastric carcinomas compared to normal gastric mucosa demonstrated that the carcinomas had a significantly increased level of MMP-9 protein and that increase was significantly associated with worse survival of the patients (Sier et al., J. Thrombosis and Haemostasis, 4:127-127, 2006). Another study demonstrated that MMP-9 mRNA and MMP-9 activity were higher in gliomas than in normal brain and more strongly correlated with severity of tumor (Forsyth, et al., British J. Cancer, 79:1828-1835, 1999). These studies indicate that MMP-9 plays an important role in the pathogenesis of many types of cancers. Applicants show herein, using a BEC model, that the inventive electrokinetically-altered fluids significantly downregulated production of MMP-9. According to certain embodiments, the inventive electrokinetically-altered fluids have substantial utility for treating certain cancers (e.g. colorectal cancer, breast cancer, gastric cancer, gliomas and urinary bladder cancer) and similar conditions and limiting lymph node metastases and alleviating complications relating to cancerous conditions.

Pulmonary diseases (e.g. asthma and chronic bronchitis). Recently, it has been demonstrated that the levels of MMP-9 are significantly increased in patients with stable asthma and even higher in patients with acute asthmatic patients compared with healthy control subjects. MMP-9 plays a crucial role in the infiltration of airway inflammatory cells and the induction of airway hyper-responsiveness indicating that MMP-9 may have an important role in inducing and maintaining asthma (Vignola et al., Sputum metalloproteinase-9/tissue inhibitor of metalloproteinase-1 ratio correlates with airflow obstruction in asthma and chronic bronchitis, Am J Respir Crit Care Med 158:1945-1950, 1998; Hoshino et al., Inhaled corticosteroids decrease subepithelial collagen deposition by modulation of the balance between matrix metalloproteinase-9 and tissue inhibitor of metalloproteinase-1 expression in asthma, J Allergy Clin Immunol 104:356-363, 1999; Simpson et al., Differential proteolytic enzyme activity in eosinophilic and neutrophilic asthma, Am J Respir Crit Care Med 172:559-565, 2005; Lee et al., A murine model of toluene diisocyanate-induced asthma can be treated with matrix metalloproteinase inhibitor, J Allergy Clin Immunol 108:1021-1026, 2001; and Lee et al., Matrix metalloproteinase inhibitor regulates inflammatory cell migration by reducing ICAM-1 and VCAM-1 expression in a murine model of toluene diisocyanate-induced asthma, J Allergy Clin Immunol 2003; 111:1278-1284). In addition, a recent study demonstrated that apical treatment using MMP-9 resulted in 1) decreased immunostaining of proteins found in tight junctions and 2) increased infection efficiency by virus (e.g. viral access to epithelial basolateral surface), both of which indicate disruption of barrier function (Vermeer et al., Am J Physiol Lung Cell Mol Physiol, 296:L751-L762, 2009). These studies indicate that MMP-9 plays an important role in the pathogenesis of pulmonary diseases (e.g. asthma and chronic bronchitis). Applicants show herein, using a BEC model, that the inventive electrokinetically-altered fluids significantly downregulated production of MMP-9. According to certain embodiments, the inventive electrokinetically-altered fluids have substantial utility for treating pulmonary diseases (e.g. asthma and chronic bronchitis) and similar conditions.

Cardiovascular diseases. A recent review describes the role of MMP-9 in cardiovascular diseases (e.g. CHD, atherosclerosis, and hypertension) (Rybakowski, 2009). More specifically, the review discusses how the genetic mutation that increases expression of MMP-9 mRNA is related to an increased progression and mortality of coronary heart disease (CHD), increased atherosclerosis, and increased progression of hypertension. In further studies correlations between higher MMP-9 levels and coronary artery ectasia, higher MMP-9 levels and hypertrophic cardiomyopathy, and higher MMP-9 levels and hypertensive patients were found. These studies indicate that MMP-9 plays an important role in the pathogenesis of cardiovascular diseases (e.g. CHD, atherosclerosis, and hypertension). Applicants show herein, using a BEC model, that the inventive electrokinetically-altered fluids significantly downregulated production of MMP-9. According to certain embodiments, the inventive electrokinetically-altered fluids have substantial utility for treating cardiovascular diseases (e.g. CHD, atherosclerosis, and hypertension) and similar conditions.

Neuropsychiatric disorders. A recent review describes the role of MMP-9 in neuropsychiatric disorders (e.g. schizophrenia and bipolar mood disorder) (Rybakowski, 2009). More specifically, the review discusses how patients with bipolar mood disorder had a significant prevalence of the genetic mutation that increases expression of MMP-9 mRNA when compared to healthy control subjects. However, schizophrenia patients when compared to normal patients had a significantly lower association of the genetic mutation that increases expression of MMP-9 mRNA. These studies indicate that MMP-9 has a correlative role in neuropsychiatric disorders (e.g. schizophrenia and bipolar mood disorder). Applicants show herein, using a BEC model, that the inventive electrokinetically-altered fluids significantly downregulated production of MMP-9. According to certain embodiments, the inventive electrokinetically-altered fluids have substantial utility for treating neuropsychiatric disorders (e.g. schizophrenia and bipolar mood disorder) and similar conditions.

Neuroinflammatory degenerative diseases. For several years MMP-9 has been implicated in the pathogenesis of diseases like Alzheimer's disease and ALS. Increased expression of MMP-9 also has been reported in postmortem Alzheimer's disease and ALS brain tissue (Lorenzl et al., Neurochem. Int. 43:191-6, 2003). Lorenzl et al. also reported increased levels of MMP-9 circulating in plasma in Alzheimer's patients. Interestingly, a recent study demonstrated that MMP-9 can degrade tightly aggregated amyloid-beta fibrils and may contribute to ongoing clearance of plaques from amyloid-laden brains (Yan et al., J Biol Chem. 281:24566-74, 2006). Another recent study showed increased expression of MMP-9 in the cortex and cerebellum of post-mortem Huntington's disease patient samples compared to controls (Silverstroni, et al., Clinical Neuroscience and Neuropathology, 20:1098-1103, 2009). These studies indicate that MMP-9 has an important role in neuroinflammatory degenerative diseases (e.g. Alzheimer's disease, Huntington's disease and ALS). Applicants show herein, using a BEC model, that the inventive electrokinetically-altered fluids significantly downregulated production of MMP-9. According to certain embodiments, the inventive electrokinetically-altered fluids have substantial utility for treating neuroinflammatory degenerative diseases (e.g. Alzheimer's disease, Huntington's disease and ALS) and similar conditions.

HIV-associated dementia (AIDS). MMP-9 is involved with degrading the extracellular matrix and leads to reduced fortitude of the blood-brain barrier, which may lead to blood-brain barrier dysfunction if MMP-9 is too active. According to certain embodiments, the inventive electrokinetically-altered fluids significantly downregulate production of MMP-9 (see herein below) and can thus increase the resilience of the blood-brain barrier thereby reducing HIV infection of the brain and subsequent dementia.

Autoimmune disorders. For several years, studies have demonstrated the presence or increased presence of MMP-9 in bodily fluids from many autoimmune disorders including but not limited to multiple sclerosis, SLE, and rheumatoid arthritis. A recent review discussed and summarized studies that examined the presence and amount of MMP-9 in serum, cerebrospinal fluid (CSF), and synovial fluid from patients afflicted with multiple sclerosis, SLE, and rheumatoid arthritis (Ram et al., J. Clinical Immunology, 26:299-307, 2006). In general, each study found an increase in MMP-9 in the respective body fluid when compared to controls. In another study, Ainiala, et al., discovered a correlation between increased serum MMP-9 and neuropsychiatric manifestations (e.g. patients having at least one neuropsychiatric symptom, like cognitive dysfunction), small-vessel cerebral vasculopathy, and an increased risk of cerebral ischemic events in patients with SLE (Ainiala, et al., Arthritis Rheum 50:858-65, 2004).

For rheumatoid arthritis, a study has demonstrated substantial elevation of MMP-9 levels in patient sera and synovial fluid (Gruber et al., Clin Immunol Immunopathol, 78:161-71, 1996). The study also discovered that the source of MMP-9 in rheumatoid arthritis is from rheumatoid arthritis synovium. The in situ reverse transcriptase PCR found that the MMP-9 transcript was produced in several different types of rheumatoid synovial cells. In another study, MMP-9 levels were found to be substantially higher in joint fluids from rheumatoid arthritis when compared to joint fluids from osteoarthritis (Seki, et al., Modern Rheumatology, 7:197-209, 2009). In addition, the study demonstrated that active MMP-9 concentrations in joint fluids from rheumatoid arthritis patients was positively correlated with the MMP-9 positive cells in rheumatoid arthritis synovium and to the score of diffuse infiltrates of lymphocytes. These results indicate that active MMP-9 actively participates in joint destruction of rheumatoid arthritis.

For multiple sclerosis, studies have shown that MMP-9 predominates in multiple sclerosis acute lesions. Several studies have also demonstrated that the levels of MMP-9 in sera and CSF are different depending on which type of the disease the patient has. In particular, one study showed that short disease duration and relapsing-remitting multiple sclerosis had much higher levels of MMP-9 than primary progressive multiple sclerosis (Avolio, et al., J Neuroimmunol, 136:46-53, 2003). However, the primary progressive multiple sclerosis had much higher levels of MMP-9 than a patient with inactive multiple sclerosis. A recent study examined the effects of estriol, a pregnancy hormone having anti-inflammatory properties, on the production of MMP-9 from peripheral blood mononuclear cells (PBMCs) collected from female multiple sclerosis patients (Gold, et al., Lab Investigation 89:1076-83, 2009). Gold, et al., demonstrated that production of MMP-9 was significantly decreased in PBMCs from those patients having the relapsing-remitting type of multiple sclerosis. Interestingly, this decrease of MMP-9 correlated with a decrease in enhancing lesions on MRI and a decrease in T cell and macrophage infiltration into the central nervous system as shown in the EAE mouse model.

These studies indicate that MMP-9 has an important role in autoimmune disorders (e.g. multiple sclerosis, SLE, and rheumatoid arthritis). Applicants show herein, using a BEC model, that the inventive electrokinetically-altered fluids significantly downregulated production of MMP-9. According to certain embodiments, the inventive electrokinetically-altered fluids have substantial utility for treating autoimmune disorders (e.g. multiple sclerosis, SLE, and rheumatoid arthritis) and similar conditions.

Migraine. Recently a study has shown an increased production of MMP-9 in migraine patients during migraine attacks, indicating that a migraine attack has an inflammation and/or blood-brain barrier disruption component. These studies indicate that MMP-9 has a role in migraine attacks. Applicants show herein, using a BEC model, that the inventive electrokinetically-altered fluids significantly downregulated production of MMP-9. According to certain embodiments, the inventive electrokinetically-altered fluids have substantial utility for treating migraine attacks and similar conditions.

Stroke. MMP-9 involvement in remodeling extracellular matrix and more specifically degrading the blood-brain barrier has been long appreciated in the art. MMP-9 role in post-stroke recovery has been examined (Clark, et al., Neuroscience Letters, 238:53-56, 1997). Clark, et al., demonstrated that MMP-9 activity is markedly elevated in the infarcted tissue two days post-infarction. Interestingly, those patients that died from the stroke had elevated levels of active MMP-9 starting from 2 days and lasting until death, indicating that the prolonged active MMP-9 has a role in morality resulting from a stroke. These studies indicate that MMP-9 has a role in stroke recovery. Applicants show herein, using a BEC model, that the inventive electrokinetically-altered fluids significantly downregulated production of MMP-9. According to certain embodiments, the inventive electrokinetically-altered fluids have substantial utility for treating patient recovering from a stroke and similar conditions.

### MMP Inhibitors:

A number of metalloproteinase inhibitors are known (see, for example, the reviews of MMP inhibitors by Beckett R. P. and Whittaker M., 1998, Exp. Opin. Ther. Patents, 8(3):259-282; and by Whittaker M. et al, 1999, Chemical Reviews 99(9):2735-2776). WO 02/074767 discloses hydantoin derivatives of formula that are useful as MMP inhibitors, particularly as potent MMP12 inhibitors. U.S. patent application Ser. No. 11/721,590 (published as 20080032997) discloses a further group of hydantoin derivatives that are inhibitors of metalloproteinases and are of particular interest in inhibiting MMPs such as MMP12 and MMP9. Novel triazolone derivatives for inhibiting MMPs such as MMP12 and MMP9 are disclosed in U.S. patent application Ser. No. 10/593,543 (published as 20070219217). Additional MMP12 and MMP9 inhibitors are disclosed in Ser. No. 11/509,490 (published as 20060287338) (see also Ser. No. 10/831,265 (published as 20040259896)).

Additional exemplary MMP inhibitors are summarize in Table 2 below:

TABLE 2
Exemplary Matrix Metalloproteinase (MMP) Inhibitors (e.g., obtainable from EMD Biosciences).

| Product/ | Cat. | | |
|---|---|---|---|
| Identifier | No. | Comment | Structure |
| Chlorhexidine, Dihydrochloride | 220557 | Acts as a Zn2+−chelating inhibitor of MMP-2 and MMP-9. | 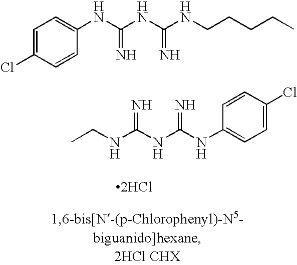 |
| CL 82198 | 233105 | A selective MMP-13 inhibitor (IC₅₀ = | |
| | | 10 μM). Does not inhibit MMP-1, | |
| | | MMP-9, and TACE. | |
| GM 1489 | 364200 | Kᵢ = 200 pM for MMP-1, 500 nM for MMP-2, 20 μM for MMP-3, 100 nM for MMP-8, and 100 nM for MMP-9 | 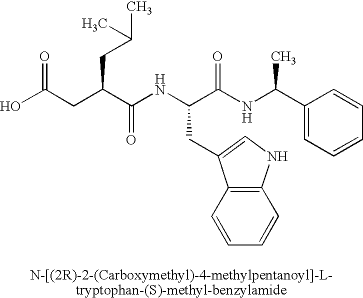 |
| GM 6001 (Galardin) | 364205 | Kᵢ = 400 pM for MMP-1, 500 pM for MMP-2, 27 nM for MMP-3, 100 pM for MMP-8, and 200 pM for MMP-9. See also Cat. No. 364206. | 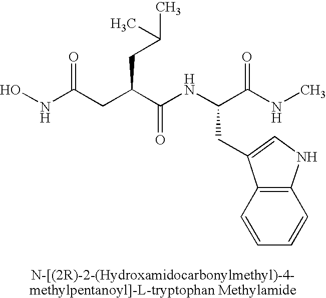 |
| GM 6001, Negative Control | 364210 | Useful negative control for GM 6001 | 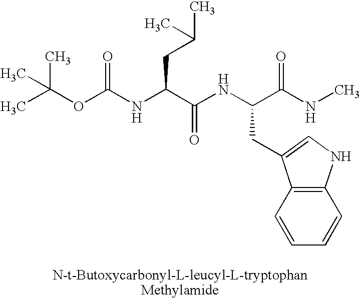 |
| MMP Inhibitor I | 444250 | IC₅₀ = 1.0 μM for MMP-1 and MMP-8; | 4-Abz-Gly-Pro-D-Leu-D-Ala-NH-OH[Abz = |
| (FN-439) | | IC₅₀ = 30 μM for MMP-9; IC₅₀ = 150 | aminobenzoyl] |
| | | μM for MMP-3 | |
| MMP Inhibitor II | 444247 | IC₅₀ = 24 nM for MMP-1, 18.4 nM for MMP-3, 30 nM for MMP-7, and 2.7 nM for MMP-9. | 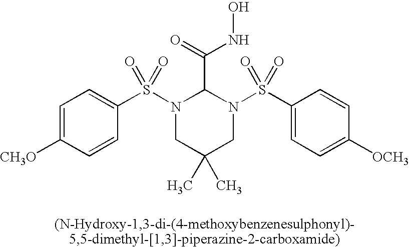 |
| MMP Inhibitor III | 444264 | A broad-spectrum MMP inhibitor. IC₅₀ = 7.4 nM for MMP-1, 2.3 nM for MMP-2, 135 nM for MMP-3, 10-100 nM for MMP-7, and 1-10 nM for MMP-13. | 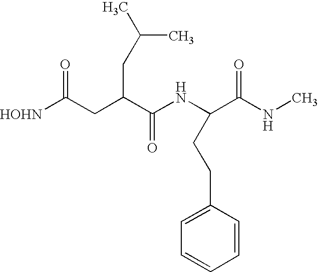 |
| MMP Inhibitor IV | 444271 | A peptide hydroxamic acid that | HONH—COCH₂CH₂CO-Phe-Ala-NH₂ |
| | | potently inhibits MMPs and | |
| | | pseudolysin from P. aeruginosa. | |
| MMP-2 Inhibitor I (OA-Hy) | 444244 | Kᵢ = 1.7 μM | 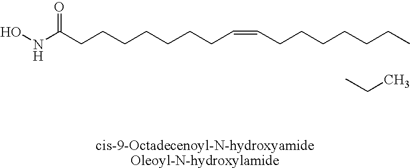 |
| MMP-2/MMP-3 Inhibitor I | 444239 | Kᵢ = 17 μM for MMP-2 and 290 nM for MMP-3. | 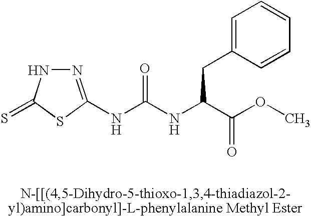 |
| MMP-2/MMP-3 Inhibitor II | 444240 | Kᵢ = 1.5 μM for MMP-2 and 520 nM for MMP-3 | 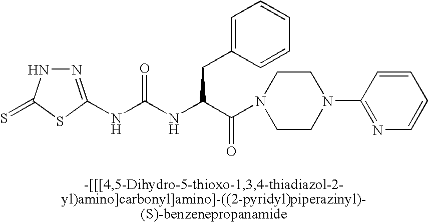 |
| MMP-2/MMP-9 Inhibitor I | 444241 | IC₅₀ = 310 nM for MMP-2 and 240 nM for MMP-9 | 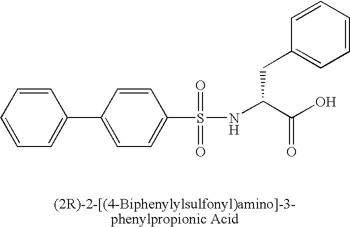 |
| MMP-2/MMP-9 Inhibitor II | 444249 | IC₅₀ = 17 nM for MMP-2 and 30 nM for MMP-9 | 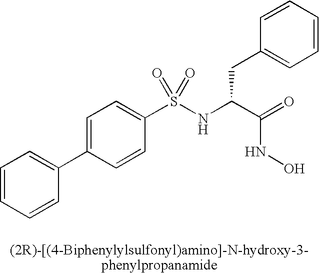 |
| MMP-2/MMP-9 | 444251 | IC₅₀ = 10 μM for MMP-2 and 10 M | H-Cys1-Thr-Thr-His-Trp-Gly-Phe-Thr-Leu-Cys10- |
| Inhibitor III | | for MMP-9 | OH (cyclic: 1 → 10) |
| MMP-2/MMP-9 | 444274 | A slow-binding and irreversible | HONH—COCH₂CH₂CO-FA-NH₂ |
| Inhibitor IV | | inhibitor of MMP-2 (Kᵢ = 13.9 nM) | |
| | | and MMP-9 (Kᵢ = 600 nM). | |
| MMP-3 Inhibitor I | 444218 | IC₅₀ = 5 μM | Ac-Arg-Cys-Gly-Val-Pro-Asp-NH₂ |
| MMP-3 Inhibitor II | 444225 | Kᵢ = 130 nM | 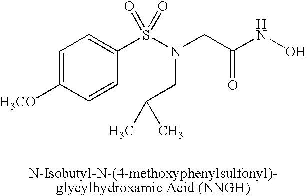 |
| MMP-3 Inhibitor III | 444242 | Kᵢ = 3.2 μM | 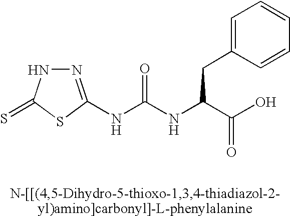 |
| MMP-3 Inhibitor IV | 444243 | Kᵢ = 810 nM | 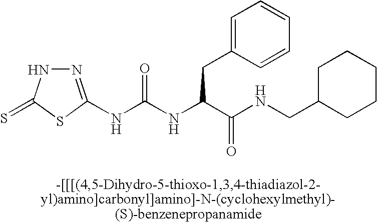 |
| MMP-3 Inhibitor V | 444260 | A potent and competitive inhibitor of | 4-Dibenzofuran-2′-yl-4-hydroximino-butyric Acid |
| | | both human and rabbit MMP-3 | |
| | | catalytic domains with Kᵢ values in the | |
| | | low μM range. | |
| MMP-3 Inhibitor | 444265 | A potent and competitive inhibitor of | 4-(4′-Biphenyl)-4-hydroxyimino-butyric Acid |
| VI | | both human and rabbit MMP-3 | |
| | | catalytic domains with Kᵢ values in the | |
| | | low μM range. | |
| MMP-3 Inhibitor VII | 444280 | A potent nonpeptide inhibitor of MMP- 3 (IC₅₀ = 25 nM against the catalytic domain). | 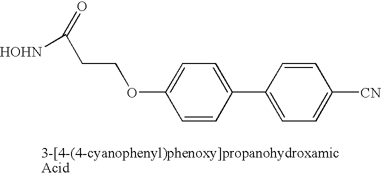 |
| MMP-3 Inhibitor VIII | 444281 | A cell-permeable, potent inhibitor of human MMP-3 (Kᵢ = 32 nM) and murine macrophage metalloelastase (MME/MMP-12; IC₅₀ = 13 nM). | 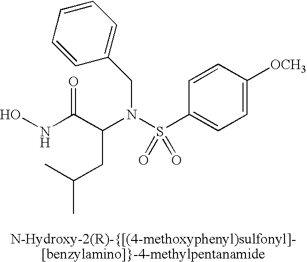 |
| MMP-8 Inhibitor I | 444237 | IC₅₀ = 4 nM | 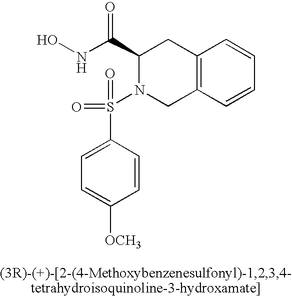 |
| MMP-8 Inhibitor I, Negative Control | 444238 | Useful negative control for MMP-8 Inhibitor I (IC₅₀ = 1000 nM). | 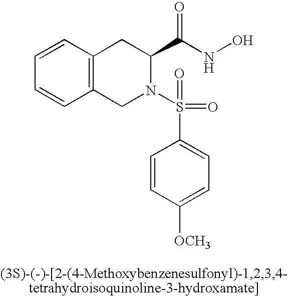 |
| MMP-9 Inhibitor I | 444278 | A potent and selective inhibitor of | Structure not available |
| | | MMP-9 (IC₅₀ = 5 nM). Also inhibits | |
| | | MMP-1 (IC₅₀ = 1.05 μM) and MMP-13 | |
| | | (IC₅₀ = 113 nM). | |
| MMP-9/MMP-13 Inhibitor I | 444252 | IC₅₀ = 900 pM for MMP-9 and 900 pM for MMP-13, Also inhibits MMP-1 (IC₅₀ = 43 nM), MMP-3 (IC₅₀ = 23 nM). and MMP-7 (IC₅₀ = 930 nM). | 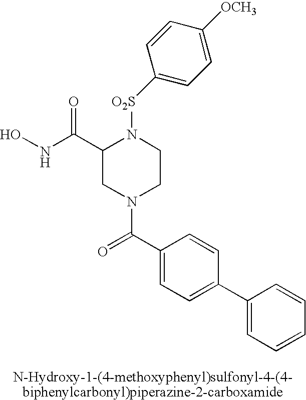 |
| MMP-9/MMP-13 Inhibitor II | 444253 | IC₅₀ = 1.9 nM for MMP-9 and 1.3 nM for MMP-13. Also inhibits MMP-1 (IC₅₀ = 24 nM), MMP-3 (IC₅₀ = 18 nM), and MMP-7 (IC₅₀ = 230 nM). | 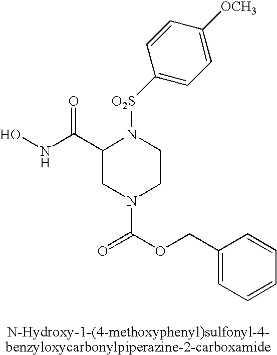 |
| Trocade | | See Marion Flipo et al., “A library of novel hydroxamic acids targeting the metallo-protease family: Design, parallel synthesis and screening,” Bioorganic & Medicinal Chemistry 15, pp. 63-76 (2007) incorporated herein by reference in its entirety. | 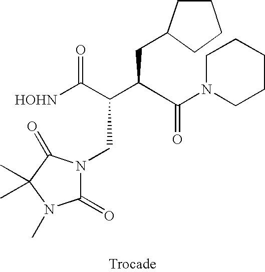 |
| Marimastat | | See Marion Flipo et al. supra. | 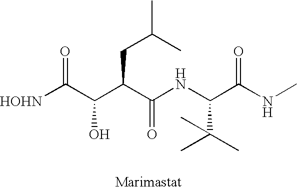 |
| CGS-27023 | | See Marion Flip et al. supra. | 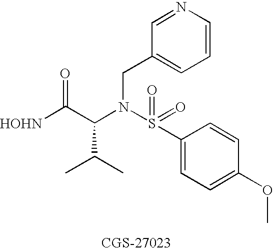 |
| SAHA | | See Marion Flipo et al. supra. | 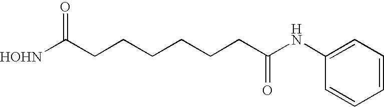 |
| Prinomastat (AG-3340) | | See Marion Flipo et al. supra. | 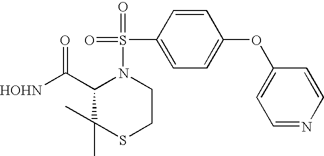 |
| Exemplary Non-hydroxamate MPI | | See David T. Puerta et al., “A Bioinorganic Perspective on Matrix Metalloproteinase Inhibition,” Current Topics in Medicinal Chemistry, 4, 1551-1573 (2004) incorporated herein by reference in its entirety. | 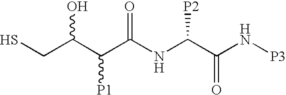 |

| P1 | P2 | P3 | Alcohol (nM) |
|---|---|---|---|
| i-butyl | t-butyl | methyl | <20000 |
| i-butyl | t-butyl | 2-pyridyl | 4600 |
| i-butyl | CHM | phenethyl | 1300 |
| n-heptyl | t-butyl | methyl | 120 |
| n-heptyl | t-butyl | PhSO₂NH₂ | 120 |
| n-heptyl | i-butyl | phenethyl | 1500 |
| n-heptyl | i-butyl | Morpholino | 5100 |
| n-heptyl | i-butyl | Leu(ethyl) | 210 |
| n-heptyl | CHM | PhSO₂NH₂ | 290 |
| phenpropyl | CHM | phenethyl | <2000 |

| | | |
|---|---|---|
| Exemplary Non-hydroxamate MPI | See David T. Puerta et al. supra. | 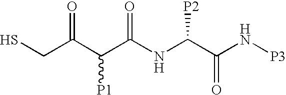 |

| P1 | P2 | P3 | Alcohol (nM) |
|---|---|---|---|
| i-butyl | t-butyl | methyl | 500 |
| i-butyl | t-butyl | 2-pyridyl | 160 |
| i-butyl | CHM | phenethyl | 98 |
| n-heptyl | t-butyl | methyl | 16 |
| n-heptyl | t-butyl | PhSO₂NH₂ | 22 |
| n-heptyl | i-butyl | phenethyl | 39 |
| n-heptyl | i-butyl | Morpholino | 130 |
| n-heptyl | i-butyl | Leu(ethyl) | 26 |
| n-heptyl | CHM | PhSO₂NH₂ | 43 |
| phenpropyl | CHM | phenethyl | 210 |

| | | |
|---|---|---|
| Batimastat | See David T. Puerta et al. supra. | 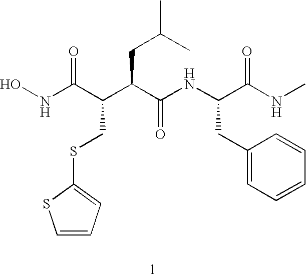 |
| WAY-170523 | See David T. Puerta et al. supra. | 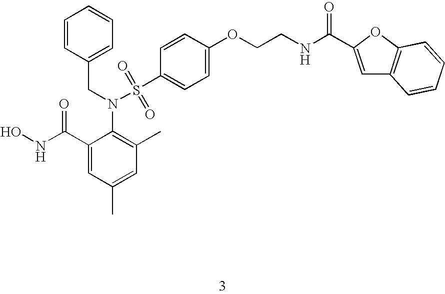 |
| (N-(2- hydroxamate- methylene-4-methyl- pentoyl)phenyl- alanyl)methylamine | See David T. Puerta et al. supra. | 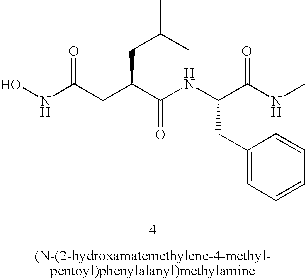 |
| 3-[4-[3- (cyanomethyl) phenyl]phenoxy] propano- hydroxamic acid | See David T. Puerta et al. supra. | 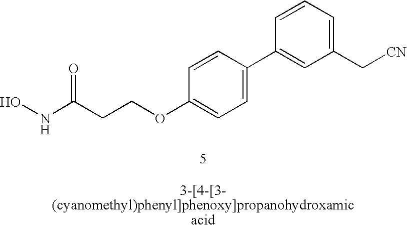 |
| The compounds of | Entitled SULFOXIMINE AND | |
| U.S. Pat. No. | SULDODIIMINE MATRIX | |
| 5,470,834, | METALLOPROTEINASE | |
| incorporated herein | INHIBITORS, issued to Schwartz et | |
| by reference | al., on Nov. 28, 1995 | |
| The compounds of | Entitled HYDROXAMIC ACID AND | |
| U.S. Pat. No. | CARBOXYLIC ACID | |
| 5,618,844, | DERIVATIVES, PROCESS FOR | |
| incorporated herein | THEIR PREPARATION AND USE | |
| by reference | THEREOF, issued to Gowravaram et | |
| | al., on April 8, 1997 | |
| The compounds of | Entitled α-AMINO SULFONYL | |
| U.S. Pat. No. | HYDROXAMIC ACIDS AS MATRIX | |
| 5,804,593, | METALLOPROTEINASE | |
| incorporated herein | INHIBITORS, issued to Warpehoski et | |
| by reference | al., on Sept., 8, 1998 | |
| The compounds of | Entitled MATRIX | |
| U.S. Pat. No. | METALLOPROTEINASE | |
| 5,917,090, | INHIBITORS, issued to Huxley et al., | |
| incorporated herein | on June 29, 1999 | |
| by reference | | |
| The compounds of | Entitled BUTYRIC ACID MATRIX | |
| U.S. Pat. No. | METALLOPROTEINASE | |
| 6,020,366, | INHIBITORS, issued to Picard et al., | |
| incorporated herein | on Feb. 1, 2000 | |
| by reference | | |
| The compounds of | Entitled MATRIX | |
| U.S. Pat. No. | METALLOPROTEINASE | |
| 6,194,451, | INHIBITORS, issued to Alpegiani et | |
| incorporated herein | al., on Feb. 27, 2001 | |
| by reference | | |
| The compounds of | Entitled MATRIX | |
| U.S. Pat. No. | METALLOPROTEINASE | |
| 6,277,876, | INHIBITORS, issued to Christensen, | |
| incorporated herein | on Aug., 21, 2001 | |
| by reference | | |
| The compounds of | Entitled DIBENZOFURAN | |
| U.S. Pat. No. | SULFONAMIDE MATRIX | |
| 6,294, 674, | METALLOPROTEINASE | |
| incorporated herein | INHIBITORS, issued to Picard et al., | |
| by reference | on Sept. 25, 2001 | |
| The compounds of | Entitled MATIX | |
| U.S. Pat. No. | METALLOPROTEINASE | |
| 6,294,694, | INHIBITORS AND METHOD OF | |
| incorporated herein | USING SAME, issued to Witiak et al., | |
| by reference | on Sept. 25, 2001 | |
| The compounds of | Entitled PREPARATION AND USE | |
| U.S. Pat. No. | OF ORTHO-SULFONAMIDO ARYL | |
| 6,465,508, | HYDROXAMIC ACIDS AS MATRIX | |
| incorporated herein | METALLOPROTEINASE | |
| by reference | INHIBITORS, issued to Nelson et al., | |
| | on Oct. 15, 2002 | |
| The compounds of | Entitled MATRIX | |
| U.S. Pat. No. | METALLOPROTEINASE | |
| 6,482,827, | INHIBITORS, issued to Alpegiani, et | |
| incorporated herein | al., on Nov. 19, 2002 | |
| by reference | | |
| The compounds of | Entitled MATRIX | |
| U.S. Pat. No. | METALLOPROTEINASE | |
| 6,521,606, | INHIBITORS, issued to Sorensen et | |
| incorporated herein | al., on Feb. 18, 2003 | |
| by reference | | |
| The compounds of | Entitled MATRIX | |
| U.S. Pat. No. | METALLOPROTEINASE | |
| 6,531,499, | INHIBITORS AND METHOD OF | |
| incorporated herein | USING SAME, issued to Witiak et al., | |
| by reference | on March 11, 2003 | |
| The compounds of | Entitled AROMATIC SULFONE | |
| U.S. Pat. No. | HYDROXAMIC ACID | |
| 6,541,489, | METALLOPROTEASE INHIBITOR, | |
| incorporated herein | issued to Barta et al., on April 1, 2003 | |
| by reference | | |
| The compounds of | Entitled α-AMINO-β-SULFONYL | |
| U.S. Pat. No. | HYDROXAMIC ACID | |
| 6,583,299, | COMPOUNDS, issued to Hockerman | |
| incorporated herein | et al., on June 24, 2003 | |
| by reference | | |
| The compounds of | Entitled MATRIX | |
| U.S. Pat. No. | METALLOPROTEINASE | |
| 6,600,057, | INHIBITORS, issued to Quirk, on | |
| incorporated herein | July 29, 2003 | |
| by reference | | |
| The compounds of | Entitled REMEDIES FOR JOINT | |
| U.S. Pat. No. | DISEASES, issued to Serizawa et al., | |
| 6,608,043, | on Aug. 19, 2003 | |
| incorporated herein | | |
| by reference | | |
| The compounds of | Entitled MATRIX | |
| U.S. Pat. No. | METALLOPROTEINASE | |
| 6,624,144, | INHIBITORS AND DOWN- | |
| incorporated herein | REGULATORS, issued to Koivunen et | |
| by reference | al., on Sept. 23, 2003 | |
| The compounds of | Entitled MATIX | |
| U.S. Pat. No. | METALLOPROTEINASE | |
| 6,624,177, | INHIBITORS AND THEIR | |
| incorporated herein | THERAPEUTIC USES, issued to | |
| by reference | O'Brien et al., on Sept. 23, 2003 | |
| The compounds of | Entitled MATRIX | |
| U.S. Pat. No. | METALLOPROTEINASE | |
| 6,656,448, | INHIBITORS, issued to Carpenter Jr. | |
| incorporated herein | et al., Dec. 2, 2003 | |
| by reference | | |
| The compounds of | Entitled PEPTIDE INHIBITOR OF | |
| U.S. Pat. No. | MMP ACTIVITY AND | |
| 6,667,388, | ANGIOGENESIS, issued to Bein et al., | |
| incorporated herein | on Dec. 23, 2003 | |
| by reference | | |
| The compounds of | Entitled HYDROXAMIC ACID | |
| U.S. Pat. No. | COMPOUNDS USEFUL AS | |
| 6,677,355, | MATRIX METALLOPROTEINASE | |
| incorporated herein | INHIBITORS, issued to Conrad et al., | |
| by reference | on Jan. 13, 2004 | |
| The compounds of | Entitled BIPHENYL | |
| U.S. Pat. No. | SULFONAMIDES USEFUL AS | |
| 6,686,355, | MATRIX METALLOPROTEINASE | |
| incorporated herein | INHIBITORS, issued to Barvian et al., | |
| by reference | on Feb. 3, 2004 | |
| The compounds of | Entitled AROMATIC SULFONE | |
| U.S. Pat. No. | HYDROXAMIC ACID | |
| 6,750,228, | METALLOPROTEASE INHIBITOR, | |
| incorporated herein | issued to Barta et al., on June 15, 2004 | |
| by reference | | |
| The compounds of | Entitled AROMATIC SULFONE | |
| U.S. Pat. No. | HYDROXAMIC ACID | |
| 6,750,233, | METALLOPROTEASE INHIBITOR, | |
| incorporated herein | issued to Barta et al., on June 15, 2004 | |
| by reference | | |
| The compounds of | Entitled 3-ARYLSULFONYL-2 | |
| U.S. Pat. No. | (SUBSTITUTED METHYL) | |
| 6765,003, | PROPANOIC ACID DERIVATIVES | |
| incorporated herein | AS MATRIX | |
| by reference | METALLOPROTEINASE | |
| | INHIBITORS, issued to Mantegani et | |
| | al., on July 20, 2004 | |
| The compounds of | Entitled PREPARATION AND USE | |
| U.S. Pat. No. | OF ORTHO-SULFONAMIDO | |
| 6,825,352, | ARYLHYDROXAMIC ACIDS AS | |
| incorporated herein | MATRIX METALLOPROTEINASE | |
| by reference | INHIBITORS, issued to Nelson et al., | |
| | on Nov. 30, 2004 | |
| The compounds of | Entitled AROMATIC SULFONE | |
| U.S. Pat. No. | HYDROXAMIC ACID | |
| 6,890,937, | METALLOPROTEASE INHIBITOR, | |
| incorporated herein | issued to Barta et al., on May 10, 2005 | |
| by reference | | |
| The compounds of | Entitled THIAZEPINYL | |
| U.S. Pat. No. | HYDROXAMIC ACID | |
| 6,967,197, | DERIVATIVES AS MATRIX | |
| incorporated herein | METALLOPROTEINASE | |
| by reference | INHIBITORS, issued to Neya et al., on | |
| | Nov. 22, 2005 | |
| The compounds of | Entitled MATRIX | |
| U.S. Pat. No. | METALLOPROTEINASE | |
| 6,989,139, | INHIBITORS, issued to Decicco et al., | |
| incorporated herein | on Jan. 24, 2006 | |
| by reference | | |
| The compounds of | Entitled MATRIX | |
| U.S. Pat. No. | METALLOPROTEINASE | |
| 7,060,248, | INHIBITORS, issued to Carpenter, Jr. | |
| incorporated herein | et al., on June 13, 2006 | |
| by reference | | |
| The compounds of | Entitled α-SULFONYLAMINO | |
| U.S. Pat. No. | HYDROXAMIC ACID INHIBITORS | |
| 6,417,229, | OF MATRIX | |
| incorporated herein | METALLOPROTEINASES FOR THE | |
| by reference | TREATMENT OF PERIPHERAL OR | |
| | CENTRAL NERVOUS SYSTEM | |
| | DISORDERS, issued to Sahagan et al., | |
| | on July 9, 2002 | |

### Methods of Treatment

The term “treating” refers to, and includes, reversing, alleviating, inhibiting the progress of, or preventing a disease, disorder or condition, or one or more symptoms thereof; and “treatment” and “therapeutically” refer to the act of treating, as defined herein.

A “therapeutically effective amount” is any amount of any of the compounds utilized in the course of practicing the invention provided herein that is sufficient to reverse, alleviate, inhibit the progress of, or prevent a disease, disorder or condition, or one or more symptoms thereof.

Certain embodiments herein relate to therapeutic compositions and methods of treatment for a subject by preventing or alleviating at least one symptom of inflammation associated with certain conditions or diseases. Many conditions or diseases associated with inflammation disorders have been treated with steroids, methotrexate, immunosuppressive drugs including cyclophosphamide, cyclosporine, azathioprine and leflunomide, nonsteroidal anti-inflammatory agents such as aspirin, acetaminophen and COX-2 inhibitors, gold agents and anti-malarial treatments.

### Routes and Forms of Administration

As used herein, “subject,” may refer to any living creature, preferably an animal, more preferably a mammal, and even more preferably a human.

In particular exemplary embodiments, the gas-enriched fluid of the present invention may function as a therapeutic composition alone or in combination with another therapeutic agent such that the therapeutic composition prevents or alleviates at least one symptom of inflammation. The therapeutic compositions of the present invention include compositions that are able to be administered to a subject in need thereof. In certain embodiments, the therapeutic composition formulation may also comprise at least one additional agent selected from the group consisting of: carriers, adjuvants, emulsifying agents, suspending agents, sweeteners, flavorings, perfumes, and binding agents.

As used herein, “pharmaceutically acceptable carrier” and “carrier” generally refer to a non-toxic, inert solid, semi-solid or liquid filler, diluent, encapsulating material or formulation auxiliary of any type. Some non-limiting examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. In particular aspects, such carriers and excipients may be gas-enriched fluids or solutions of the present invention.

The pharmaceutically acceptable carriers described herein, for example, vehicles, adjuvants, excipients, or diluents, are well known to those who are skilled in the art. Typically, the pharmaceutically acceptable carrier is chemically inert to the therapeutic agents and has no detrimental side effects or toxicity under the conditions of use. The pharmaceutically acceptable carriers can include polymers and polymer matrices, nanoparticles, microbubbles, and the like.

In addition to the therapeutic gas-enriched fluid of the present invention, the therapeutic composition may further comprise inert diluents such as additional non-gas-enriched water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. As is appreciated by those of ordinary skill, a novel and improved formulation of a particular therapeutic composition, a novel gas-enriched therapeutic fluid, and a novel method of delivering the novel gas-enriched therapeutic fluid may be obtained by replacing one or more inert diluents with a gas-enriched fluid of identical, similar, or different composition. For example, conventional water may be replaced or supplemented by a gas-enriched fluid produced by mixing oxygen into water or deionized water to provide gas-enriched fluid.

In certain embodiments, the inventive gas-enriched fluid may be combined with one or more therapeutic agents and/or used alone. In particular embodiments, incorporating the gas-enriched fluid may include replacing one or more solutions known in the art, such as deionized water, saline solution, and the like with one or more gas-enriched fluid, thereby providing an improved therapeutic composition for delivery to the subject.

Certain embodiments provide for therapeutic compositions comprising a gas-enriched fluid of the present invention, a pharmaceutical composition or other therapeutic agent or a pharmaceutically acceptable salt or solvate thereof, and at least one pharmaceutical carrier or diluent. These pharmaceutical compositions may be used in the prophylaxis and treatment of the foregoing diseases or conditions and in therapies as mentioned above. Preferably, the carrier must be pharmaceutically acceptable and must be compatible with, i.e. not have a deleterious effect upon, the other ingredients in the composition. The carrier may be a solid or liquid and is preferably formulated as a unit dose formulation, for example, a tablet that may contain from 0.05 to 95% by weight of the active ingredient.

Possible administration routes include oral, sublingual, buccal, parenteral (for example subcutaneous, intramuscular, intra-arterial, intraperitoneally, intracisternally, intravesically, intrathecally, or intravenous), rectal, topical including transdermal, intravaginal, intraoccular, intraotical, intranasal, inhalation, and injection or insertion of implantable devices or materials.

### Administration Routes

Most suitable means of administration for a particular subject will depend on the nature and severity of the disease or condition being treated or the nature of the therapy being used, as well as the nature of the therapeutic composition or additional therapeutic agent. In certain embodiments, oral or topical administration is preferred.

Formulations suitable for oral administration may be provided as discrete units, such as tablets, capsules, cachets, syrups, elixirs, chewing gum, “lollipop” formulations, microemulsions, solutions, suspensions, lozenges, or gel-coated ampules, each containing a predetermined amount of the active compound; as powders or granules; as solutions or suspensions in aqueous or non-aqueous liquids; or as oil-in-water or water-in-oil emulsions.

Formulations suitable for transmucosal methods, such as by sublingual or buccal administration include lozenges patches, tablets, and the like comprising the active compound and, typically a flavored base, such as sugar and acacia or tragacanth and pastilles comprising the active compound in an inert base, such as gelatin and glycerine or sucrose acacia.

Formulations suitable for parenteral administration typically comprise sterile aqueous solutions containing a predetermined concentration of the active gas-enriched fluid and possibly another therapeutic agent; the solution is preferably isotonic with the blood of the intended recipient. Additional formulations suitable for parenteral administration include formulations containing physiologically suitable co-solvents and/or complexing agents such as surfactants and cyclodextrins. Oil-in-water emulsions may also be suitable for formulations for parenteral administration of the gas-enriched fluid. Although such solutions are preferably administered intravenously, they may also be administered by subcutaneous or intramuscular injection.

Formulations suitable for urethral, rectal or vaginal administration include gels, creams, lotions, aqueous or oily suspensions, dispersible powders or granules, emulsions, dissolvable solid materials, douches, and the like. The formulations are preferably provided as unit-dose suppositories comprising the active ingredient in one or more solid carriers forming the suppository base, for example, cocoa butter. Alternatively, colonic washes with the gas-enriched fluids of the present invention may be formulated for colonic or rectal administration.

Formulations suitable for topical, intraoccular, intraotic, or intranasal application include ointments, creams, pastes, lotions, pastes, gels (such as hydrogels), sprays, dispersible powders and granules, emulsions, sprays or aerosols using flowing propellants (such as liposomal sprays, nasal drops, nasal sprays, and the like) and oils. Suitable carriers for such formulations include petroleum jelly, lanolin, polyethyleneglycols, alcohols, and combinations thereof. Nasal or intranasal delivery may include metered doses of any of these formulations or others. Likewise, intraotic or intraocular may include drops, ointments, irritation fluids and the like.

Formulations of the invention may be prepared by any suitable method, typically by uniformly and intimately admixing the gas-enriched fluid optionally with an active compound with liquids or finely divided solid carriers or both, in the required proportions and then, if necessary, shaping the resulting mixture into the desired shape.

For example a tablet may be prepared by compressing an intimate mixture comprising a powder or granules of the active ingredient and one or more optional ingredients, such as a binder, lubricant, inert diluent, or surface active dispersing agent, or by molding an intimate mixture of powdered active ingredient and a gas-enriched fluid of the present invention.

Suitable formulations for administration by inhalation include fine particle dusts or mists which may be generated by means of various types of metered dose pressurized aerosols, nebulisers, or insufflators. In particular, powders or other compounds of therapeutic agents may be dissolved or suspended in a gas-enriched fluid of the present invention.

For pulmonary administration via the mouth, the particle size of the powder or droplets is typically in the range 0.5-10 μM, preferably 1-5 μM, to ensure delivery into the bronchial tree. For nasal administration, a particle size in the range 10-500 μM is preferred to ensure retention in the nasal cavity.

Metered dose inhalers are pressurized aerosol dispensers, typically containing a suspension or solution formulation of a therapeutic agent in a liquefied propellant. In certain embodiments, as disclosed herein, the gas-enriched fluids of the present invention may be used in addition to or instead of the standard liquefied propellant. During use, these devices discharge the formulation through a valve adapted to deliver a metered volume, typically from 10 to 150 μL, to produce a fine particle spray containing the therapeutic agent and the gas-enriched fluid. Suitable propellants include certain chlorofluorocarbon compounds, for example, dichlorodifluoromethane, trichlorofluoromethane, dichlorotetrafluoroethane and mixtures thereof.

The formulation may additionally contain one or more co-solvents, for example, ethanol surfactants, such as oleic acid or sorbitan trioleate, anti-oxidants and suitable flavoring agents. Nebulisers are commercially available devices that transform solutions or suspensions of the active ingredient into a therapeutic aerosol mist either by means of acceleration of a compressed gas (typically air or oxygen) through a narrow venturi orifice, or by means of ultrasonic agitation. Suitable formulations for use in nebulisers consist of another therapeutic agent in a gas-enriched fluid and comprising up to 40% w/w of the formulation, preferably less than 20% w/w. In addition, other carriers may be utilized, such as distilled water, sterile water, or a dilute aqueous alcohol solution, preferably made isotonic with body fluids by the addition of salts, such as sodium chloride. Optional additives include preservatives, especially if the formulation is not prepared sterile, and may include methyl hydroxy-benzoate, anti-oxidants, flavoring agents, volatile oils, buffering agents and surfactants.

Suitable formulations for administration by insufflation include finely comminuted powders that may be delivered by means of an insufflator or taken into the nasal cavity in the manner of a snuff. In the insufflator, the powder is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the active ingredient or of a powder blend comprising the active ingredient, a suitable powder diluent, such as lactose, and an optional surfactant. The active ingredient typically comprises from 0.1 to 100 w/w of the formulation.

In addition to the ingredients specifically mentioned above, the formulations of the present invention may include other agents known to those skilled in the art, having regard for the type of formulation in issue. For example, formulations suitable for oral administration may include flavoring agents and formulations suitable for intranasal administration may include perfumes.

The therapeutic compositions of the invention can be administered by any conventional method available for use in conjunction with pharmaceutical drugs, either as individual therapeutic agents or in a combination of therapeutic agents.

The dosage administered will, of course, vary depending upon known factors, such as the pharmacodynamic characteristics of the particular agent and its mode and route of administration; the age, health and weight of the recipient; the nature and extent of the symptoms; the kind of concurrent treatment; the frequency of treatment; and the effect desired. A daily dosage of active ingredient can be expected to be about 0.001 to 1000 milligrams (mg) per kilogram (kg) of body weight, with the preferred dose being 0.1 to about 30 mg/kg.

Dosage forms (compositions suitable for administration) contain from about 1 mg to about 500 mg of active ingredient per unit. In these pharmaceutical compositions, the active ingredient will ordinarily be present in an amount of about 0.5-95% weight based on the total weight of the composition.

Ointments, pastes, foams, occlusions, creams and gels also can contain excipients, such as starch, tragacanth, cellulose derivatives, silicones, bentonites, silica acid, and talc, or mixtures thereof. Powders and sprays also can contain excipients such as lactose, talc, silica acid, aluminum hydroxide, and calcium silicates, or mixtures of these substances. Solutions of nanocrystalline antimicrobial metals can be converted into aerosols or sprays by any of the known means routinely used for making aerosol pharmaceuticals. In general, such methods comprise pressurizing or providing a means for pressurizing a container of the solution, usually with an inert carrier gas, and passing the pressurized gas through a small orifice. Sprays can additionally contain customary propellants, such as nitrogen, carbon dioxide, and other inert gases. In addition, microspheres or nanoparticles may be employed with the gas-enriched therapeutic compositions or fluids of the present invention in any of the routes required to administer the therapeutic compounds to a subject.

The injection-use formulations can be presented in unit-dose or multi-dose sealed containers, such as ampules and vials, and can be stored in a freeze-dried (lyophilized) condition requiring only the addition of the sterile liquid excipient, or gas-enriched fluid, immediately prior to use. Extemporaneous injection solutions and suspensions can be prepared from sterile powders, granules, and tablets. The requirements for effective pharmaceutical carriers for injectable compositions are well known to those of ordinary skill in the art. See, for example, Pharmaceutics and Pharmacy Practice, J. B. Lippincott Co., Philadelphia, Pa., Banker and Chalmers, Eds., 238-250 (1982) and ASHP Handbook on Injectable Drugs, Toissel, 4th ed., 622-630 (1986).

Formulations suitable for topical administration include lozenges comprising a gas-enriched fluid of the invention and optionally, an additional therapeutic and a flavor, usually sucrose and acacia or tragacanth; pastilles comprising a gas-enriched fluid and optional additional therapeutic agent in an inert base, such as gelatin and glycerin, or sucrose and acacia; and mouth washes or oral rinses comprising a gas-enriched fluid and optional additional therapeutic agent in a suitable liquid carrier; as well as creams, emulsions, gels and the like.

Additionally, formulations suitable for rectal administration may be presented as suppositories by mixing with a variety of bases such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

Suitable pharmaceutical carriers are described in Remington's Pharmaceutical Sciences, Mack Publishing Company, a standard reference text in this field.

The dose administered to a subject, especially an animal, particularly a human, in the context of the present invention should be sufficient to effect a therapeutic response in the animal over a reasonable time frame. One skilled in the art will recognize that dosage will depend upon a variety of factors including the condition of the animal, the body weight of the animal, as well as the condition being treated. A suitable dose is that which will result in a concentration of the therapeutic composition in a subject that is known to affect the desired response.

The size of the dose also will be determined by the route, timing and frequency of administration as well as the existence, nature, and extent of any adverse side effects that might accompany the administration of the therapeutic composition and the desired physiological effect.

It will be appreciated that the compounds of the combination may be administered: (1) simultaneously by combination of the compounds in a co-formulation or (2) by alternation, i.e. delivering the compounds serially, sequentially, in parallel or simultaneously in separate pharmaceutical formulations. In alternation therapy, the delay in administering the second, and optionally a third active ingredient, should not be such as to lose the benefit of a synergistic therapeutic effect of the combination of the active ingredients. According to certain embodiments by either method of administration (1) or (2), ideally the combination should be administered to achieve the most efficacious results. In certain embodiments by either method of administration (1) or (2), ideally the combination should be administered to achieve peak plasma concentrations of each of the active ingredients. A one pill once-per-day regimen by administration of a combination co-formulation may be feasible for some patients suffering from inflammatory neurodegenerative diseases. According to certain embodiments effective peak plasma concentrations of the active ingredients of the combination will be in the range of approximately 0.001 to 100 μM. Optimal peak plasma concentrations may be achieved by a formulation and dosing regimen prescribed for a particular patient. It will also be understood that the inventive fluids and a glucocorticoid steroid (e.g., Budesonide) or the physiologically functional derivatives of any thereof, whether presented simultaneously or sequentially, may be administered individually, in multiples, or in any combination thereof. In general, during alternation therapy (2), an effective dosage of each compound is administered serially, where in co-formulation therapy (1), effective dosages of two or more compounds are administered together.

The combinations of the invention may conveniently be presented as a pharmaceutical formulation in a unitary dosage form. A convenient unitary dosage formulation contains the active ingredients in any amount from 1 mg to 1 g each, for example but not limited to, 10 mg to 300 mg. The synergistic effects of the inventive fluid in combination with, for example, a glucocorticoid steroid (e.g., Budesonide) may be realized over a wide ratio, for example 1:50 to 50:1 (inventive fluid: a glucocorticoid steroid (e.g., Budesonide)). In one embodiment the ratio may range from about 1:10 to 10:1. In another embodiment, the weight/weight ratio of inventive fluid to a glucocorticoid steroid (e.g., Budesonide) in a co-formulated combination dosage form, such as a pill, tablet, caplet or capsule will be about 1, i.e. an approximately equal amount of inventive fluid and a glucocorticoid steroid (e.g., Budesonide). In other exemplary co-formulations, there may be more or less inventive fluid and a glucocorticoid steroid (e.g., Budesonide). In one embodiment, each compound will be employed in the combination in an amount at which it exhibits anti-inflammatory activity when used alone. Other ratios and amounts of the compounds of said combinations are contemplated within the scope of the invention.

A unitary dosage form may further comprise inventive fluid and, for example, a glucocorticoid steroid (e.g., Budesonide), or physiologically functional derivatives of either thereof, and a pharmaceutically acceptable carrier.

It will be appreciated by those skilled in the art that the amount of active ingredients in the combinations of the invention required for use in treatment will vary according to a variety of factors, including the nature of the condition being treated and the age and condition of the patient, and will ultimately be at the discretion of the attending physician or health care practitioner. The factors to be considered include the route of administration and nature of the formulation, the animal's body weight, age and general condition and the nature and severity of the disease to be treated.

It is also possible to combine any two of the active ingredients in a unitary dosage form for simultaneous or sequential administration with a third active ingredient. The three-part combination may be administered simultaneously or sequentially. When administered sequentially, the combination may be administered in two or three administrations. According to certain embodiments the three-part combination of inventive fluid and a glucocorticoid steroid (e.g., Budesonide) may be administered in any order.

According to particular aspects, the inventive electrokinetically-altered fluids, have substantial utility for treating MMP-9-mediated conditions, including but not limited to the exemplary genus of indications disclosed herein. According to additional aspects, the inventive electrokinetically-altered fluids, have utility for treating various subgenera of the exemplary genus, wherein at least one indication of the genus is excluded from each of said subgenera.

### Example 1

### Synergistic Effects of Inventive Electrokinetically-Altered Fluids and Albuterol were Demonstrated

Overview. The inventive electrokinetically-altered fluids provided for synergistic prolongation effects (e.g., suppression of bronchoconstriction) with Albuterol in vivo in an art-recognized animal model of human bronchoconstriction (human asthma model)) and thus provides for a decrease in a patient's albuterol usage. The results disclosed in this Example are also disclosed in Applicants' WO 2009/055729.

First experiment. In a first experiment, sixteen guinea pigs were evaluated for the effects of bronchodilators on airway function in conjunction with methacholine-induced bronchoconstriction. Following determination of optimal dosing, each animal was dosed with 50 μg/mL to deliver the target dose of 12.5 μg of albuterol sulfate in 250 μL per animal. The study was a randomized blocked design for weight and baseline PenH values. Two groups (A and B) received an intratracheal instillation of 250 μL of 50 μg/mL albuterol sulfate in one or two diluents: Group A was deionized water that had passed through the inventive device, without the addition of oxygen, while Group B was inventive gas-enriched water. Each group was dosed intratracheally with solutions using a Penn Century Microsprayer. In addition, the animals were stratified across BUXCO plethysmograph units so that each treatment group is represented equally within nebulizers feeding the plethysmographs and the recording units. Animals that displayed at least 75% of their baseline PenH value at 2 hours following albuterol administration were not included in the data analyses. This exclusion criteria is based on past studies where the failure to observe bronchoprotection with bronchodilators can be associated with dosing errors. As a result, one animal from the control group was dismissed from the data analyses. Once an animal had greater than 50% bronchoconstriction, the animal was considered to be not protected. The results indicate that 50% of the Group B animals were protected from bronchoconstriction out to 10 hours (at which time the test was terminated).

Second experiment. An additional set of experiments was conducted using a larger number of animals to evaluate the protective effects of the inventive electrokinetically generated fluids (e.g, RDC1676-00, RDC1676-01, RDC1676-02 and RDC1676-03) against methacholine-induced bronchoconstriction when administered alone or as diluents for albuterol sulfate in male guinea pigs.

Materials and methods. Guinea Pigs (Cavia porcellus) were Hartley albino, Crl:(HA)BR from Charles River Canada Inc. (St. Constant, Quebec, Canada). Weight: Approximately 325±50 g at the onset of treatment; number of groups was 32, with 7 male animals per group (plus 24 spares form same batch of animals). Diet; all animals had free access to a standard certified pelleted commercial laboratory diet (PMI Certified Guinea Pig 5026; PMI Nutrition International Inc.) except during designated procedures. Route of administration was intratracheal instillation via a Penn Century Microsprayer and methacholine challenge via whole body inhalation. The intratracheal route was selected to maximize lung exposure to the test article/control solution. Whole body inhalation challenge has been selected for methacholine challenge in order to provoke an upper airway hypersensitivity response (i.e. bronchoconstriction). Duration of treatment was one day.

Experimental design. All animals were subjected to inhalation exposure of methacholine (500 μg/ml), 2 hours following TA/Control administration. All animals received a dose volume of 250 μl. Therefore, albuterol sulfate was diluted (in the control article and the 4 test articles) to concentrations of 0, 25, 50 and 100 μg/ml. Thirty minutes prior to dosing, solutions of albuterol sulfate of 4 different concentrations (0, 25, 50 and 100 μg/ml) was made up in a 1 Ox stock (500 μg/mL) in each of these four test article solutions (RDC1676-00, RDC1676-01, RDC1676-02; and RDC1676-03). These concentrations of albuterol sulfate were also made up in non-electrokinetically generated control fluid (control 1). The dosing solutions were prepared by making the appropriate dilution of each stock solution. All stock and dosing solutions were maintained on ice once prepared. The dosing was completed within one hour after the test/control articles are made. A solution of methacholine (500 μg/ml) was prepared on the day of dosing.

Each animal received an intratracheal instillation of test or control article using a Penn Century microsprayer. Animals were food deprived overnight and were anesthetized using isoflurane, the larynx was visualized with the aid of a laryngoscope (or suitable alternative) and the tip of the microsprayer was inserted into the trachea. A dose volume of 250 μl/animal of test article or control was administered. The methacholine aerosol was generated into the air inlet of a mixing chamber using aeroneb ultrasonic nebulizers supplied with air from a Buxco bias flow pump. This mixing chamber in turn fed four individual whole body unrestrained plethysmographs, each operated under a slight negative pressure maintained by means of a gate valve located in the exhaust line. A vacuum pump was used to exhaust the inhalation chamber at the required flow rate.

Prior to the commencement of the main phase of the study, 12 spare animals were assigned to 3 groups (n=4/group) to determine the maximum exposure period at which animals may be exposed to methacholine to induce a severe but non-fatal acute bronchoconstriction. Four animals were exposed to methacholine (500 μg/mL) for 30 seconds and respiratory parameters were measured for up to 10 minutes following commencement of aerosol. Methacholine nebulizer concentration and/or exposure time of aerosolization was adjusted appropriately to induce a severe but non-fatal acute/reversible bronchoconstriction, as characterized by an transient increase in penes.

Once prior to test article administration (Day −1) and again at 2, 6, 10, 14, 18, 22 and 26 hours post-dose, animals were placed in the chamber and ventilatory parameters (tidal volume, respiratory rate, derived minute volume) and the enhanced pause Penh were measured for a period of 10 minutes using the Buxco Electronics BioSystem XA system, following commencement of aerosol challenge to methacholine. Once animals were within chambers baseline, values were recorded for 1-minute, following which methacholine, nebulizer concentration of 500 ug/mL were aerosoloized for 30 seconds, animals were exposed to the aerosol for further 10 minutes during which time ventilatory parameters were continuously assessed. Penh was used as the indicator of bronchoconstriction; Penh is a derived value obtained from peak inspiratory flow, peak expiratory flow and time of expiration. Penh=(Peak expiratory flow/Peak inspiratory flow)*(Expiratory time/time to expire 65% of expiratory volume−1).

Animals that did not display a severe acute broncoconstriction during the predose methacholine challenge were replaced. Any animal displaying at least 75% of their baseline PenhPenes value at 2 hours post dose were not included in the data analysis. The respiratory parameters were recorded as 20 second means. Data considered unphysiological was excluded from further analysis. Changes in Penh were plotted over a 15 minute period and Penh value was expressed as area under the curve. Numerical data was subjected to calculation of group mean values and standard deviations (as applicable).

Results. The results from this experiment showed that in the absence of Albuterol, administration of the inventive electrokinetically generated fluids had no apparent effect on mean percent baseline PenH values, when measured over a 26 hour period. Surprisingly, however, administration of albuterol (representative data for the 25 μg albuterol/animal groups are shown) formulated in the inventive electrokinetically generated fluids (at all oxygen level values tested; ambient, 20 ppm, 40 ppm and 60 ppm) resulted in a striking prolongation of anti-broncoconstrictive effects of albuterol, compared to control fluid. That is, the methacholine results showed a prolongation of the bronchodilation of albuterol out to at least 26 hours. Applicants also showed that there were consistent differences at all oxygen levels between RDC1676 and the normal saline control. Combining all 4 RDC1676 fluids, the p value for the overall treatment difference from normal saline was 0.03.

According to particular aspects, therefore, the inventive electrokinetically generated solutions provide for synergistic prolongation effects with Albuterol, thus providing for a decrease in a patient's albuterol usage, enabling more efficient cost-effective drug use, fewer side effects, and increasing the period over which a patient may be treated and responsive to treatment with albuterol.

### Example 2

### Effects of Inventive Electrokinetically-Altered Fluids on Cytokine Expression were Determined

Overview. The inventive electrokinetically-altered fluids lowered the production of pro-inflammatory cytokines (IL-1β, TNF-α, IL-6, and GM-CSF), chemokines (IL-8, MIP-1α, RANTES, and Eotaxin), inflammatory enzymes (iNOS, COX-2, and MMP-9), allergen responses (MHC class II, CD23, B7-1, and B7-2), and Th2 cytokines (IL-4, IL-13, and IL-5) when compared to control fluid and increased anti-inflammatory cytokines (e.g., IL1R-α, TIMPs) when compared to control fluid. The results disclosed in this Example are also disclosed in Applicants' WO 2009/055729.

In particular aspects, human mixed lymphocytes were stimulated with T3 antigen or PHA in Revalesio oxygen-enriched fluid, or control fluid, and changes in IL-1β, IL-2, IL-4, IL-5, IL-6, IL-7, IL-8, IL-10, IL-12(p40), IL-12(p70), IL-13, IL-17, Eotaxin, IFN-γ, GM-CSF, MIP-1β, MCP-1, G-CSF, FGFb, VEGF, TNF-α, RANTES, Leptin, TNF-β, TFG-β, and NGF were evaluated. As was shown, pro-inflammatory cytokines (IL-1β, TNF-α, IL-6, and GM-CSF), chemokines (IL-8, MIP-1α, RANTES, and Eotaxin), inflammatory enzymes (iNOS, COX-2, and MMP-9), allergen responses (MHC class II, CD23, B7-1, and B7-2), and Th2 cytokines (IL-4, IL-13, and IL-5) tested were reduced in test fluid versus control fluid. By contrast, anti-inflammatory cytokines (e.g., IL1R-α, TIMPs) tested were increased in test fluid versus control fluid.

Additionally, Applicants used an art recognized model system involving ovalbumin sensitization, for assessing allergic hypersensitivity reactions. The end points studied were particular cytologic and cellular components of the reaction as well as serologic measurements of protein and LDH. Cytokine analysis was performed, including analysis of Eotaxin, IL-1A, IL-1B, KC, MCP-1, MCP-3, MIP-1A, RANTES, TNF-A, and VCAM.

Briefly, male Brown Norway rats were injected intraperitoneally with 0.5 mL Ovalbumin (OVA) Grade V (A5503-1G, Sigma) in solution (2.0 mg/mL) containing aluminum hydroxide (Al(OH)₃) (200 mg/mL) once each on days 1, 2, and 3. The study was a randomized 2×2 factorial arrangement of treatments (4 groups). After a two week waiting period to allow for an immune reaction to occur, the rats were either exposed or were treated for a week with either RDC1676-00 (sterile saline processed through the Revalesio proprietary device), and RDC1676-01 (sterile saline processed through the Revalesio proprietary device with additional oxygen added). At the end of the 1 week of treatment for once a day, the 2 groups were broken in half and 50% of the rats in each group received either Saline or OVA challenge by inhalation.

Specifically, fourteen days following the initial serialization, 12 rats were exposed to RDC 1676-00 by inhalation for 30 minutes each day for 7 consecutive days. The air flow rate through the system was set at 10 liters/minute. A total of 12 rats were aligned in the pie chamber, with a single port for nebulized material to enter and evenly distribute to the 12 sub-chambers of the Aeroneb.

Fifteen days following initial sensitization, 12 rats were exposed to RDC 1676-01 by ultrasonic nebulization for 30 minutes each day for 7 consecutive days. The air flow was also set for 10 liters/minute, using the same nebulizer and chamber. The RDC 1676-00 was nebulized first and the Aeroneb chamber thoroughly dried before RDC 1676-01 was nebulized.

Approximately 2 hours after the last nebulization treatment, 6 rats from the RDC 1676-00 group were re-challenged with OVA (1% in saline) delivered by intratreacheal instillation using a Penn Century Microsprayer (Model 1A-1B). The other 6 rats from the RDC 1676-00 group were challenged with saline as the control group delivered by way of intratreacheal instillation. The following day, the procedure was repeated with the RDC 1676-01 group.

Twenty four hours after re-challenge, all rats in each group were euthanized by overdose with sodium pentobarbital. Whole blood samples were collected from the inferior vena-cava and placed into two disparate blood collection tubes: Qiagen PAXgene™ Blood RNA Tube and Qiagen PAXgene™ Blood DNA Tube. Lung organs were processed to obtain bronchoalveolar lavage (BAL) fluid and lung tissue for RT-PCR to assess changes in markers of cytokine expression known to be associated with lung inflammation in this model. A unilateral lavage technique was be employed in order to preserve the integrity of the 4 lobes on the right side of the lung. The left “large” lobe was lavaged, while the 4 right lobes were tied off and immediately placedinot TRI-Zol™, homogenized, and sent to the lab for further processing.

BAL analysis. Lung lavage was collected and centrifuged for 10 minutes at 4° C. at 600-800 g to pellet the cells. The supernatants were transferred to fresh tubes and frozen at −80° C. Bronchial lavage fluid (“BAL”) was separated into two aliquots. The first aliquot was spun down, and the supernatant was snap frozen on crushed dry ice, placed in −80° C., and shipped to the laboratory for further processing. The amount of protein and LDH present indicates the level of blood serum protein (the protein is a serum component that leaks through the membranes when it's challenged as in this experiment) and cell death, respectively. The proprietary test side showed slight less protein than the control.

The second aliquot of bronchial lavage fluid was evaluated for total protein and LDH content, as well as subjected to cytological examination. The treated group showed total cells to be greater than the saline control group. Further, there was an increase in eosinophils in the treated group versus the control group. There were also slightly different polymorphonuclear cells for the treated versus the control side.

Blood analysis. Whole blood was analyzed by transfer of 1.2-2.0 mL blood into a tube, and allowing it to clot for at least 30 minutes. The remaining blood sample (approximately 3.5-5.0 mL) was saved for RNA extraction using TRI-Zol™ or PAXgene™. Next, the clotted blood sample was centrifuged for 10 minutes at 1200 g at room temperature. The serum (supernatant) was removed and placed into two fresh tubes, and the serum was stored at −80° C.

For RNA extraction utilizing Tri-Reagent (TB-126, Molecular Research Center, Inc.), 0.2 mL of whole blood or plasma was added to 0.75 mL of TRI Reagent BD supplemented with 20 μL of 5N acetic acid per 0.2 mL of whole blood or plasma. Tubes were shaken and stored at −80° C. Utilizing PAXgene™, tubes were incubated for approximately two hours at room temperature. Tubes were then placed on their side and stored in the −20° C. freezer for 24 hours, and then transferred to −80° C. for long term storage.

Luminex analysis. By Luminex platform, a microbead analysis was utilized as a substrate for an antibody-related binding reaction which is read out in luminosity units and can be compared with quantified standards. Each blood sample was run as 2 samples concurrently. The units of measurement are luminosity units and the groups are divided up into OVA challenged controls, OVA challenged treatment, and saline challenged treatment with proprietary fluid.

For Agilant gene array data generation, lung tissue was isolated and submerged in TRI Reagent (TR118, Molecular Research Center, Inc.). Briefly, approximately 1 mL of TRI Reagent was added to 50-100 mg of tissue in each tube. The samples were homogenized in TRI Reagent, using glass-Teflon™ or Polytron™ homogenizer. Samples were stored at −80° C.

Results from Blood Samples. Each blood sample was split into 2 samples and the samples were run concurrently. The units of measure are units of luminosity and the groups, going from left to right are: OVA challenged controls; OVA challenged Revalesio treatment; followed by saline challenged saline treatment; and saline challenged Revalesio treatment. To facilitate review, both the RDC1676-01 groups are highlighted with gray shaded backdrops, whereas the control saline treatment groups have unshaded backdrops.

Generally, in comparing the two left groups, while the spread of the RDC1676-01 group data is somewhat greater, particular cytokine levels in the RDC 1676-01 group as a whole are less than the samples in the control treated group; typically about a 30% numerical difference between the 2 groups. Generally, in comparing the right-most two groups, the RDC1676-01 group has a slightly higher numerical number compared to the RDC1676-00 group.

Applicants determined that the level of RANTES (IL-8 super family) produced after treatment with the inventive electrokinetically-altered fluids was less than that produced by the saline only exposed groups. Applicants demonstrated that the inventive electrokinetically-altered fluids caused MCP-1 to be produce at lower levels when compared to that which was produced by the saline only exposed groups. Applicants determined that the level of TNF alpha produced after treatment with the inventive electrokinetically-altered fluids was less than that produced by the saline only exposed groups.

In addition, Applicants demonstrated that the level of MIP-1 alpha produced after treatment with the inventive electrokinetically-altered fluids was less than that produced by the saline only exposed groups. Applicants demonstrated that the inventive electrokinetically-altered fluids caused IL-1 alpha to be produce at lower levels when compared to that which was produced by the saline only exposed groups. Applicants observed that the level of Vcam produced after treatment with the inventive electrokinetically-altered fluids was less than that produced by the saline only exposed groups. Applicants observed that the level of IL-1 beta produced after treatment with the inventive electrokinetically-altered fluids was less than that produced by the saline only exposed groups. Applicants demonstrated that the inventive electrokinetically-altered fluids caused Eotaxin and MCP-3 to be produce at lower levels when compared to that which was produced by the saline only exposed groups.

In summary, this standard assay of inflammatory reaction to a known sensitization produced, at least in the blood samples, a marked clinical and serologic affect. Additionally, while significant numbers of control animals were physiologically stressed and nearly dying in the process, none of the RDC1676-01 treated group showed such clinical stress effects. This was reflected then in the circulating levels of cytokines, with approximately 30% differences between the RDC1676-01-treated and the RDC1676-01-treated groups in the OVA challenged groups. By contrast, there were small and fairly insignificant changes in cytokine, cellular and serologic profiles between the RDC1676-01-treated and the RDC1676-01-treated groups in the non-OVA challenged groups, which likely merely represent minimal baseline changes of the fluid itself.

### Example 3

### Effects of the Inventive Electrokinetically-Altered Fluids to Modulate T-Cell Proliferation were Determined

Overview. The inventive electrokinetically-altered fluids improved regulatory T-cell function as shown by relatively decreased proliferation. The results disclosed in this Example are also disclosed in Applicants' WO 2009/055729.

The ability of particular embodiments disclosed herein to regulate T cells was studied by irradiating antigen presenting cells, and introducing antigen and T cells. Typically, these stimulated T cells proliferate. However, upon the introduction of regulatory T cells, the usual T cell proliferation is suppressed.

Methods. Briefly, FITC-conjugated anti-CD25 (ACT-1) antibody used in sorting was purchased from DakoCytomation (Chicago, Ill.). The other antibodies used were as follows: CD3 (HIT3a for soluble conditions), GITR (PE conjugated), CD4 (Cy-5 and FITC-conjugated), CD25 (APC-conjugated), CD28 (CD28.2 clone), CD127-APC, Granzyme A (PE-conjugated), FoxP3 (BioLegend), Mouse IgG1 (isotype control), and XCL1 antibodies. All antibodies were used according to manufacturer's instructions.

CD4+ T cells were isolated from peripheral whole blood with CD4+ Rosette Kit (Stemcell Technologies). CD4+ T cells were incubated with anti-CD127-APC, anti-CD25-PE and anti-CD4-FITC antibodies. Cells were sorted by flow cytometry using a FACS Aria into CD4+CD25hiCD127lo/nTreg and CD4+CD25-responder T cells.

Suppression assays were performed in round-bottom 96 well microtiter plates. 3.75×103 CD4+CD25neg responder T cells, 3.75×103 autologous T reg, 3.75×104 allogeneic irradiated CD3-depleted PBMC were added as indicated. All wells were supplemented with anti-CD3 (clone HIT3a at 5.0 ug/ml). T cells were cultured for 7 days at 37° C. in RPMI 1640 medium supplemented with 10% fetal bovine serum. Sixteen hours before the end of the incubation, 1.0 mCi of ³H-thymidine was added to each well. Plates were harvested using a Tomtec cell harvester and ³H-thymidine incorporation determined using a Perkin Elmer scintillation counter. Antigen-presenting cells (APC) consisted of peripheral blood mononuclear cells (PBMC) depleted of T cells using StemSep human CD3+ T cell depletion (StemCell Technologies) followed by 40 Gy of irradiation.

Regulatory T cells were stimulated with anti-CD3 and anti-CD28 conditions and then stained with Live/Dead Red viability dye (Invitrogen), and surface markers CD4, CD25, and CD127. Cells were fixed in the Lyze/Fix PhosFlow™ buffer and permeabilized in denaturing Permbuffer III®. Cells were then stained with antibodies against each particular selected molecule.

Statistical analysis was performed using the GraphPad Prism software. Comparisons between two groups were made by using the two-tailed, unpaired Student's t-test. Comparisons between three groups were made by using 1-way ANOVA. P values less than 0.05 were considered significant (two-tailed). Correlation between two groups were determined to be statistically significant via the Spearman coefficient if the r value was greater than 0.7 or less than −0.7 (two-tailed).

Results. Regulatory T cell proliferation was studied by stimulating cells with diesel exhaust particulate matter (PM, from EPA). Applicants determined that the cells stimulated with PM (no Rev, no Solas) resulted in a decrease in secreted IL-10, while cells exposed to PM in the presence of the fluids of the instant disclosure (“PM+Rev”) resulted in a maintained or only slightly decreased production of IL-10 relative to the Saline and Media controls (no PM). Furthermore, Diphtheria toxin (DT390, a truncated diphtheria toxin molecule; 1:50 dilution of std. commercial concentration) was titrated into inventive fluid samples, and blocked the Rev-mediated effect of increase in IL-10. Note that treatment with Rev alone resulted in higher IL-10 levels relative to Saline and Media controls. Similar results were obtained with GITR, Granzyme A, XCL1, pStat5, and Foxp3, respectively.

Applicants also obtained AA PBMC data, obtained from an allergic asthma (AA) profile of peripheral blood mononuclear cells (PBMC) evaluating tryptase. The AA PBMC data was consistent with the above T-regulatory cell data, as cells stimulated with particulate matter (PM) showed high levels of tryptase, while cells treated with PM in the presence of the fluids of the instant disclosure (“PM+Rev”) resulted in significantly lower tryptase levels similar to those of the Saline and Media controls. Consistent with the data from T-regulatory cells, exposure to DT390 blocked the Rev-mediated effect on tryptase levels, resulting in an elevated level of tryptase in the cells as was seen for PM alone (minus Rev, no Rev, no Solas). Treatment with Rev alone resulted in lower tryptase levels relative to Saline and Media controls.

In summary, Applicants observed a decreased proliferation in the presence of PM and Rev relative to PM in control fluid (no Rev, no Solas), indicating that the inventive electrokinetically generated fluid Rev improved regulatory T-cell function as shown by relatively decreased proliferation in the assay. Moreover, the evidence indicates that beta blockade, GPCR blockade and Ca channel blockade affects the activity of Rev on Treg function.

### Example 4

### Synergistic Effects Between the Inventive Electrokinetically-Altered Fluids and Budesonide were Determined

Overview. The inventive electrokinetically-altered fluids provided for synergistic anti-inflammatory effects with Budesonide in vivo in an art-recognized animal model for allergic asthma. The results disclosed in this Example are also disclosed in Applicants' WO 2009/055729.

Applicants initially performed experiments to assess the airway anti-inflammatory properties of the inventive electrokinetically generated fluids (e.g., RDC-1676-03) in a Brown Norway rat ovalbumin sensitization model. The Brown Norway rat is an art-recognized model for determining the effects of a test material on airway function and this strain has been widely used, for example, as a model of allergic asthma. Airway pathology and biochemical changes induced by ovalbumin sensitization in this model resemble those observed in man (Elwood et al., J Allergy Clin Immuno 88:951-60, 1991; Sirois & Bissonnette, Clin Exp Immunol 126:9-15, 2001). The inhaled route was selected to maximize lung exposure to the test material or the control solution. The ovalbumin-sensitized animals were treated with budesonide alone or in combination with the test material RDC 1676-03 for 7 days prior to ovalbumin challenge. 6 and 24 hours following the challenge, total blood count and levels of several pro and anti-inflammatory cytokines as well as various respiratory parameters were measured to estimate any beneficial effect of administering the test material on various inflammatory parameters.

### Materials and Methods:

Brown Norway rats of strain Bn/Crl were obtained from Charles River Kingston, weighing approximately 275±50 g at the onset of the experiment. All animal studies were conducted with the approval by PCS-MTL Institutional Animal Care and Use Committee. During the study, the use and care of animals were conducted according to guidelines of the USA National Research Council as well as Canadian Council of Animal Care.

Sensitization. On day 1 of the experiment, animals (14 animals in each treatment group) were sensitized by administration of a 1 ml intraperitoneal injection of a freshly prepared solution of 2 mg ovalbumin/100 mg Aluminum Hydroxide per 1 ml of 0.9% Sodium Chloride, followed by repeat injection on day 3.

Treatment. Fifteen days following the initial sensitization, animals were subjected to nebulized exposure to control (Normal saline) or test solutions (electrokinetically generated fluids RDC1676-00, RDC1676-02 and RDC-1676-03), either administered alone or in combination with Budesonide, once daily for 15 minutes for 7 consecutive days. Animals were dosed in a whole body chamber of approximately 20 L, and test atmosphere was generated into the chamber air inlet using aeroneb ultrasonic nebulizers supplied with air from a Buxco bias flow pump. The airflow rate was set at 10 liters/min.

Ovalbumin challenge. On day 21, 2 hours following treatment with the test solutions, all animals were challenged with 1% ovalbumin nebulized solution for 15 minutes (in a whole body chamber at airflow 2 L/min).

Sample collection. At time points of 6 and 24 hours after the ovalbumin challenge, blood samples were collected for total and differential blood cell counts as well as for measuring levels of various pro and anti-inflammatory cytokines. In addition, immediately after and at 6 and 24 hours following ovalbumin challenge the enhanced pause Penh and tidal volume were measured for a period of 10 minutes using the Buxco Electronics BioSystem XA system.

### Results:

Eosinophil Count: As expected, treatment with Budesonide (“NS+Budesonide 750 μg/Kg”; densely crosshatched bar graph) reduced the total eosinophil count in the challenged animals relative to treatment with the normal saline “NS” alone control. Additionally, while treatment with the inventive fluid “RDC1676-03” alone did not significantly reduce the eosinophil count, it nonetheless displayed a substantial synergy with Budesonide in reducing the eosinophil count (“RDC1676-03+Budesonide 750 μg/Kg). Similarly, the Eosinophil % also reflected a similar trend. While RDC1676-03 or Budesonide 750 ug/kg alone did not have a significant effect on Eosinophil % count in the challenged animals, the two in combination reduced the Eosinophil % significantly.

Therefore, Applicants determined, according to particular aspects, that the inventive electrokinetically generated fluids (e.g., RDC1676-03) have a substantial synergistic utility in combination with Budesonide to significantly reduce eosinophil count (“Eosinophil %” and total count) in an art-recognized rat model for human allergic asthma.

### Respiratory Parameters:

Applicants also demonstrated the observed effect of the test fluids on Penh and tidal volume as measured immediately, 6 and 24 hours after the ovalbumin challenge. Penh is a derived value obtained from peak inspiratory flow, peak expiratory flow and time of expiration and lowering of penh value reflects a favorable outcome for lung function.

Penh=(Peak expiratory flow/Peak inspiratory flow)*(Expiratory time/time to expire 65% of expiratory volume−1).

Treatment with Budesonide (at both 500 and 750 ug/kg) alone or in combination with any of the test fluids failed to significantly affect the Penh values immediately after the challenge. However, 6 hours after the challenge, animals treated with RDC 1676-03 alone or in combination with Budesonide 500 or 750 ug/kg demonstrated a significant drop in Penh values. Although the extent of this drop was diminished by 24 hours post challenge, the trend of a synergistic effect of Budesonide and RDC fluid was still observed at this time point.

Tidal volume is the volume of air drawn into the lungs during inspiration from the end-expiratory position, which leaves the lungs passively during expiration in the course of quiet breathing. Animals treated with Budesonide alone showed no change in tidal volumes immediately after the challenge. However, RDC1676-03 alone had a significant stimulatory effect on tidal volume even at this early time point. And again, RDC1676-03 in combination with Budesonide (both 500 and 750 ug/kg) had an even more pronounced effect on Tidal volume measurements at this time point. Six hours after the challenge, RDC1676-03 alone was sufficient to cause a significant increase in tidal volume and addition of Budesonide to the treatment regimen either alone or in combination had no added effect on tidal volume. Any effect observed at these earlier time points were, however, lost by the 24 hours time point.

Taken together, these data demonstrate that RDC1676-03 alone or in combination with Budesonide provided significant relief to airway inflammation as evidenced by increase in tidal volume and decrease in Penh values at 6 hours post challenge.

### Cytokine Analysis:

To analyze the mechanism of the effects seen on the above discussed physiological parameters, a number of pro as well as anti-inflammatory cytokines were measured in blood samples collected at 6 and 24 hours after the challenge, immediately following the physiological measurements.

Applicants observed that Rev 60 (or RDC1676-03) alone lowered the blood level of eotaxin significantly at both 6 and 24 hours post challenge. Budesonide 750 ug/kg also reduced the blood eotaxin levels at both of these time points, while Budesonide 250 ug/kg only had a notable effect at the later time point. However, the test solution Rev 60 alone showed effects that are significantly more potent (in reducing blood eotaxin levels) than both concentrations of Budesonide, at both time points. Eotaxin is a small C-C chemokine known to accumulate in and attract eosinophils to asthmatic lungs and other tissues in allergic reactions (e.g., gut in Crohn's disease). Eotaxin binds to a G protein coupled receptor CCR3. CCR3 is expressed by a number of cell types such as Th2 lymphocytes, basophils and mast cells but expression of this receptor by Th2 lymphocyte is of particular interest as these cells regulate eosinophil recruitment. Several studies have demonstrated increased production of eotaxin and CCR3 in asthmatic lung as well as establishing a link between these molecules and airway hyperresponsiveness (reviewed in Eotaxin and the attraction of eosinophils to the asthmatic lung, Dolores M Conroy and Timothy J Williams Respiratory Research 2001, 2:150-156).

Taken together these results strongly indicate that treatment with RDC1676-03 alone or in combination with Budesonide can significantly reduce eosinophil total count and % in blood 24 hours after the ovalbumin challenge. This correlates with a significant drop in eotaxin levels in blood observed as early as 6 hours post challenge.

Blood levels of two major key anti-inflammatory cytokines, IL10 and Interferon gamma are also significantly enhanced at 6 hours after challenge as a result of treatment with Rev 60 alone or in combination with Budesonide. Applicants observed such effects on Interferon gamma and IL 10, respectively. Rev 60 alone or Rev 60 in combination with Budesonide 250 ug/kg significantly increased the blood level of IL10 in the challenged animals up to 6 hrs post challenge. Similarly, Rev 60 alone or in combination with Budesonide 250 ug/kg or 750 ug/kg significantly increased the blood level of IFN gamma at 6 hours post challenge. Increase in these anti-inflammatory cytokines may well explain, at least in part, the beneficial effects seen on physiological respiratory parameters seen 6 hours post challenge. The effect on these cytokines was no longer observed at 24 hour post challenge (data not shown).

Rantes or CCL5 is a cytokine expressed by circulating T cells and is chemotactic for T cells, eosinophils and basophils and has an active role in recruiting leukocytes into inflammatory sites. Rantes also activates eosinophils to release, for example, eosinophilic cationic protein. It changes the density of eosinophils and makes them hypodense, which is thought to represent a state of generalized cell activation. It also is a potent activator of oxidative metabolism specific for eosinophils.

Applicants observed that systemic levels of Rantes was reduced significantly at 6 hours, but not at 24 hours post challenge in animals treated with Rev 60 alone or in combination of Budesonide 250 ug/kg or 750 ug/kg. Once again, there was a clear synergistic effect of Budesonide 750 ug/kg and Rev 60. A similar downward trend was observed for a number of other pro-inflammatory cytokines, such as KC or IL8, MCP3, IL1b, GCSF, TGFb as well as NGF, observed either at 6 or at 24 hours post challenge, in animals treated with Rev60 alone or in combination with Budesonide.

### Example 5

### Effects of the Inventive Electrokinetically-Altered Fluids on Intercellular Tight Junctions were Determined

Overview. The inventive electrokinetically-altered fluids were shown to modulate intercellular tight junctions. The results disclosed in this Example are also disclosed in Applicants' WO 2009/055729.

According to particular aspects, the inventive diffuser processed therapeutic fluids have substantial utility for modulating intercellular tight junctions, including those relating with pulmonary and systemic delivery and bioavailability of polypeptides, including the exemplary polypeptide salmon calcitonin (sCT).

Example Overview. Salmon calcitonin (sCT) is a 32 amino acid peptide with a molecular weight of 3,432 Daltons. Pulmonary delivery of calcitonin has been extensively studied in model systems (e.g., rodent model systems, rat model systems, etc) to investigate methods to enhance pulmonary drug delivery (e.g., intratracheal drug delivery). According to particular exemplary aspects, the inventive diffuser processed therapeutic fluid has substantial utility for modulating (e.g., enhancing) intercellular tight junctions, for example those associated with pulmonary and systemic delivery and bioavailability of sCT in a rat model system.

### Methods:

Intratracheal drug delivery. According to particular embodiments, sCT is formulated in the inventive therapeutic fluid and administered to rats using an intratracheal drug delivery device. In certain aspects, a Penn Century Micro-Sprayer device designed for rodent intratracheal drug delivery is used, allowing for good lung delivery, but, as appreciated in the art, with relatively low alveolar deposition resulting in poor systemic bioavailability of peptides. According to particular aspects, this art-recognized model system was used to confirm that the inventive diffuser processed therapeutic fluid has substantial utility for modulating (e.g., enhancing) intercellular tight junctions, including those associated with pulmonary and systemic delivery and bioavailability of polypeptides.

Animal groups and dosing. In certain aspects, rats are assigned to one of 3 groups (n=6 per group): a) sterile saline; b) base solution without O₂ enrichment (‘base solution’); or c) inventive diffuser processed therapeutic fluid (‘inventive enriched based solution’). The inventive enriched based solution is formed, for example by infusing oxygen in 0.9% saline. Preferably, the base solution comprises about 0.9% saline to minimize the potential for hypo-osmotic disruption of epithelial cells. In certain embodiments, sCT is separately reconstituted in the base solution and the inventive enriched based solution and the respective solutions are delivered to respective animal groups by intratracheal instillation within 60 minutes (10 μg sCT in 200 μL per animal).

Assays. In particular aspects, blood samples (e.g., 200 μl) are collected and placed into EDTA coated tubes prior to dosing and at 5, 10, 20, 30, 60, 120 and 240 minutes following dosing. Plasma is harvested and stored at ≦−70° C. until assayed for sCT using an ELISA.

For Agilant gene array data generation, lung tissue was isolated and submerged in TRI Reagent (TR118, Molecular Research Center, Inc.). Briefly, approximately 1 mL of TRI Reagent was added to 50-100 mg of tissue in each tube. The samples were homogenized in TRI Reagent, using glass-Teflon™ or Polytron homogenizer. Samples were stored at −80° C.

### Results:

Enhancement of tight junctions. Applicants observed that RDC1676-01 (sterile saline processed through the instant proprietary device with additional oxygen added; gas-enriched electrokinetically generated fluid (Rev) of the instant disclosure, decreased systemic delivery and bioavailability of sCT. According to particular aspects, the decreased systemic delivery results from decreased adsorption of sCT, most likely resulting from enhancement of pulmonary tight junctions. RDC1676-00 signifies sterile saline processed according to the presently disclosed methods, but without oxygenation.

Additionally, according to particular aspects, tight junction related proteins were upregulated in lung tissue. Applicants showed upregulation of the junction adhesion molecules JAM 2 and 3, GJA1, 3, 4 and 5 (junctional adherins), OCLN (occludin), claudins (e.g., CLDN 3, 5, 7, 8, 9, 10), TJP1 (tight junction protein 1), respectively.

### Example 6

### Effects of the Inventive Electrokinetically-Altered Fluids on Whole-Cell Conductance were Determined

Overview. The inventive electrokinetically-altered fluids decreased the whole-cell conductance as demonstrated by patch clamp analysis conducted on bronchial epithilial cells (BEC). Patch clamp analysis conducted on bronchial epithilial cells (BEC) perfused with inventive electrokinetically-altered fluid (RNS-60) revealed that exposure to RNS-60 resulted in a decrease in whole cell conductance. In addition, stimulation with a cAMP stimulating “cocktail”, which dramatically increased the whole-cell conductance, also increased the drug-sensitive portion of the whole-cell conductance, which was ten-times higher than that observed under basal conditions. The results disclosed in this Example are also disclosed in Applicants' WO 2009/055729.

Patch clamp studies were performed to further confirm the utility of the inventive electrokinetically generated fluids to modulate intracellular signal transduction by modulation of at least one of membrane structure, membrane potential or membrane conductivity, membrane proteins or receptors, ion channels, and calcium dependant cellular messaging systems.

Overview. Applicants showed that Bradykinin binding to the B2 receptor was concentration dependent, and binding affinity was increased in the electrokinetically generated fluid (e.g., Rev; gas-enriched electrokinetically generated fluid) of the instant disclosure compared to normal saline. Additionally, Applicants showed in the context of T-regulatory cells stimulated with particulate matter (PM), that there was a decreased proliferation of T-regulatory cells in the presence of PM and Rev relative to PM in control fluid (no Rev, no Solas), indicating that the inventive electrokinetically generated fluid Rev improved regulatory T-cell function; e.g., as shown by relatively decreased proliferation in the assay. Moreover, exposure to the inventive fluids resulted in a maintained or only slightly decreased production of IL-10 relative to the Saline and Media controls (no PM). Likewise, in the context of the allergic asthma (AA) profiles of peripheral blood mononuclear cells (PBMC) stimulated with particulate matter (PM), the data showed that exposure to the fluids of the instant disclosure (“PM+Rev”) resulted in significantly lower tryptase levels similar to those of the Saline and Media controls. Additionally, the Diphtheria toxin (DT390) effects indicate that beta blockade, GPCR blockade and Ca channel blockade affects the activity of the electrokinetically generated fluids on Treg and PBMC function. Furthermore, Applicants demonstrated, according to additional aspects, upon expose to the inventive fluids, tight junction related proteins were upregulated in lung tissue. Applicants showed upregulation of the junction adhesion molecules JAM 2 and 3, GJA1, 3, 4 and 5 (junctional adherins), OCLN (occludin), claudins (e.g., CLDN 3, 5, 7, 8, 9, 10), TJP1 (tight junction protein 1), respectively. Patch clamp studies were performed to further investigate and confirm said utilities.

### Materials and Methods:

The Bronchial Epithelial line Calu-3 was used in Patch clamp studies. Calu-3 Bronchial Epithelial cells (ATCC #HTB-55) were grown in a 1:1 mixture of Ham's F12 and DMEM medium that was supplemented with 10% FBS onto glass coverslips until the time of the experiments. In brief, a whole cell voltage clamp device was used to measure effects on Calu-3 cells exposed to the inventive electrokinetically generated fluids (e.g., RNS-60; electrokinetically treated normal saline comprising 60 ppm dissolved oxygen; sometimes referred to as “drug”).

Patch clamping techniques were utilized to assess the effects of the test material (RNS-60) on epithelial cell membrane polarity and ion channel activity. Specifically, whole cell voltage clamp was performed upon the Bronchial Epithelial line Calu-3 in a bathing solution consisting of: 135 mM NaCl, 5 mM KCl, 1.2 mM CaCl2, 0.8 mM MgCl2, and 10 mM HEPES (pH adjusted to 7.4 with N-methyl D-Glucamine). Basal currents were measured after which RNS-60 was perfused onto the cells.

More specifically, patch pipettes were pulled from borosilicate glass (Garner Glass Co, Claremont, Calif.) with a two-stage Narishige PB-7 vertical puller and then fire-polished to a resistance between 6-12 Mohms with a Narishige MF-9 microforge (Narishige International USA, East Meadow, N.Y.). The pipettes were filled with an intracellular solution containing (in mM): 135 KCl, 10 NaCl, 5 EGTA, 10 Hepes, pH was adjusted to 7.4 with NMDG (N-Methyl-D-Glucamine).

The cultured Calu-3 cells were placed in a chamber containing the following extracellular solution (in mM): 135 NaCl, 5 KCl, 1.2 CaCl2, 0.5 MgCl2 and 10 Hepes (free acid), pH was adjusted to 7.4 with NMDG.

Cells were viewed using the 40×DIC objective of an Olympus IX71 microscope (Olympus Inc., Tokyo, Japan). After a cell-attached gigaseal was established, a gentle suction was applied to break in, and to attain the whole-cell configuration. Immediately upon breaking in, the cell was voltage clamped at −120, −60, −40 and 0 mV, and was stimulated with voltage steps between ±100 mV (500 ms/step). After collecting the whole-cell currents at the control condition, the same cell was perfused through bath with the test fluid comprising same extracellular solutes and pH as for the above control fluid, and whole-cell currents at different holding potentials were recorded with the same protocols.

Electrophysiological data were acquired with an Axon Patch 200B amplifier, low-pass filtered at 10 kHz, and digitized with 1400A Digidata (Axon Instruments, Union City, Calif.). The pCLAMP 10.0 software (Axon Instruments) was used to acquire and to analyze the data. Current (I)-to-voltage (V) relationships (whole cell conductance) were obtained by plotting the actual current value at approximately 400 msec into the step, versus the holding potential (V). The slope of the I/V relationship is the whole cell conductance.

Drugs and Chemicals. Whenever indicated, cells were stimulated with a cAMP stimulatory cocktail containing 8-Br-cAMP (500 mM), IBMX (isobutyl-1-methylxanthine, 200 mM) and forskolin (10 mM). The cAMP analog 8-Br-cAMP (Sigma Chem. Co.) was used from a 25 mM stock in H2O solution. Forskolin (Sigma) and IBMX (Sigma) were used from a DMSO solution containing both 10 mM Forskolin and 200 mM IBMX stock solution.

### Patch Clamp Results:

Applicants determined whole-cell currents under basal (no cAMP) conditions, with a protocol stepping from zero mV holding potential to +/−100 mV. Representative tracings (control, followed by the whole-cell tracings while perfusing the test solution) were made on an average of n=12 cells. Composite ‘delta’ tracings, obtained by subtraction of the test average values, from those under control conditions were obtained. The whole-cell conductance, obtained from the current-to-voltage relationships was highly linear under both conditions, and reflects a modest, albeit significant change in conductance due to the test conditions. The contribution to the whole-cell conductance, i.e., the component inhibited by the drug (inventive electrokinetically generated fluid) was also linear, and the reversal potential was near zero mV. There was a decrease in the whole cell conductance under hyperpolarizing conditions.

In addition, Applicant determined whole-cell currents under basal conditions, with a protocol stepping from −40 mV holding potential to ±100 mV. Representative tracings (control, followed by the whole-cell tracings while perfusing the test solution) were made on an average of n=12 cells. Composite delta tracings were obtained by subtraction of the test average values, from those under control conditions. The whole-cell conductance obtained from the current-to-voltage relationships was highly linear under both conditions, and reflected a modest, albeit significant change in conductance due to the test conditions. The contribution to the whole-cell conductance, i.e., the component inhibited by the drug (inventive electrokinetically generated fluid) was also linear, and the reversal potential was near zero mV. Values were comparatively similar to those obtained with the zero mV protocol.

Applicants determined whole-cell currents under basal conditions, with a protocol stepping from −60 mV holding potential to ±100 mV. Representative tracings (control, followed by the whole-cell tracings while perfusing the test solution) were made on an average of n=12 cells. Composite ‘delta’ tracings were obtained by subtraction of the test average values, from those under control conditions. The whole-cell conductance obtained from the current-to-voltage relationships was highly linear under both conditions, and reflected a minor, albeit significant change in conductance due to the test conditions. The contribution to the whole-cell conductance, i.e., the component inhibited by the drug is also linear, and the reversal potential was near zero mV. Values were comparatively similar to those obtained with the zero mV protocol.

Applicants also determined whole-cell currents under basal conditions, with a protocol stepping from −120 mV holding potential to ±100 mV. Representative tracings (control, followed by the whole-cell tracings while perfusing the test solution) were made on an average of n=12 cells. Composite ‘delta’ tracings were obtained by subtraction of the test average values, from those under control conditions. The whole-cell conductance obtained from the current-to-voltage relationships was highly linear under both conditions, and refleced a minor, albeit significant change in conductance due to the test conditions. The contribution to the whole-cell conductance, i.e., the component inhibited by the drug is also linear, and the reversal potential was near zero mV. Values were comparatively similar to those obtained with the zero mV protocol.

In addition, Applicants determined whole-cell currents under cAMP-stimulated conditions, obtained with protocols stepping from various holding potentials to ±100 mV. Representative tracings are the average of n=5 cells. Representative tracings (control, followed by the whole-cell tracings after cAMP stimulation, followed by perfusion with the drug-containing solution) were made on an average of n=12 cells. Composite ‘delta’ tracings (corresponding to voltage protocols at zero mV, and at −40 mV) were obtained by subtraction of the test average values in drug+cAMP, from those under control conditions (cAMP alone). The whole-cell conductance obtained from the current-to-voltage relationships was highly linear under all conditions, and reflected a change in conductance due to the test conditions.

Applications demonstrated whole-cell currents under cAMP-stimulated conditions, obtained with protocols stepping from various holding potentials to ±100 mV. Representative tracings (control, followed by the whole-cell tracings after cAMP stimulation, followed by perfusion with the drug-containing solution) were made on a average of n=5 cells. Composite ‘delta’ tracings (voltage protocols at −60 mV, and −120 mV) were obtained by subtraction of the test average values in drug+cAMP, from those under control conditions (cAMP alone). The whole-cell conductance, obtained from the current-to-voltage relationships, was highly linear under all conditions, and reflected a change in conductance due to the test conditions.

Applicants also demonstrated the effect of holding potential on cAMP-activated currents. The effect of the drug (the inventive electrokinetically generated fluids; RNS-60; electrokinetically treated normal saline comprising 60 ppm dissolved oxygen) on the whole-cell conductance was observed under different voltage protocols (0, −40, −60, −120 mV holding potentials). Under basal conditions, the drug-sensitive whole-cell current was identical at all holding potentials (voltage-insensitive contribution). In the cAMP-activated conditions, however, the drug-sensitive currents were much higher, and sensitive to the applied voltage protocol. The current-to-voltage relationships are highly nonlinear. This was further observed in the subtracted currents, where the contribution of the whole cell conductance at zero mV was further subtracted for each protocol (n=5).

Summary of Example. According to particular aspects, therefore, the data indicate that there is a modest but consistent effect of the drug (the inventive electrokinetically generated fluids; RNS-60; electrokinetically treated normal saline comprising 60 ppm dissolved oxygen) under basal conditions. To enhance the effect of the drug on the whole-cell conductance, experiments were also conducted by perfusing the drug after stimulation with a cAMP stimulating “cocktail”, which dramatically increased the whole-cell conductance. Interestingly, this protocol also increased the drug-sensitive portion of the whole-cell conductance, which was ten-times higher than that observed under basal conditions. Additionally, in the presence of cAMP stimulation, the drug showed different effects with respect to the various voltage protocols, indicating that the electrokinetically generated fluids affect a voltage-dependent contribution of the whole-cell conductance. There was also a decrease in a linear component of the conductance, further suggesting at least a contribution of the drug to the inhibition of another pathway (e.g., ion channel, voltage gated cation channels, etc.).

In particular aspects, and without being bound by mechanism, Applicants' data are consistent with the inventive electrokinetically generated fluids (e.g., RNS-60; electrokinetically treated normal saline comprising 60 ppm dissolved oxygen) producing a change either on a channel(s), being blocked or retrieved from the plasma membrane.

Taken together with Applicants' other data, particular aspects of the present invention provide compositions and methods for modulating intracellular signal transduction, including modulation of at least one of membrane structure, membrane potential or membrane conductivity, membrane proteins or receptors, ion channels, and calcium dependant cellular signaling systems, comprising use of the inventive electrokinetically generated solutions to impart electrochemical and/or conformational changes in membranous structures (e.g., membrane and/or membrane proteins, receptors or other components) including but not limited to GPCRs and/or g-proteins, and TSLP. According to additional aspects, these effects modulate gene expression, and may persist, dependant, for example, on the half lives of the individual messaging components, etc.

### Example 7

### Effects of Inventive Electrokinetically-Altered Fluids on Whole-Cell Conductance were Determined

Overview. Patch clamp analysis conducted on Calu-3 cells perfused with inventive electrokinetically generated fluids (RNS-60 and Solas) revealed that (i) exposure to RNS-60 and Solas resulted in increases in whole cell conductance, (ii) that exposure of cells to the RNS-60 produced an increase in a non-linear conductance, evident at 15 min incubation times, and (iii) that exposure of cells to the RNS-60 produced an effect of RNS-60 saline on calcium permeable channels. Applicants performed patch clamp studies to further confirm the utilities, as described herein, of the inventive electrokinetically generated saline fluids (RNS-60 and Solas), including the utility to modulate whole-cell currents. Two sets of experiments were conducted.

The summary of the data of the first set of experiments indicates that the whole cell conductance (current-to-voltage relationship) obtained with Solas saline is highly linear for both incubation times (15 min, 2 hours), and for all voltage protocols. It is however evident, that longer incubation (2 hours) with Solas increased the whole cell conductance. Exposure of cells to the RNS-60 produced an increase in a non-linear conductance, as shown in the delta currents (Rev-Sol subtraction), which is only evident at 15 min incubation time. The effect of the RNS-60 on this non-linear current disappears, and is instead highly linear at the two-hour incubation time. The contribution of the non-linear whole cell conductance, as previously observed, was voltage sensitive, although present at all voltage protocols.

The summary of data of the second set of experiments indicates that there is an effect of the RNS-60 saline on a non-linear current, which was made evident in high calcium in the external solution. The contribution of the non-linear whole cell conductance, although voltage sensitive, was present in both voltage protocols, and indicates an effect of RNS-60 saline on calcium permeable channels.

### First Set of Experiments (Increase of Conductance and Activation of a Non-Linear Voltage Regulated Conductance)

Methods for first set of experiments. See above for general patch clamp methods. In the following first set of experiments, patch clamp studies were performed to further confirm the utility of the inventive electrokinetically generated saline fluids (RNS-60 and Solas) to modulate whole-cell currents, using Calu-3 cells under basal conditions, with protocols stepping from either zero mV holding potential, −120 mV, or −60 mV.

The whole-cell conductance in each case was obtained from the current-to-voltage relationships obtained from cells incubated for either 15 min or two hour. In this study, groups were obtained at a given time, for either Solas or RNS-60 saline solutions. The data obtained are expressed as the mean±SEM whole cell current for 5-9 cells.

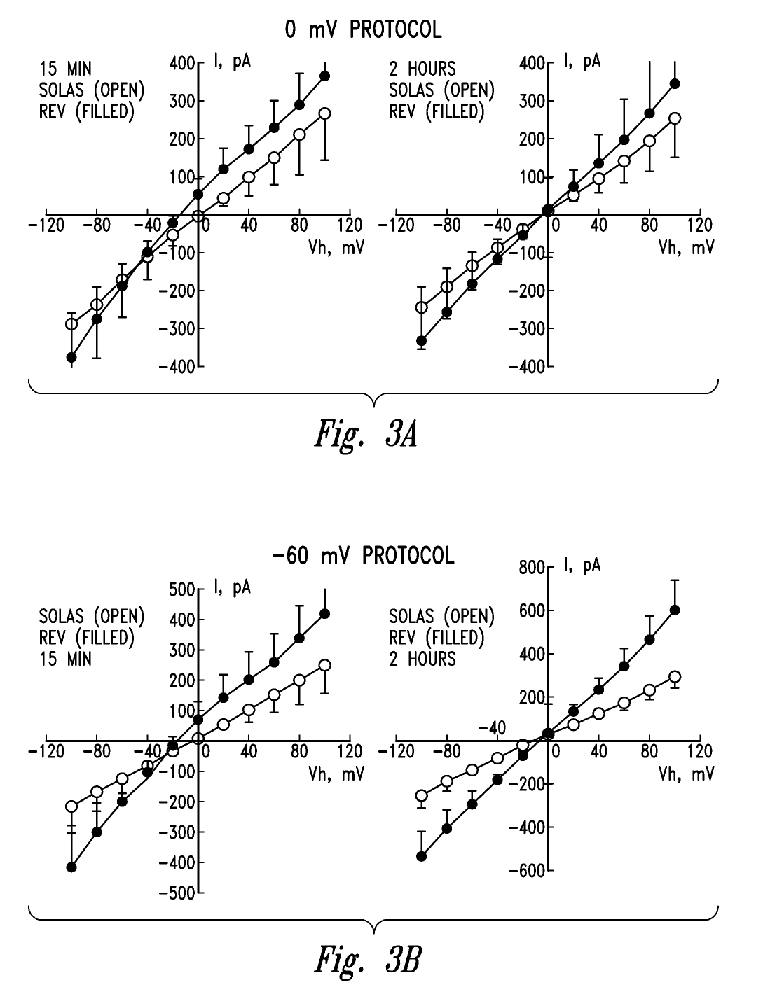

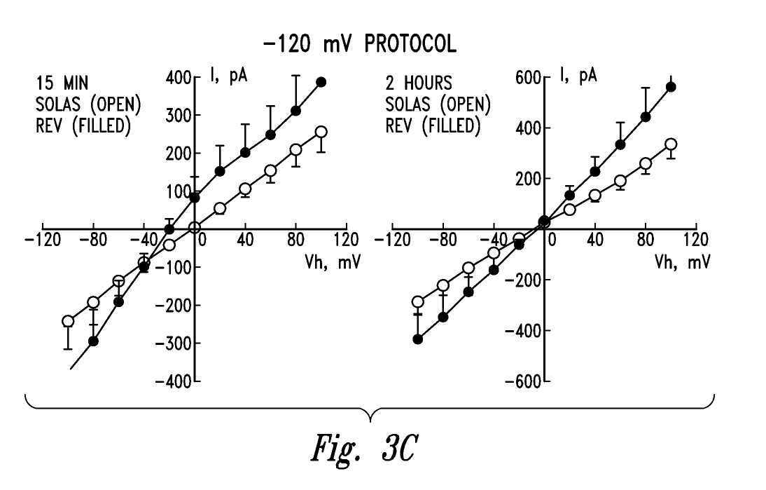

Results. FIGS. 3 A-C show the results of a series of patch clamping experiments that assessed the effects of the electrokinetically generated fluid (e.g., RNS-60 and Solas) on epithelial cell membrane polarity and ion channel activity at two time-points (15 min (left panels) and 2 hours (right panels)) and at different voltage protocols (A, stepping from zero mV; B, stepping from −60 mV; and C, stepping from −120 mV). The results indicate that the RNS-60 (filled circles) has a larger effect on whole-cell conductance than Solas (open circles). In the experiment similar results were seen in the three voltage protocols and at both the 15 minute and two-hour incubation time points.

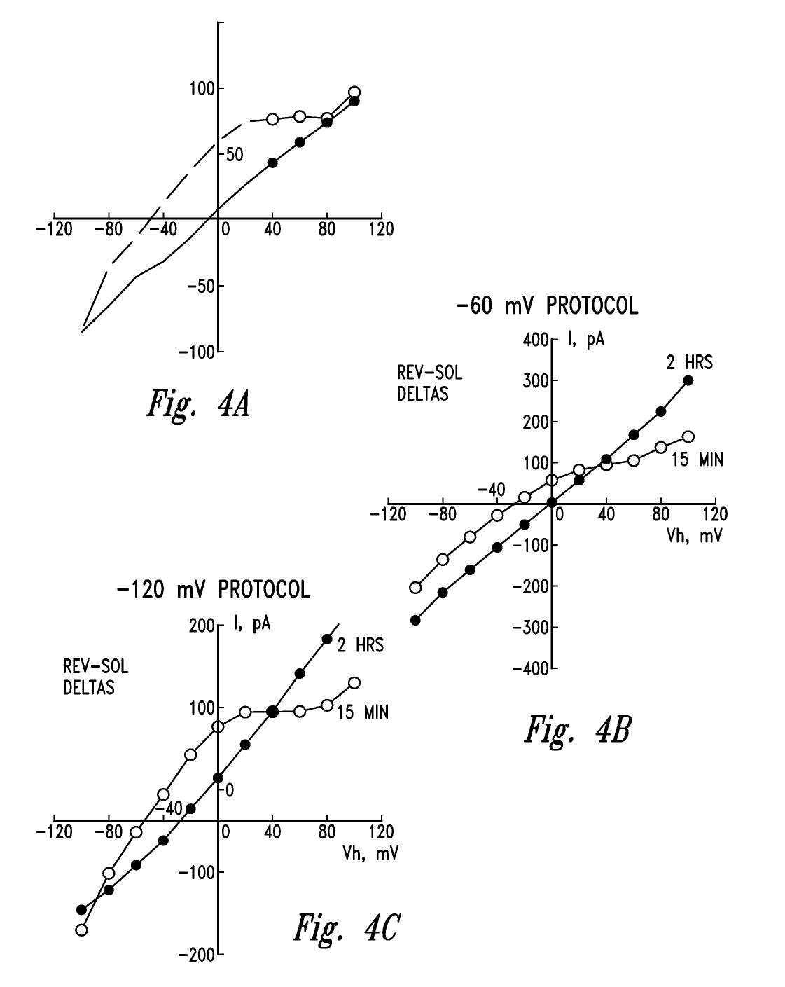

FIGS. 4 A-C show graphs resulting from the subtraction of the Solas current data from the RNS-60 current data at three voltage protocols (“Delta currents”) (A, stepping from zero mV; B, stepping from −60 mV; and C, stepping from −120 mV) and the two time-points (15 mins (open circles) and 2 hours (filled circles)). These data indicated that at the 15 minute time-point with RNS-60, there is a non-linear voltage-dependent component that is absent at the 2 hour time point.

As in previous experiments, data with “Normal” saline gave a very consistent and time-independent conductance used as a reference. The present results were obtained by matching groups with either Solas or RNS-60 saline, and indicate that exposure of Calu-3 cells to the RNS-60 saline under basal conditions (without cAMP, or any other stimulation), produces time-dependent effect(s), consistent with the activation of a voltage-regulated conductance at shorter incubation times (15 min). This phenomenon was not as apparent at the two-hour incubation point. As described elsewhere herein, the linear component is more evident when the conductance is increased by stimulation with the cAMP “cocktail”. Nonetheless, the two-hour incubation time showed higher linear conductance for both the RNS-60 and the Solas saline, and in this case, the RNS-60 saline doubled the whole cell conductance as compared to Solas alone. This evidence indicates that at least two contributions to the whole cell conductance are affected by the RNS-60 saline, namely the activation of a non-linear voltage regulated conductance, and a linear conductance, which is more evident at longer incubation times.

### Second Set of Experiments (Effect on Calcium Permeable Channels)

Methods for second set of experiments. See above for general patch clamp methods. In the following second set of experiments, yet additional patch clamp studies were performed to further confirm the utility of the inventive electrokinetically generated saline fluids (RNS-60 and Solas) to modulate whole-cell currents, using Calu-3 cells under basal conditions, with protocols stepping from either zero mV or −120 mV holding potentials.

The whole-cell conductance in each case was obtained from the current-to-voltage relationships obtained from cells incubated for 15 min with either saline. To determine whether there is a contribution of calcium permeable channels to the whole cell conductance, and whether this part of the whole cell conductance is affected by incubation with RNS-60 saline, cells were patched in normal saline after the incubation period (entails a high NaCl external solution, while the internal solution contains high KCl). The external saline was then replaced with a solution where NaCl was replaced by CsCl to determine whether there is a change in conductance by replacing the main external cation. Under these conditions, the same cell was then exposed to increasing concentrations of calcium, such that a calcium entry step is made more evident.

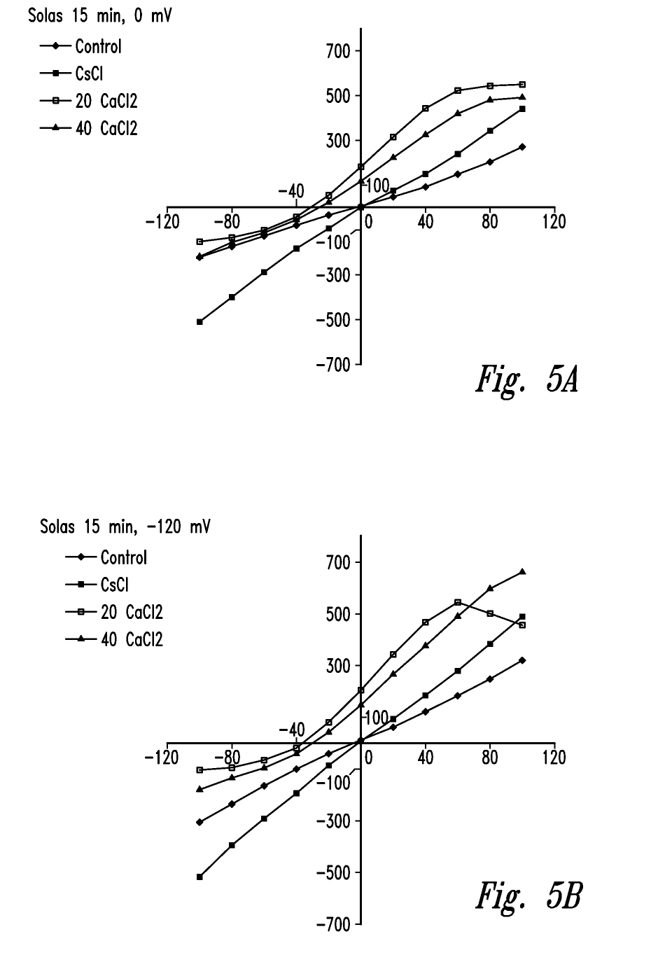

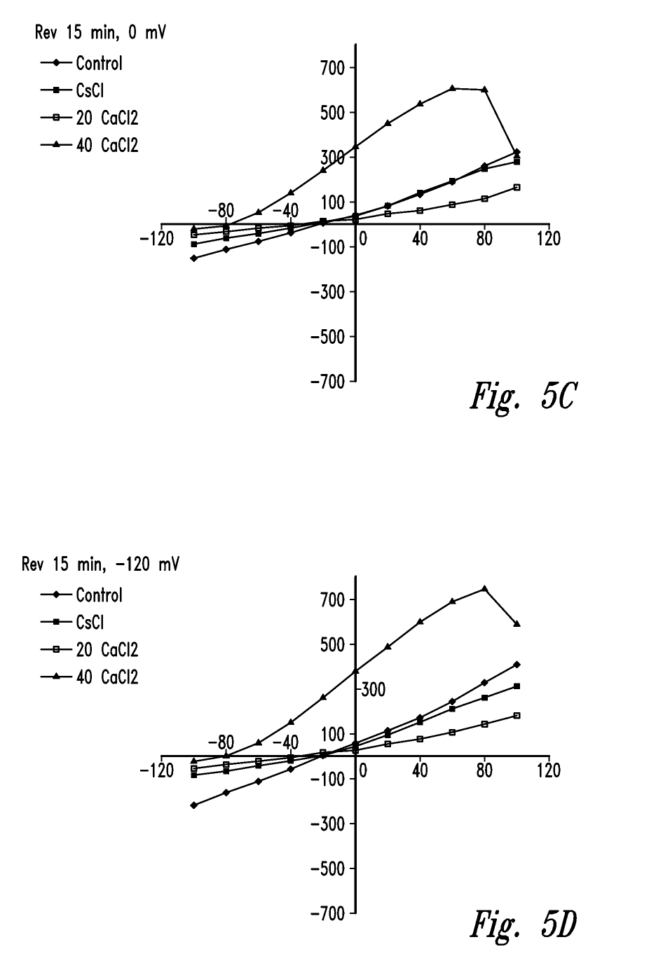

Results: FIGS. 5 A-D show the results of a series of patch clamping experiments that assessed the effects of the electrokinetically generated fluid (e.g., Solas (panels A and B) and RNS-60 (panels C and D)) on epithelial cell membrane polarity and ion channel activity using different external salt solutions and at different voltage protocols (panels A and C show stepping from zero mV, whereas panels B and D show stepping from −120 mV). In these experiments one time-point of 15 minutes was used. For Solas (panels A and B) the results indicate that: 1) using CsCl (square symbols) instead of NaCl as the external solution, increased whole cell conductance with a linear behavior when compared to the control (diamond symbols); and 2) CaCl₂ at both 20 mM CaCl₂ (circle symbols) and 40 mM CaCl₂ (triangle symbols) increased whole cell conductance in a non-linear manner. For RNS-60 (panels C and D), the results indicate that: 1) using CsCl (square symbols) instead of NaCl as the external solution had little effect on whole cell conductance when compared to the control (diamond symbols); and 2) CaCl₂ at 40 mM (triangle symbols) increased whole cell conductance in a non-linear manner.

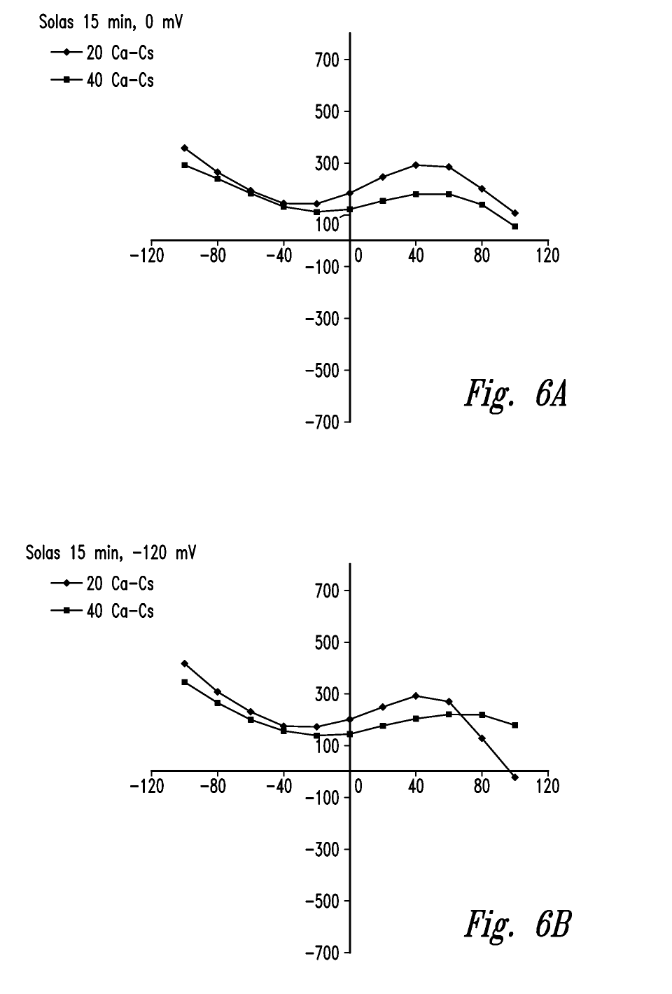

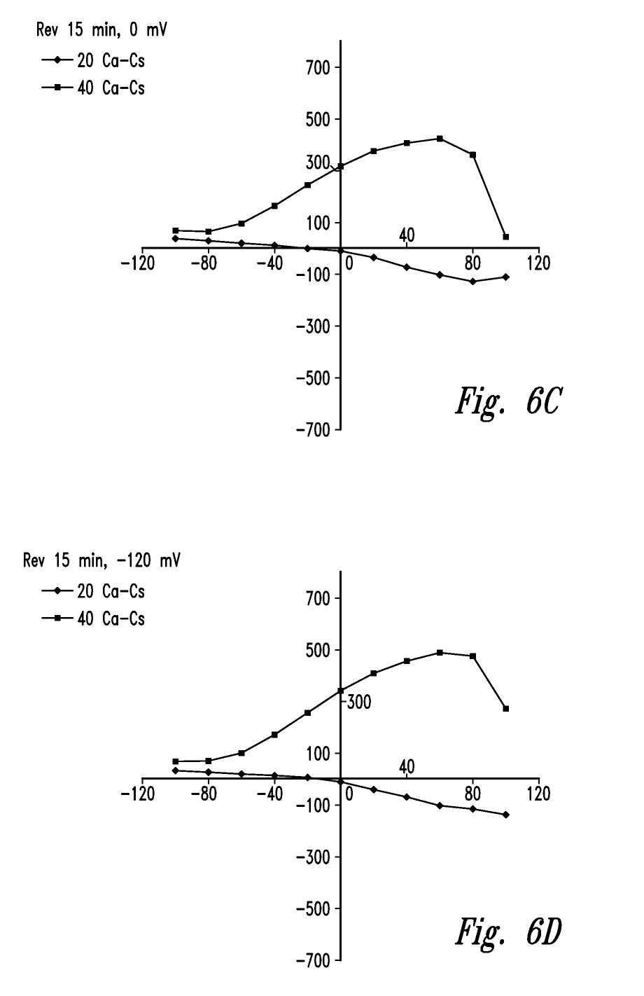

FIGS. 6 A-D show the graphs resulting from the subtraction of the CsCl current data (shown in FIG. 5) from the 20 mM CaCl₂ (diamond symbols) and 40 mM CaCl₂ (square symbols) current data at two voltage protocols (panels A and C, stepping from zero mV; and B and D, stepping from −120 mV) for Solas (panels A and B) and RNS-60 (panels C and D). The results indicate that both Solas and RNS-60 solutions activated a calcium-induced non-linear whole cell conductance. The effect was greater with RNS-60 (indicating a dosage responsiveness), and with RNS-60 was only increased at higher calcium concentrations. Moreover, the non-linear calcium dependent conductance at higher calcium concentration was also increased by the voltage protocol.

The data of this second set of experiments further indicates an effect of RNS-60 saline and Solas saline for whole cell conductance data obtained in Calu-3 cells. The data indicate that 15-min incubation with either saline produces a distinct effect on the whole cell conductance, which is most evident with RNS-60, and when external calcium is increased, and further indicates that the RNS-60 saline increases a calcium-dependent non-linear component of the whole cell conductance.

The accumulated evidence suggests activation by Revalesio saline of ion channels, which make different contributions to the basal cell conductance.

Taken together with Applicants' other data (e.g., the data of Applicants other working Examples) particular aspects of the present invention provide compositions and methods for modulating intracellular signal transduction, including modulation of at least one of membrane structure, membrane potential or membrane conductivity, membrane proteins or receptors, ion channels, lipid components, or intracellular components with are exchangeable by the cell (e.g., signaling pathways, such as calcium dependant cellular signaling systems, comprising use of the inventive electrokinetically generated solutions to impart electrochemical and/or conformational changes in membranous structures (e.g., membrane and/or membrane proteins, receptors or other membrane components) including but not limited to GPCRs and/or g-proteins. According to additional aspects, these effects modulate gene expression, and may persist, dependant, for example, on the half lives of the individual messaging components, etc.

### Example 8

### Effects of Inventive Electrokinetically-Altered Fluids on Whole-Cell Conductance were Investigated, and a Dose Response Curve was Generated

Overview. In this experiment Applicants assessed the effect of dilutions of the electrokinetically-altered fluid (e.g., RNS-60) on epithelial cell membrane polarity and ion channel activity.

Methods. See above for general patch clamp methods. In the following experiment, patch clamp studies were performed to further confirm the utility of the inventive electrokinetically generated saline fluids (RNS-60) to modulate whole-cell currents. In particular, the experiment assessed the effect of dilutions of the inventive electrokinetically generated saline fluid. The solutions were made by diluting the inventive electrokinetically generated saline fluid in normal saline at concentrations of: 100% (Rev), 75% (3:4), 50% (1:1), 25% (4:3), and 0% (Sal).

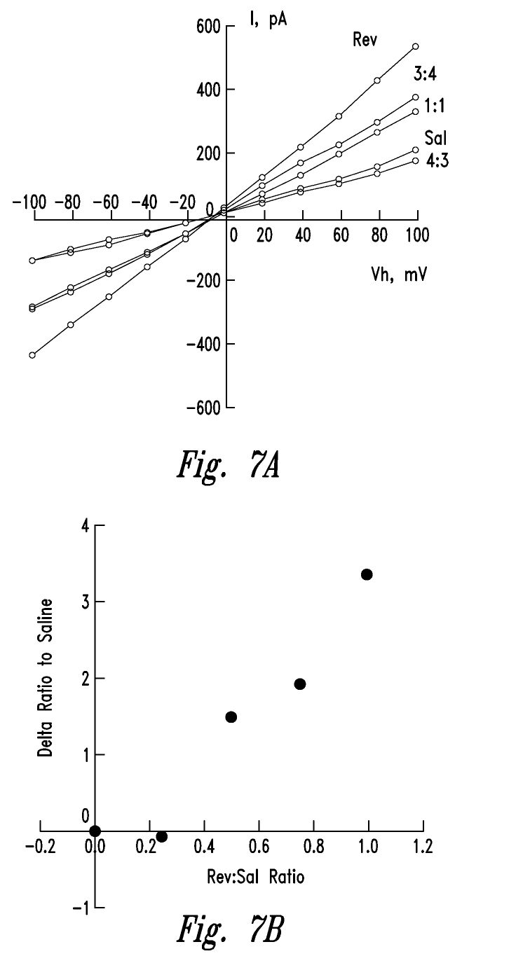

Results. FIGS. 7 A and B show the results of a series of patch clamp experiments that assessed the effects of diluted electrokinetically generated fluid (e.g., RNS-60) on epithelial cell membrane polarity and ion channel activity. Panel A demonstrates the volts versus current of whole cell conductance for each diluted sample as indicated on the graph (Rev, 3:4, 1:1, 4:3, and Sal). Panel B demonstrates the dilution amount versus the change in current comparing the dilution to normal saline. The results indicate that the mechanism of action of the RNS-60 solution occurs in a linear dose responsive manner.

### Example 9

### Treatment of Primary Bronchial Epithelial Cells (BEC) with the Inventive Electrokinetically Generated Fluids, as Well as with Non-Electrokinetic Control Pressure Pot Fluid, Resulted in Reduced expression and/or activity of two key proteins of the airway inflammatory pathways, MMP9 and TSLP

Overview. Applicants have now shown (using Bio-Layer Interferometry biosensor, Octet Rapid Extended Detection (RED) (forteBio™)), that in the presence of electrokinetically generated fluids (e.g., Rev; gas-enriched electrokinetically generated fluid) of the instant disclosure compared to normal saline, Bradykinin binding to the B2 receptor was concentration dependent, and binding affinity was increased. Additionally, in the context of T-regulatory cells stimulated with diesel exhaust particulate matter (PM, standard commercial source), Applicants' data showed a decreased proliferation of T-regulatory cells in the presence of PM and Rev relative to PM in control fluid (no Rev, no Solis), indicating that the inventive electrokinetically generated fluid Rev improved regulatory T-cell function; e.g., as shown by relatively decreased proliferation in the assay. Moreover, exposure to the inventive fluids resulted in a maintained or only slightly decreased production of IL-10 relative to the Saline and Media controls (no PM). Likewise, in the context of the allergic asthma (AA) profiles of peripheral blood mononuclear cells (PBMC) stimulated with particulate matter (PM), the data showed that exposure to the fluids of the instant disclosure (“PM+Rev”) resulted in significantly lower tryptase levels similar to those of the Saline and Media controls. Additionally, Diptheria toxin (DT390, a truncated diphtheria toxin molecule; 1:50 dilution of std. commercial concentration) resulted in beta blockade, GPCR blockade and Ca channel blockade of the effects the activity of the electrokinetically generated fluids on Treg and PBMC function. Furthermore, Applicants' has shown that upon exposure to the inventive fluids, tight junction related proteins (e.g., JAM 2 and 3, GJA1, 3, 4 and 5 (junctional adherins), OCLN (occludin), claudins (e.g., CLDN 3, 5, 7, 8, 9, 10), TJP1 (tight junction protein 1)) were upregulated in lung tissue. Furthermore, as shown in patch clamp studies, the inventive electrokinetically generated fluids (e.g., RNS-60) affect modulation of whole cell conductance (e.g., under hyperpolarizing conditions) in Bronchial Epithelial Cells (BEC; e.g., Calu-3), and according to additional aspects, modulation of whole cell conductance reflects modulation of ion channels.

In this Example, Applicants have extended these discoveries by conducting additional experiments to measure the effects of production of two key proteins of the airway inflammatory pathways. Specifically, MMP9 and TSLP were assayed in primary bronchial epithelial cells (BEC).

### Materials and Methods:

Commercially available primary human bronchial epithelial cells (BEC) (HBEpC-c from Promocell, Germany) were used for these studies. Approximately 50,000 cells were plated in each well of a 12 well plate until they reached ˜80% confluence. The cells were then treated for 6 hours with normal saline, control fluid Solas, non-electrokinetic control pressure pot fluid, or the test fluid Revera 60 at a 1:10 dilution (100 ul in 1 ml of airway epithelial growth medium) along with the diesel exhaust particulate matter (DEP or PM) before being lifted for FACS analysis. Both MMP9 and TSLP receptor antibodies were obtained from BD Biosciences and used as per manufacturer's specifications.

### Results:

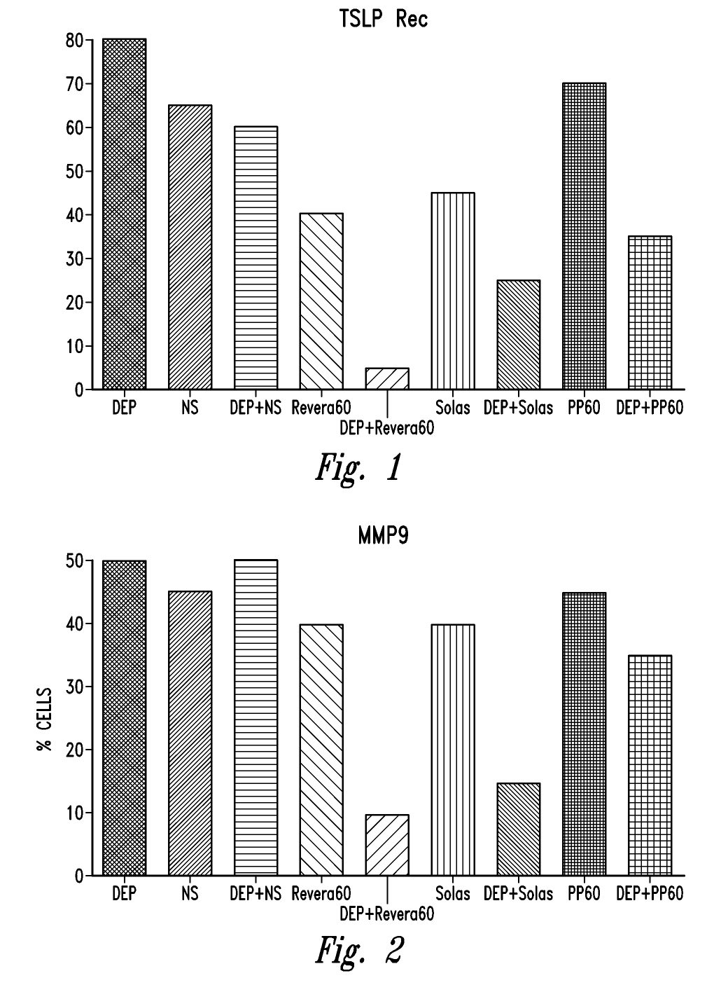

In FIGS. 1 and 2, DEP represents cells exposed to diesel exhaust particulate matter (PM, standard commercial source) alone, “NS” represents cells exposed to normal saline alone, “DEP+NS” represent cells treated with particulate matter in the presence of normal saline, “Revera 60” refers to cells exposed only to the test material, “DEP+Revera 60” refer to cells treated with particulate matter in the presence of the test material Revera 60. In addition, “Solas” and “DEP+Solas” represents cells exposed to the control fluid Solas alone or in combination with the particulate matter, respectively. “PP60” represents cells exposed to the non-electrokinetic control pressure pot fluid, and “DEP+PP60” refers to cells treated with particulate matter in the presence of the non-electrokinetic control pressure pot fluid (i.e., having 60 ppm dissolved oxygen).

FIG. 1 shows that the test material Revera 60 reduces DEP induced TSLP receptor expression in bronchial epithelial cells (BEC) by approximately 90%. Solas resulted in a 55% reduction in DEP induced TSLP receptor expression, while Normal Saline failed to produce similar level of reduction in DEP induced TSLP receptor expression (approximately 20% reduction). Additionally, the non-electrokinetic control pressure pot fluid PP60 resulted in approximately 50% reduction in DEP induced TSLP receptor expression.

The effect of the inventive Revera 60, Solas, and also of the PP60 solutions in reducing TSLP receptor expression is a significant discovery in view of recent findings showing that TSLP plays a pivotal role in the pathobiology of allergic asthma and local antibody mediated blockade of TSLP receptor function alleviated allergic disease (Liu, Y J, Thymic stromal lymphopoietin: Master switch for allergic inflammation, J Exp Med 203:269-273, 2006; Al-Shami et al., A role for TSLP in the development of inflammation in an asthma model, J Exp Med 202:829-839, 2005; and Shi et al., Local blockade of TSLP receptor alleviated allergic disease by regulating airway dendritic cells, Clin Immunol. 2008, Aug. 29. (Epub ahead of print)).

Likewise, FIG. 2 shows the effect of Revera 60, Solas, non-electrokinetic control pressure pot fluid (PP60), and normal saline on the DEP-mediated increase in MMP 9. Specifically, Revera 60 inhibited the DEP-induced cell surface bound MMP9 levels in bronchial epithelial cells by approximately 80%, and Solas had an inhibitory effect of approximately 70%, whereas normal saline (NS) had a marginal effect of about 20% reduction. Additionally, the non-electrokinetic control pressure pot fluid PP60 resulted in approximately 30% reduction in DEP-induced cell surface attached MMP9 levels. MMP-9 is one of the major proteinases involved in airway inflammation and bronchial remodeling in asthma. Recently, it has been demonstrated that the levels of MMP-9 are significantly increased in patients with stable asthma and even higher in patients with acute asthmatic patients compared with healthy control subjects. MMP-9 plays a crucial role in the infiltration of airway inflammatory cells and the induction of airway hyperresponsiveness indicating that MMP-9 may have an important role in inducing and maintaining asthma (Vignola et al., Sputum metalloproteinase-9/tissue inhibitor of metalloproteinase-1 ratio correlates with airflow obstruction in asthma and chronic bronchitis, Am J Respir Crit Care Med 158:1945-1950, 1998; Hoshino et al., Inhaled corticosteroids decrease subepithelial collagen deposition by modulation of the balance between matrix metalloproteinase-9 and tissue inhibitor of metalloproteinase-1 expression in asthma, J Allergy Clin Immunol 104:356-363, 1999; Simpson et al., Differential proteolytic enzyme activity in eosinophilic and neutrophilic asthma, Am J Respir Crit Care Med 172:559-565, 2005; Lee et al., A murine model of toluene diisocyanate-induced asthma can be treated with matrix metalloproteinase inhibitor, J Allergy Clin Immunol 108:1021-1026, 2001; and Lee et al., Matrix metalloproteinase inhibitor regulates inflammatory cell migration by reducing ICAM-1 and VCAM-1 expression in a murine model of toluene diisocyanate-induced asthma, J Allergy Clin Immunol 2003; 111:1278-1284).

According to additional aspects, therefore, the inventive electrokinetically generated fluids have substantial therapeutic utility for modulating (e.g., reducing) TSLP receptor expression and/or for inhibiting expression and/or activity of MMP-9, including, for example, for treatment of inflammation and asthma.

According to yet additional aspects, non-electrokinetic control pressure pot fluid (i.e., having 60 ppm dissolved oxygen) have therapeutic utility for modulating (e.g., reducing) TSLP receptor expression and/or for inhibiting expression and/or activity of MMP-9, including, for example, for treatment of inflammation and asthma. Without being bound by mechanism, Applicants' collective data indicates that the action of the non-electrokinetic control pressure pot fluid in this system is mediated by a mechanism that is distinct from that of Applicants' electrokinetically-generated fluids. This is not only because the effects are relatively smaller, but also because non-electrokinetic control pressure pot fluid has not displayed activity in other assays displaying activity with Applicants' electrokinetically generated fluids. Nonetheless, Applicants' discovery of the herein disclosed activity of non-electrokinetic control pressure pot fluid in this system represents a novel use for such pressure pot fluid in the context of asthma and related conditions as disclosed herein.

According to particular aspects, therefore, the inventive methods comprising administration of Applicants' electrokinetically generated fluids provide for modulation (down-regulation of TSLP expression and/or activity) are applicable to the treatment of at least one disease or condition selected from the TSLP-mediated group consisting of disorders of the immune system, allergic inflammation, allergic airway inflammation, DC-mediated inflammatory Th2 responses, atopic dermatitis, atopic eczema, asthma, obstructive airways disease, chronic obstructive pulmonary disease, and food allergies, inflammatory arthritis, rheumatoid arthritis and psoriasis.

The results disclosed herein are entirely consistent with the art-recognized role of TSLP as a master switch of allergic inflammation at the epithelial cell-DC interface (Yong-Jun et al., J. Exp. Med., 203:269-273, 2006), and are further consistent with the phenotypes of mice lacking the TSLPR (e.g., fail to develop asthma in response to inhaled antigens; Zhou et al., supra and Al-Shami et al., J. Exp. Med., 202:829-839, 2005), and with results obtained from pretreating OVA-DCs with anti-TSLPR (e.g., resulting in a significant reduction of eosinophils and lymphocyte infiltration as well as IL-4 and IL-5 levels.

The presently disclosed subject matter further illuminates the role that TSLPR plays in DC-primed allergic disease, and provides for novel compositions and methods comprising administration of Applicants' electrokinetically generated fluids.

### Example 10

### Effects of the Electrokinetic Fluids on Wound Healing were Determined

The effects of a gas-enriched fluid (enriched with oxygen) were tested for the ability of cultured human epidermal keratinocytes to seal a wound. The results disclosed in this Example are also disclosed in Applicants' published applications US 2008/0139674 and WO 2008/115290.

Human epidermal keratinocytes were isolated from neonatal foreskins that were obtained from routine circumcision and de-identified. Foreskins were washed twice in PBS and incubated in 2.4 U/mL Dispase II in order to separate the dermis from the epidermis. The epidermis was incubated with 0.25% trypsin/1 mM EDTA, neutralized with soy bean trypsin inhibitor, agitated, and passed through a 70 um sieve to separate the cells. Next, the cell suspension was centrifuged and resuspended in cell culture medium (M154) supplemented with 0.07 mM CaCl₂, and human keratinocyte growth supplements (0.2% hydrocortisone, 0.2 ng/mL human epidermal growth factor) and penicillin/streptomycin, amphoteracin antibiotic cocktail. The keratinocyte cell suspensions were plated onto uncoated 12-well culture dishes and the medium replaced after 24 hours, and every 48 hours after the initial seeding.

Upon reaching cellular confluence, linear scratches were made with a sterile p1000 pipette tip, which resulted in a uniform cell-free wound. The monolayers were washed several times with Dulbecco's PBS in order to remove any cellular debris. The wound monolayers were then incubated in the following media: i) the complete growth media (as described above in this Example); ii) the complete growth media diluted 1:1 with a sheared version of saline without oxygen (control fluid that was processed using the disclosed diffuser device but without adding a gas); and iii) the complete growth media diluted 1:1 with oxygen-enriched saline. Each study was done in triplicate.

Prior to incubation, the wells were filled with the respective media and sealed by placing a 25×25 mm glass coverslip on top of each well. At 6, 12, 24, and 48 hours post-wounding, oxygen measurements were made, and cultures were imagined.

Results. Six hours post-wounding, the edges of the wounds in the saline and gas-enriched media were more ruffled than those in the media control that was processed with the diffuser device disclosed herein, but without the addition of a gas. Twelve hours post-wounding the edges of the wounds in all three media appeared uneven, with keratinocytes along the borders migrating toward the center of the wounds. Quantification of migrating keratinocytes revealed approximately the same level of keratinocyte migration in the saline and gas-enriched media.

### Example 11

### Effect of the Electrokinetic Fluids on Improved Wound Healing was Demonstrated

A study was performed to determine the improved healing characteristics of wounds that were exposed to an oxygen-enriched saline solution that was processed according to embodiments disclosed herein. In this experiment, bandages were placed on porcine dermal excision biopsy wounds. The bandages soaked in oxygen-enriched saline solution or a control group of bandages soaked in a saline solution that was not oxygen-enriched. Microscopically, several factors were evaluated by the study including: 1) epidermalization; 2) neovascularization; 3) epidermal differentiation; 4) mast cell migration; and 5) mitosis. The results disclosed in this Example are also disclosed in Applicants' published applications US 2008/0139674 and WO 2008/115290.

Results. Externally, the wounds appeared to heal at varying rates. The wounds treated with the oxygen-enriched saline solution showed an increase in wound healing at days 4 through 11. However, both wounds seemed to complete healing at approximately the same time. The study showed that between days 3 and 11, the new epidermis in wounds treated with the oxygen-enriched saline solution migrated at two to four times as fast as the epidermis of the wounds treated with the normal saline solution. The study also showed that between 15 and 22 days, the wound treated by the oxygen-enriched saline solution differentiated at a more rapid rate as evidenced by the earlier formation of more mature epidermal layers. At all stages, the thickening that occurs in the epidermis associated with normal healing did not occur within the wounds treated by the oxygen-enriched saline solution.

Without wishing to be bound by any particular theory, it is believed that the oxygen-enriched saline solution may increase the localized level of NO within the wounds. NO modulates growth factors, collagen deposition, inflammation, mast cell migration, epidermal thickening, and neovascularization in wound healing. Furthermore, nitric oxide is produced by an inducible enzyme that is regulated by oxygen.

Thus, while not wishing to be bound to any particular theory, the inventive gas-enriched fluid may stimulate NO production, which is in accordance with the spectrum of wound healing effects seen in these experiments.

The epidermis of the healing pigs experienced earlier differentiation in the oxygen-enriched saline group at days 15 through 22. In the case of mast cell migration, differences also occurred in early and late migration for the oxygen-enriched solution. A conclusive result for the level of mitosis was unascertainable due to the difficulty in staining.

The results indicated that the wound treated with the oxygen-enriched saline solution showed much greater healing characteristics than the untreated wound. In addition, the results show a greater differentiated epidermis with more normal epidermal/dermal contour.

### INCORPORATION BY REFERENCE

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

It should be understood that the drawings and detailed description herein are to be regarded in an illustrative rather than a restrictive manner, and are not intended to limit the invention to the particular forms and examples disclosed. On the contrary, the invention includes any further modifications, changes, rearrangements, substitutions, alternatives, design choices, and embodiments apparent to those of ordinary skill in the art, without departing from the spirit and scope of this invention, as defined by the following claims. Thus, it is intended that the following claims be interpreted to embrace all such further modifications, changes, rearrangements, substitutions, alternatives, design choices, and embodiments.

The foregoing described embodiments depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Accordingly, the invention is not limited except as by the appended claims.

1. A method for treating an MMP9-mediated condition or disease, comprising administration to a mammal in need thereof, a therapeutically effective amount of an electrokinetically altered aqueous fluid comprising an ionic aqueous solution of charge-stabilized oxygen-containing nanostructures substantially having an average diameter of less than about 100 nanometers and stably configured in the ionic aqueous fluid in an amount sufficient for treating an MMP9-mediated condition or disease.

2. The method of claim 1, wherein the charge-stabilized oxygen-containing nanostructures are stably configured in the ionic aqueous fluid in an amount sufficient to provide, upon contact of a living cell by the fluid, modulation of at least one of cellular membrane potential and cellular membrane conductivity.

3. The method of claim 1, wherein the charge-stabilized oxygen-containing nanostructures are the major charge-stabilized gas-containing nanostructure species in the fluid.

4. The method of claim 1, wherein the percentage of dissolved oxygen molecules present in the fluid as the charge-stabilized oxygen-containing nanostructures is a percentage selected from the group consisting of greater than: 0.01%, 0.1%, 1%, 5%; 10%; 15%; 20%; 25%; 30%; 35%; 40%; 45%; 50%; 55%; 60%; 65%; 70%; 75%; 80%; 85%; 90%; and 95%.

5. The method of claim 1, wherein the total dissolved oxygen is substantially present in the charge-stabilized oxygen-containing nanostructures.

6. The method of claim 1, wherein the charge-stabilized oxygen-containing nanostructures substantially have an average diameter of less than a size selected from the group consisting of: 90 nm; 80 nm; 70 nm; 60 nm; 50 nm; 40 nm; 30 nm; 20 nm; 10 nm; and less than 5 nm.

7. The method of claim 1, wherein the ionic aqueous solution comprises a saline solution.

8. The method of claim 1, wherein the fluid is superoxygenated.

9. The method of claim 1, wherein the fluid comprises a form of solvated electrons.

10. The method of claim 1, wherein alteration of the electrokinetically altered aqueous fluid comprises exposure of the fluid to hydrodynamically-induced, localized electrokinetic effects.

11. The method of claim 10, wherein, exposure to the localized electrokinetic effects comprises exposure to at least one of voltage pulses and current pulses.

12. The method of claim 10, wherein the exposure of the fluid to hydrodynamically-induced, localized electrokinetic effects, comprises exposure of the fluid to electrokinetic effect-inducing structural features of a device used to generate the fluid.

13. The method of claim 1, wherein the MMP9-mediated condition or disease comprises an obstructive airways disease.

14. The method of claim 13, wherein the wherein the obstructive airways disease comprises at least one of asthma and chronic obstructive pulmonary disease.

15. The method of claim 1, wherein the MMP9-mediated condition or disease comprises at least one of rheumatoid arthritis, osteoarthritis, atherosclerosis, cancer, and multiple sclerosis.

16. The method of claim 1, wherein the MMP9-mediated condition or disease comprises at least one disease or disorder of the peripheral or central nervous system characterized by persistent or sustained expression and/or activity of MMP9, selected from the group consisting of Alzheimer's disease, stroke/cerebral ischemia, head trauma, spinal cord injury, multiple sclerosis, amyotrophic lateral sclerosis, Huntington's disease, Parkinson's disease, migraine, cerebral amyloid angiopathy, AIDS, age-related cognitive decline; mild cognitive impairment and prion diseases in a mammal.

17. The method of claim 1, comprising combination therapy, wherein at least one additional therapeutic agent is administered to the patient.

18. The method of claim 17, wherein the at least one additional therapeutic agent comprises administration of an additional inhibitor of at least one MMP.

19. The method of claim 18, wherein the at least one MMP is selected from the group consisting of MMP-1, MMP-2, MMP-7, MMP-8, MMP-9, MMP-10, MMP-11, MMP-12, MMP-13, MMP-14, MMP-15, MMP-16, MMP-17, MMP-18, MMP-19 and MMP-20 MMP-1, MMP-2, MMP-3, MMP-7, MMP-8, MMP-9, MMP-10, MMP-11, MMP-12, MMP-13, MMP-14, MMP-15, MMP-16, MMP-17, MMP-18, MMP-19 and MMP-20.

20. The method of claim 17, wherein the at least one additional therapeutic agent is a TSLP and/or TSLPR antagonist.

21. The method of claim 21, wherein the TSLP and/or TSLPR antagonist is selected from the group consisting of neutralizing antibodies specific for TSLP and the TSLP receptor, soluble TSLP receptor molecules, and TSLP receptor fusion proteins, including TSLPR-immunoglobulin Fc molecules or polypeptides that encode components of more than one receptor chain.

22. The method of claim 17, wherein the at least one additional therapeutic agent is selected from the group consisting of: standard non-steroidal anti-inflammatory drugs (NSAID'S), piroxicam, diclofenac; a propionic acid, naproxen, flubiprofen, fenoprofen, ketoprofen and ibuprofen; a fenamate, mefenamic acid, indomethacin, sulindac, apazone; a pyrazolone, phenylbutazone; a salicylate, aspirin; an analgesic or intraarticular therapy, a corticosteroid; a hyaluronic acid, hyalgan, synvisc; an immune suppressant, cyclosporine, interferon; a TNF-.alpha. inhibitor, Enbrel™; low dose methotrexate, lefunimide, hydroxychloroquine, d-penicilamine, auranofin, parenteral gold and oral gold.

23. The method of claim 17, wherein the at least one additional therapeutic agent is selected from the CNS agent group consisting of: an antidepressant, sertraline, fluoxetine, paroxetine; an anti-Parkinsonian drug; deprenyl, L-dopa, requip, miratex; a MAOB inhibitor, selegine, rasagiline; a COMP inhibitor, tolcapone, Tasmar; an A-2 inhibitor, a dopamine reuptake inhibitor, an NMDA antagonist, a nicotine agonist, a dopamine agonist, an inhibitor of neuronal nitric oxide synthase, an anti-Alzheimer's drug; an acetylcholinesterase inhibitor, metrifonate, donepezil, Aricept, Exelon, ENA 713 or rivastigmine; tetrahydroaminoacridine, Tacrine, Cognex, or THA; a COX-1 or COX-2 inhibitor, celecoxib, Celebrex, rofecoxib, Vioxx; propentofylline, an anti-stroke medication, an NR2B selective antagonist, a glycine site antagonist, and a neutrophil inhibitory factor (NIF).

24. The method of claim 17, wherein the at least one additional therapeutic agent is selected from the group consisting of: an estrogen; a selective estrogen modulator, estrogen, raloxifene, tamoxifene, droloxifene, lasofoxifene; an agent that results in reduction of A.beta.1-40/1-42, an amyloid aggregation inhibitor, a secretase inhibitor; an osteoporosis agent, droloxifene, fosomax; immunosuppressant agents, FK-506, rapamycin; an anticancer agent, endostatin, angiostatin; a cytotoxic drug, adriamycin, daunomycin, cis-platinum, etoposide, taxol, taxotere; an alkaloid, vincristine; an antimetabolite, methotrexate; a cardiovascular agent, calcium channel blockers; a lipid lowering agent, a statin; a fibrate, a beta-blocker, an ACE inhibitor, an angiotensin-2 receptor antagonist, and a platelet aggregation inhibitor.

25. The method of claim 2, wherein modulation of at least one of cellular membrane potential and cellular membrane conductivity comprises altering at least one of cellular membrane structure or function comprising altering at least one of a conformation, ligand binding activity, and a catalytic activity of a membrane associated protein or constituent.

26. The method of claim 25, wherein the membrane associated protein comprises at least one selected from the group consisting of receptors, transmembrane receptors, ion channel proteins, intracellular attachment proteins, cellular adhesion proteins, integrins, etc.

27. The method of claim 26, wherein the transmembrane receptor comprises a G-Protein Coupled Receptor (GPCR).

28. The method of claim 27, wherein the G-Protein Coupled Receptor (GPCR) interacts with a G protein α subunit.

29. The method of claim 28, wherein the G protein α subunit comprises at least one selected from the group consisting of Gαₛ, Gαᵢ, Gα_{q}, and Gα₁₂.

30. The method of claim 29, wherein the at least one G protein α subunit is Gα_{q}.

31. The method of claim 2, wherein modulation of at least one of cellular membrane potential and cellular membrane conductivity comprises modulating whole-cell conductance.

32. The method of claim 31 wherein modulating whole-cell conductance, comprises modulating at least one of a linear and a non-linear voltage-dependent contribution of the whole-cell conductance.

33. The method of claim 2, wherein modulation of at least one of cellular membrane potential and cellular membrane conductivity comprises modulation of a calcium dependant cellular messaging pathway or system.

34. The method of claim 2, wherein modulation of at least one of cellular membrane potential and cellular membrane conductivity comprises modulation of phospholipase C activity.

35. The method of claim 2, wherein modulation of at least one of cellular membrane potential and cellular membrane conductivity comprises modulation of adenylate cyclase (AC) activity.

36. The method of claim 2, wherein modulation of at least one of cellular membrane potential and cellular membrane conductivity comprises modulation of intracellular signal transduction associated with at least one condition or symptom selected from the group consisting of obstructive airways disease, chronic obstructive pulmonary disease, asthma, rheumatoid arthritis, osteoarthritis, atherosclerosis, cancer, multiple sclerosis, Alzheimer's disease, stroke/cerebral ischemia, head trauma, spinal cord injury, amyotrophic lateral sclerosis, Huntington's disease, Parkinson's disease, migraine, cerebral amyloid angiopathy, AIDS, age-related cognitive decline; mild cognitive impairment and prion diseases.

37. The method of claim 1, comprising administration of the electrokinetic fluid to a cell network or layer, and further comprising modulation of an intercellular junction therein.

38. The method of claim 37, wherein the intracellular junction comprises at least one selected from the group consisting of tight junctions, gap junctions, zona adherins and desmasomes.

39. The method of claim 37, wherein the cell network or layers comprises at least one selected from the group consisting of pulmonary epithelium, bronchial epithelium, and intestinal epithelium.

40. The method of claim 1, wherein the electrokinetically altered aqueous fluid is oxygenated, and wherein the oxygen in the fluid is present in an amount of at least 8 ppm, at least 15, ppm, at least 25 ppm, at least 30 ppm, at least 40 ppm, at least 50 ppm, or at least 60 ppm oxygen at atmospheric pressure.

41. The method of claims 1, wherein the electrokinetically altered aqueous fluid comprises at least one of solvated electrons, and electrokinetically modified or charged oxygen species.

42. The method of claim 41, wherein the form of solvated electrons or electrokinetically modified or charged oxygen species are present in an amount of at least 0.01 ppm, at least 0.1 ppm, at least 0.5 ppm, at least 1 ppm, at least 3 ppm, at least 5 ppm, at least 7 ppm, at least 10 ppm, at least 15 ppm, or at least 20 ppm.

43. The method of claim 42, wherein the electrokinetically altered aqueous fluid comprises a form of solvated electrons stabilized by molecular oxygen.

44. The method of claim 2, wherein the ability to modulate at least one of cellular membrane potential and cellular membrane conductivity persists for at least two, at least three, at least four, at least five, at least 6, at least 12 months, or longer periods, in a closed gas-tight container.

45. The method of claim 1, wherein the amount of oxygen present in charge-stabilized oxygen-containing nanostructures of the electrokinetically-altered fluid is at least 8 ppm, at least 15, ppm, at least 20 ppm, at least 25 ppm, at least 30 ppm, at least 40 ppm, at least 50 ppm, or at least 60 ppm oxygen at atmospheric pressure.

46. The method of claim 1, wherein treating comprises administration by at least one of topical, inhalation, intranasal, and intravenous.
